(19) United States
(12) Patent Application Publication
(10) Pub. No.: US 2026/0015423 A1
(43) Pub. Date: Jan. 15, 2026

(54) ANTIBODIES SPECIFICALLY RECOGNIZING PROGRAMMED CELL DEATH1 LIGAND 1 AND USES THEREOF

(71) Applicant: STAIDSON BIOPHARMA INC., Richmond, CA (US)

(72) Inventors: Leyu LIU, Richmond, CA (US); Wenwu ZHAI, Richmond, CA (US)

(73) Assignee: STAIDSON BIOPHARMA INC., Richmond, CA (US)

(21) Appl. No.: 18/994,762

(22) PCT Filed: Aug. 18, 2023
(86) PCT No.: PCT/US2023/072451

§ 371 Date: Jan. 15, 2025

Related U.S. Application Data
(60) Provisional application No. 63/371,900, filed on Aug. 19, 2022.

(51) Int. Cl. C07K 16/28; A61K 39/00; A61P 37/02

(52) U.S. Cl. CPC C07K 16/2827; A61P 37/02; A61K 2039/505; C07K 2317/24; C07K 2317/33; C07K 2317/565; C07K 2317/622; C07K 2317/626; C07K 2317/76; C07K 2317/92; C07K 2317/94

(57) ABSTRACT

The present application provides antibodies including antigen binding fragment thereof that specifically recognizing programmed cell death 1 ligand 1 (PD-L1). Also provided are methods of making and using these antibodies.

### CROSS-REFERENCE TO RELATED APPLICATION

This application claims priority to U.S. Application 63/371,900, filed 2022 Aug. 19, which are incorporated herein by reference in its entirety.

### REFERENCE TO AN ELECTRONIC SEQUENCE LISTING

The contents of the electronic sequence listing (PDL1_seq list_20230711.xml; Size: 56,955 bytes; and Date of Creation: Jul. 11, 2023) is herein incorporated by reference in its entirety.

### FIELD OF THE APPLICATION

This application pertains to antibodies that specifically recognize programmed cell death 1 ligand 1 (PD-L1), and methods of manufacture and uses thereof, including methods of treating cancer and/or infectious diseases.

### BACKGROUND OF THE APPLICATION

An adaptive immune response involves activation, selection, and clonal proliferation of two major classes of lymphocytes termed T cells and B cells. After encountering an antigen, T cells proliferate and differentiate into antigen specific effector cells, while B cells proliferate and differentiate into antibody-secreting cells. T cell activation is a multistep process requiring several signaling events between the T cell and an antigen-presenting cell (APC). For T cell activation to occur, two types of signals must be delivered to a resting T cell (Baxter and Hodgkin et al., 2002). The first type is mediated by the antigen specific T cell receptor (TCR), and confers specificity to the immune response. The second co-signal type, including co-stimulatory signal and co-inhibitory signal, regulates the magnitude of the response and is delivered through accessory receptors on the T cell. For example, ligation of the CD28 on T cells provided a critical secondary signal along with TCR ligation for native T-cell activation. In contrast, co-inhibitory signaling by the CD28-B7 family is important to regulate immune homeostasis and host defense, as these signals limit the strength and duration of immune responses to prevent autoimmunity. At the same time, microorganisms or tumor cells can use these pathways to establish an immunosuppressive environment to inhibit the immune responses against themselves.

Programmed cell death 1 ligand 1 (PD-L1), also referred to as B7-H1, CD274 (Dong et al. 1999; Freeman et al. 2000), is a 290-amino acid protein type I transmembrane protein that belongs to the immunoglobulin (Ig) superfamily and B7 homologs. PD-L1 contains an Ig-V and Ig-C-like extracellular domain, a transmembrane domain, and a short cytoplasmic tail domain that does not contain canonical signaling motifs (Dong et al. 1999; Keir et al. 2008; Lin et al. 2008). PD-L1, as a ligand, binds to programmed cell death protein 1 (PD-1) receptor which is a member of the CD28 family of receptors (Freeman et al. 2000). Interactions between the extracellular domains of PD-L1 and PD-1 can induce a conformational change in PD-1 that leads to phosphorylation of the cytoplasmic immunoreceptor tyrosine-based inhibitory motif (ITIM) and the immunoreceptor tyrosine-based switch motif (ITSM) by Src family kinases (Gauen et al., 1994; Straus and Weiss, 1992; Zak et al., 2015). These phosphorylated tyrosine motifs subsequently recruit the SHP-2 and SUP-1 protein tyrosine phosphatases to attenuate T cell-activating signals. Engagement of PD-1 by PD-L1 alters the activity of T cells in many ways, inhibiting T cell proliferation, survival, cytokine such as IL-2 production, and other effector functions (Butte et al., 2007; Chang et al., 1999; Curiel et al., 2003; Dong et al., 1999; Freeman et al., 2000; Keir et al., 2006; Latchman et al., 2004). In addition to its interaction with PD-1, PD-L1 can also interact with CD80, which may deliver inhibitory signals to activated T cells and be considered to be important for induction and maintenance of peripheral T cell tolerance (Butte et al., 2007; Park et al., 2010).

PD-L1 is constitutively expressed on immune cells, such as T cells, B cells, macrophages, and dendritic cells (DCs) and also expressed on a variety of nonhematopoietic cells including vascular endothelium, pancreatic islets, and placental syncytiotrophoblasts (Keir et al. 2008). In addition, PD-L1 is expressed by tumor cells as an “adaptive immune mechanism” toescape anti-tumor responses (Ohaegbulam K C et al. 2015) and is upregulated by any inflammatory stimulation (Yamazaki et al. 2002). It has been demonstrated that IFN-γ causes PD-L1 upregulation in ovarian cancer cells, which is responsible for disease progression (Abiko K et al. 2015). PD-L1 acts as a pro-tumorigenic factor in cancer cells via binding to its receptors and activating proliferative and survival signaling pathways (Dong p et al 2017). This finding further indicated that PD-L1 is implicated in subsequent tumor progression.

When PD-L1 expression is induced in the tumor microenvironment in response to proinflammatory cytokines secreted from infiltrating tumor-reactive cytotoxic T lymphocytes (CTLs), binding of PD-L1 to PD-1 on CTL could downregulate anti-tumor immunity. Therefore, blockade of PD-L1/PD-1 pathway is an ideal therapeutic approach to restore and augment anti-tumor immune responses. In pre-clinical models, anti-PD-1 Ab and anti-PD-L1 Ab demonstrated anti-tumor effects in various tumor models, especially those using immunogenic tumors, by enhancing tumor antigen-specific T cell responses including cytokine production, survival, cellular motility, and glycolysis (Chang et al. 2015; Zinselmeyer et al. 2013; Okazaki et al. 2013). In the clinical trials, anti-PD-L1 Ab represented by Atezolizumab, avelumab, and durvalumab have been developed, although they came onto the market later than anti-PD-1 Ab. Atezolizumab was previously developed for the 1st line of breast cancer and ovarian cancer. It is also under development for the treatment of gastro-oesophageal junction cancer as reported in 2021. Avelumab was approved for Merkel cell carcinoma and urothelial carcinoma, and durvalumab was approved for urothelial carcinoma in the USA. WO2006/042237 and WO2010/077634A disclose anti-PD-L1 antibodies and methods to obtain and use them. However, as an optimal therapeutic directed to a target in this pathway has yet to be commercialized, a significant unmet medical need exists

Thus, there remains a need in the art for therapeutic antibodies that effectively inhibit or otherwise antagonize PD-L1, and related methods of treating diseases or conditions mediated through PD-L1, such as cancer or infectious diseases.

The disclosures of all publications, patents, patent applications and published patent applications referred to herein are hereby incorporated herein by reference in their entirety.

### BRIEF SUMMARY OF THE APPLICATION

In some embodiments, there is provided an isolated anti-PD-L1 antibody comprising: a heavy chain variable domain (V_{H}) comprising a heavy chain complementarity determining region (HC-CDR) 1 comprising GFTFGGFG (SEQ ID NO: 1); an HC-CDR2 comprising ITGDSSTI (SEQ ID NO: 6); and an HC-CDR3 comprising VRGPPGTWAY (SEQ ID NO: 11); and a light chain variable domain (V_{L}) comprising a light chain complementarity determining region (LC-CDR) 1 comprising ESVEFYGTTL (SEQ ID NO: 17); an LC-CDR2 comprising GAS (SEQ ID NO: 24); and an LC-CDR3 comprising QQIRKVPWT (SEQ ID NO: 29).

In some embodiments, there is provided an isolated anti-PD-L1 antibody comprising: a V_{H} comprising an HC-CDR1 comprising the amino acid sequence of SEQ ID NO: 1, or a variant thereof comprising up to about 3 amino acid substitutions; an HC-CDR2 comprising the amino acid sequence of SEQ ID NO: 6, or a variant thereof comprising up to about 3 amino acid substitutions; and an HC-CDR3 comprising the amino acid sequence of SEQ ID NO: 11, or a variant thereof comprising up to about 3 amino acid substitutions; and a V_{L} comprising an LC-CDR1 comprising the amino acid sequence of SEQ ID NO: 17, or a variant thereof comprising up to about 3 amino acid substitutions; an LC-CDR2 comprising the amino acid sequence of SEQ ID NO: 24, or a variant thereof comprising up to about 3 amino acid substitutions; and an LC-CDR3 comprising the amino acid sequence of SEQ ID NO: 29, or a variant thereof comprising up to about 3 amino acid substitutions.

In some embodiments, there is provided an isolated anti-PD-L1 antibody comprising a V_{H} comprising an HC-CDR1, an HC-CDR2, and an HC-CDR3 of a V_{H} comprising the amino acid sequence of any one of SEQ ID NOs: 35-39; and a V_{L} comprising an LC-CDR1, an LC-CDR2, and an LC-CDR3 of a V_{L} comprising the amino acid sequence of any one of SEQ ID NOs: 45-48.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 35; and a V_{L} comprising an LC-CDR1, an LC-CDR2, and an LC-CDR3 of a V_{L} comprising the amino acid sequence of SEQ ID NO: 45; (ii) a V_{H} comprising an HC-CDR1, an HC-CDR2, and an HC-CDR3 of a V_{H} comprising the amino acid sequence of SEQ ID NO: 36; and a V_{L} comprising an LC-CDR1, an LC-CDR2, and an LC-CDR3 of a V_{L} comprising the amino acid sequence of SEQ ID NO: 46; (iii) a V_{H} comprising an HC-CDR1, an HC-CDR2, and an HC-CDR3 of a V_{H} comprising the amino acid sequence of SEQ ID NO: 37; and a V_{L} comprising an LC-CDR1, an LC-CDR2, and an LC-CDR3 of a V_{L} comprising the amino acid sequence of SEQ ID NO: 47; (iv) a V_{H} comprising an HC-CDR1, an HC-CDR2, and an HC-CDR3 of a V_{H} comprising the amino acid sequence of SEQ ID NO: 37; and a V_{L} comprising an LC-CDR1, an LC-CDR2, and an LC-CDR3 of a V_{L} comprising the amino acid sequence of SEQ ID NO: 48; (v) a V_{H} comprising an HC-CDR1, an HC-CDR2, and an HC-CDR3 of a V_{H} comprising the amino acid sequence of SEQ ID NO: 37; and a V_{L} comprising an LC-CDR1, an LC-CDR2, and an LC-CDR3 of a V_{L} comprising the amino acid sequence of SEQ ID NO: 46; (vi) a V_{H} comprising an HC-CDR1, an HC-CDR2, and an HC-CDR3 of a V_{H} comprising the amino acid sequence of SEQ ID NO: 38; and a V_{L} comprising an LC-CDR1, an LC-CDR2, and an LC-CDR3 of a V_{L} comprising the amino acid sequence of SEQ ID NO: 47; (vii) a V_{H} comprising an HC-CDR1, an HC-CDR2, and an HC-CDR3 of a V_{H} comprising the amino acid sequence of SEQ ID NO: 38; and a V_{L} comprising an LC-CDR1, an LC-CDR2, and an LC-CDR3 of a V_{L} comprising the amino acid sequence of SEQ ID NO: 48; (viii) a V_{H} comprising an HC-CDR1, an HC-CDR2, and an HC-CDR3 of a V_{H} comprising the amino acid sequence of SEQ ID NO: 38; and a V_{L} comprising an LC-CDR1, an LC-CDR2, and an LC-CDR3 of a V_{L} comprising the amino acid sequence of SEQ ID NO: 46; (ix) a V_{H} comprising an HC-CDR1, an HC-CDR2, and an HC-CDR3 of a V_{H} comprising the amino acid sequence of SEQ ID NO: 39; and a V_{L} comprising an LC-CDR1, an LC-CDR2, and an LC-CDR3 of a V_{L} comprising the amino acid sequence of SEQ ID NO: 47; (x) a V_{H} comprising an HC-CDR1, an HC-CDR2, and an HC-CDR3 of a V_{H} comprising the amino acid sequence of SEQ ID NO: 39; and a V_{L} comprising an LC-CDR1, an LC-CDR2, and an LC-CDR3 of a V_{L} comprising the amino acid sequence of SEQ ID NO: 48; (xi) a V_{H} comprising an HC-CDR1, an HC-CDR2, and an HC-CDR3 of a V_{H} comprising the amino acid sequence of SEQ ID NO: 39; and a V_{L} comprising an LC-CDR1, an LC-CDR2, and an LC-CDR3 of a V_{L} comprising the amino acid sequence of SEQ ID NO: 46; (xii) a V_{H} comprising an HC-CDR1, an HC-CDR2, and an HC-CDR3 of a V_{H} comprising the amino acid sequence of SEQ ID NO: 36; and a V_{L} comprising an LC-CDR1, an LC-CDR2, and an LC-CDR3 of a V_{L} comprising the amino acid sequence of SEQ ID NO: 47; (xiii) a V_{H} comprising an HC-CDR1, an HC-CDR2, and an HC-CDR3 of a V_{H} comprising the amino acid sequence of SEQ ID NO: 36; and a V_{L} comprising an LC-CDR1, an LC-CDR2, and an LC-CDR3 of a V_{L} comprising the amino acid sequence of SEQ ID NO: 48.

In some embodiments, there is provided an isolated anti-PD-L1 antibody comprising: (i) a V_{H} comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11, or a variant thereof comprising up to about 5 amino acid substitutions in the HC-CDRs; and a V_{L} comprising an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of any one of SEQ ID NOs: 35-39, or a variant thereof having at least about 80% sequence identity to the amino acid sequence of any one of SEQ ID NOs: 35-39; and a V_{L} comprising the amino acid sequence of any one of SEQ ID NOs: 45-48, or a variant thereof having at least about 80% sequence identity to the amino acid sequence of any one of SEQ ID NOs: 45-48.

In some embodiments, the isolated anti-PD-L1 antibody comprises: (i) a V_{H} comprising the amino acid sequence of SEQ ID NO: 35, or a variant thereof having at least about 80% sequence identity to the amino acid sequence of SEQ ID NO: 35; and a V_{L} comprising the amino acid sequence of SEQ ID NO: 45, or a variant thereof having at least about 80% sequence identity to the amino acid sequence of SEQ ID NO: 45; (ii) a V_{H} comprising the amino acid sequence of SEQ ID NO: 36, or a variant thereof having at least about 80% sequence identity to the amino acid sequence of SEQ ID NO: 36; and a V_{L} comprising the amino acid sequence of SEQ ID NO: 46, or a variant thereof having at least about 80% sequence identity to the amino acid sequence of SEQ ID NO: 46; (iii) a V_{H} comprising the amino acid sequence of SEQ ID NO: 37, or a variant thereof having at least about 80% sequence identity to the amino acid sequence of SEQ ID NO: 37; and a V_{L} comprising the amino acid sequence of SEQ ID NO: 47, or a variant thereof having at least about 80% sequence identity to the amino acid sequence of SEQ ID NO: 47; (iv) a V_{H} comprising the amino acid sequence of SEQ ID NO: 37, or a variant thereof having at least about 80% sequence identity to the amino acid sequence of SEQ ID NO: 37; and a V_{L} comprising the amino acid sequence of SEQ ID NO: 48, or a variant thereof having at least about 80% sequence identity to the amino acid sequence of SEQ ID NO: 48; (v) a V_{H} comprising the amino acid sequence of SEQ ID NO: 37, or a variant thereof having at least about 80% sequence identity to the amino acid sequence of SEQ ID NO: 37; and a V_{L} comprising the amino acid sequence of SEQ ID NO: 46, or a variant thereof having at least about 80% sequence identity to the amino acid sequence of SEQ ID NO: 46; (vi) a V_{H} comprising the amino acid sequence of SEQ ID NO: 38, or a variant thereof having at least about 80% sequence identity to the amino acid sequence of SEQ ID NO: 38; and a V_{L} comprising the amino acid sequence of SEQ ID NO: 47, or a variant thereof having at least about 80% sequence identity to the amino acid sequence of SEQ ID NO: 47; (vii) a V_{H} comprising the amino acid sequence of SEQ ID NO: 38, or a variant thereof having at least about 80% sequence identity to the amino acid sequence of SEQ ID NO: 38; and a V_{L} comprising the amino acid sequence of SEQ ID NO: 48, or a variant thereof having at least about 80% sequence identity to the amino acid sequence of SEQ ID NO: 48; (viii) a V_{H} comprising the amino acid sequence of SEQ ID NO: 38, or a variant thereof having at least about 80% sequence identity to the amino acid sequence of SEQ ID NO: 38; and a V_{L} comprising the amino acid sequence of SEQ ID NO: 46, or a variant thereof having at least about 80% sequence identity to the amino acid sequence of SEQ ID NO: 46; (ix) a V_{H} comprising the amino acid sequence of SEQ ID NO: 39, or a variant thereof having at least about 80% sequence identity to the amino acid sequence of SEQ ID NO: 39; and a V_{L} comprising the amino acid sequence of SEQ ID NO: 47, or a variant thereof having at least about 80% sequence identity to the amino acid sequence of SEQ ID NO: 47; (x) a V_{H} comprising the amino acid sequence of SEQ ID NO: 39, or a variant thereof having at least about 80% sequence identity to the amino acid sequence of SEQ ID NO: 39; and a V_{L} comprising the amino acid sequence of SEQ ID NO: 48, or a variant thereof having at least about 80% sequence identity to the amino acid sequence of SEQ ID NO: 48; (xi) a V_{H} comprising the amino acid sequence of SEQ ID NO: 39, or a variant thereof having at least about 80% sequence identity to the amino acid sequence of SEQ ID NO: 39; and a V_{L} comprising the amino acid sequence of SEQ ID NO: 46, or a variant thereof having at least about 80% sequence identity to the amino acid sequence of SEQ ID NO: 46; (xii) a V_{H} comprising the amino acid sequence of SEQ ID NO: 36, or a variant thereof having at least about 80% sequence identity to the amino acid sequence of SEQ ID NO: 36; and a V_{L} comprising the amino acid sequence of SEQ ID NO: 47, or a variant thereof having at least about 80% sequence identity to the amino acid sequence of SEQ ID NO: 47; (xiii) a V_{H} comprising the amino acid sequence of SEQ ID NO: 36, or a variant thereof having at least about 80% sequence identity to the amino acid sequence of SEQ ID NO: 36; and a V_{L} comprising the amino acid sequence of SEQ ID NO: 48, or a variant thereof having at least about 80% sequence identity to the amino acid sequence of SEQ ID NO: 48.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 40; and a V_{L} comprising an LC-CDR1, an LC-CDR2, and an LC-CDR3 of a V_{L} comprising the amino acid sequence of SEQ ID NO: 49.

In some embodiments, there is provided an isolated anti-PD-L1 antibody comprising: (i) a V_{H} comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 12, or a variant thereof comprising up to about 5 amino acid substitutions in the HC-CDRs; and a V_{L} comprising an LC-CDR1 comprising the amino acid sequence of SEQ ID NO: 18, an LC-CDR2 comprising the amino acid sequence of SEQ ID NO: 25, and an LC-CDR3 comprising the amino acid sequence of SEQ ID NO: 30, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of SEQ ID NO: 40, or a variant thereof having at least about 80% sequence identity to the amino acid sequence of SEQ ID NO: 40; and a V_{L} comprising the amino acid sequence of SEQ ID NO: 49, or a variant thereof having at least about 80% sequence identity to the amino acid sequence of SEQ ID NO: 49.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 41; and a V_{L} comprising an LC-CDR1, an LC-CDR2, and an LC-CDR3 of a V_{L} comprising the amino acid sequence of SEQ ID NO: 50.

In some embodiments, there is provided an isolated anti-PD-L1 antibody comprising: (i) a V_{H} comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 13, or a variant thereof comprising up to about 5 amino acid substitutions in the HC-CDRs; and a V_{L} comprising an LC-CDR1 comprising the amino acid sequence of SEQ ID NO: 19, an LC-CDR2 comprising the amino acid sequence of SEQ ID NO: 25, and an LC-CDR3 comprising the amino acid sequence of SEQ ID NO: 30, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of SEQ ID NO: 41, or a variant thereof having at least about 80% sequence identity to the amino acid sequence of SEQ ID NO: 41; and a V_{L} comprising the amino acid sequence of SEQ ID NO: 50, or a variant thereof having at least about 80% sequence identity to the amino acid sequence of SEQ ID NO: 50.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 42; and a V_{L} comprising an LC-CDR1, an LC-CDR2, and an LC-CDR3 of a V_{L} comprising the amino acid sequence of SEQ ID NO: 51.

In some embodiments, there is provided an isolated anti-PD-L1 antibody comprising: (i) a V_{H} comprising an HC-CDR1 comprising the amino acid sequence of SEQ ID NO: 3, an HC-CDR2 comprising the amino acid sequence of SEQ ID NO: 8, and an HC-CDR3 comprising the amino acid sequence of SEQ ID NO: 14, or a variant thereof comprising up to about 5 amino acid substitutions in the HC-CDRs; and a V_{L} comprising an LC-CDR1 comprising the amino acid sequence of SEQ ID NO: 20, an LC-CDR2 comprising the amino acid sequence of SEQ ID NO: 26, and an LC-CDR3 comprising the amino acid sequence of SEQ ID NO: 31, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of SEQ ID NO: 42, or a variant thereof having at least about 80% sequence identity to the amino acid sequence of SEQ ID NO: 42; and a V_{L} comprising the amino acid sequence of SEQ ID NO: 51, or a variant thereof having at least about 80% sequence identity to the amino acid sequence of SEQ ID NO: 51.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 43; and a V_{L} comprising an LC-CDR1, an LC-CDR2, and an LC-CDR3 of a V_{L} comprising the amino acid sequence of SEQ ID NO: 52.

In some embodiments, there is provided an isolated anti-PD-L1 antibody comprising: (i) a V_{H} comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15, or a variant thereof comprising up to about 5 amino acid substitutions in the HC-CDRs; and a V_{L} comprising an LC-CDR1 comprising the amino acid sequence of SEQ ID NO: 21, an LC-CDR2 comprising the amino acid sequence of SEQ ID NO: 26, and an LC-CDR3 comprising the amino acid sequence of SEQ ID NO: 32, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of SEQ ID NO: 43, or a variant thereof having at least about 80% sequence identity to the amino acid sequence of SEQ ID NO: 43; and a V_{L} comprising the amino acid sequence of SEQ ID NO: 52, or a variant thereof having at least about 80% sequence identity to the amino acid sequence of SEQ ID NO: 52.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 43; and a V_{L} comprising an LC-CDR1, an LC-CDR2, and an LC-CDR3 of a V_{L} comprising the amino acid sequence of SEQ ID NO: 53.

In some embodiments, there is provided an isolated anti-PD-L1 antibody comprising: (i) a V_{H} comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15, or a variant thereof comprising up to about 5 amino acid substitutions in the HC-CDRs; and a V_{L} comprising an LC-CDR1 comprising the amino acid sequence of SEQ ID NO: 22, an LC-CDR2 comprising the amino acid sequence of SEQ ID NO: 27, and an LC-CDR3 comprising the amino acid sequence of SEQ ID NO: 33, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of SEQ ID NO: 43, or a variant thereof having at least about 80% sequence identity to the amino acid sequence of SEQ ID NO: 43; and a V_{L} comprising the amino acid sequence of SEQ ID NO: 53, or a variant thereof having at least about 80% sequence identity to the amino acid sequence of SEQ ID NO: 53.

In some embodiments, there is provided an isolated anti-PD-L1 antibody comprising: (i) a V_{H} comprising an HC-CDR1, an HC-CDR2, and an HC-CDR3 of a V_{H} comprising the amino acid sequence of SEQ ID NO: 44; and a V_{L} comprising an LC-CDR1, an LC-CDR2, and an LC-CDR3 of a V_{L} comprising the amino acid sequence of SEQ ID NO: 54.

In some embodiments, there is provided an isolated anti-PD-L1 antibody comprising: (i) a V_{H} comprising an HC-CDR1 comprising the amino acid sequence of SEQ ID NO: 5, an HC-CDR2 comprising the amino acid sequence of SEQ ID NO: 10, and an HC-CDR3 comprising the amino acid sequence of SEQ ID NO: 16, or a variant thereof comprising up to about 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 28, and an LC-CDR3 comprising the amino acid sequence of SEQ ID NO: 34, or a variant thereof comprising up to about 5 amino acid substitutions in the LC-CDRs.

In some embodiments, according to any one of the isolated anti-PD-L1 antibodies described above, the isolated anti-PD-L1 antibody comprises: a V_{H} comprising the amino acid sequence of SEQ ID NO: 44, or a variant thereof having at least about 80% sequence identity to the amino acid sequence of SEQ ID NO: 44; and a V_{L} comprising the amino acid sequence of SEQ ID NO: 54, or a variant thereof having at least about 80% sequence identity to the amino acid sequence of SEQ ID NO: 54.

In some embodiments, there is provided an isolated anti-PD-L1 antibody that specifically binds to the human PD-L1 with a Kd from about 0.1 pM to 1 nM.

In some embodiments, there is provided an isolated anti-PD-L1 antibody that specifically binds to PD-L1 competitively with any one of the isolated anti-PD-L1 antibodies described above. In some embodiments, there is provided an isolated anti-PD-L1 antibody that specifically binds to the same epitope as any one of isolated anti-PD-L1 antibodies described above.

In some embodiments according to any of the isolated anti-PD-L1 antibodies described above, the isolated anti-PD-L1 antibody comprises an Fc fragment. In some embodiments, the isolated anti-PD-L1 antibody is a full-length IgG antibody. In some embodiments, the isolated anti-PD-L1 antibody is a full-length IgG1, IgG2, IgG3, or IgG4 antibody. In some embodiments, the anti-PD-L1 antibody is a chimeric, human, or humanized antibody. In some embodiments, the anti-PD-L1 antibody is an antigen binding fragment selected from the group consisting of a Fab, a Fab′, a F(ab)′2, a Fab′-SH, a single-chain Fv (scFv), an Fv fragment, a dAb, a Fd, a nanobody, a diabody, and a linear antibody.

In some embodiments, there is provided isolated nucleic acid molecule(s) that encodes any one of the anti-PD-L1 antibodies described above. In some embodiments, there is provided a vector comprising any one of the nucleic acid molecules described above. In some embodiments, there is provided a host cell comprising any one of the anti-PD-L1 antibodies described above, any one of the nucleic acid molecules described above, or any one of the vectors described above. In some embodiments, there is provided a method of producing an anti-PD-L1 antibody, comprising: a) culturing any one of the host cells described above under conditions effective to express the anti-PD-L1 antibody; and b) obtaining the expressed anti-PD-L1 antibody from the host cell.

In some embodiments, there is provided a method of treating a disease or condition in an individual in need thereof, comprising administering to the individual an effective amount of any one of the anti-PD-L1 antibodies described above. In some embodiments, there is provided the use of any one of the anti-PD-L1 antibodies described herein for the preparation of pharmaceutical compositions for treating a disease or condition in an individual in need. In some embodiments, provided is the use of any one of the anti-PD-L1 antibodies described above, or a pharmaceutical composition comprising any one of anti-PD-L1 antibodies described above in the manufacture of a medicament for treating a disease or condition. In some embodiments, the disease or condition is associated with PD-L1, comprising cancer or infectious disease or condition. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, esophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus.

Also provided are pharmaceutical compositions, kits and articles of manufacture comprising any one of the anti-PD-L1 antibodies described above.

### BRIEF DESCRIPTION OF THE DRAWINGS

FIGS. 1A-1C show the results of ligand blocking assays of exemplary chimeric anti-human PD-L1 antibodies that block PD-L1 binding to PD1 as analyzed by HTRF. FIG. 1A shows the result of ligand blocking assay of exemplary chimeric anti-human PD-L1 antibodies Mab1-01 and Mab1-02 that block PD-L1 binding to PD1 as analyzed by HTRF. FIG. 1B shows the result of ligand blocking assay of exemplary chimeric anti-human PD-L1 antibody Mab2-04 that blocks PD-L1 binding to PD1 as analyzed by HTRF. FIG. 1C shows the result of ligand blocking assay of exemplary chimeric anti-human PD-L1 antibodies Mab3-14, Mab3-16, Mab3-17 and Mab3-18 that block PD-L1 binding to PD1 as analyzed by HTRF.

FIGS. 2A-2E show the enhanced production of IL-2 by exemplary chimeric anti-human PD-L1 antibodies in Jurkat-Raji reporter cell assay. FIG. 2A shows the enhanced production of IL-2 by exemplary chimeric anti-human PD-L1 antibodies Mab1-01 and Mab1-02 in Jurkat-Raji reporter cell assay. FIG. 2B shows the enhanced production of IL-2 by exemplary chimeric anti-human PD-L1 antibody Mab2-04 in Jurkat-Raji reporter cell assay. FIG. 2C shows the enhanced production of IL-2 by exemplary chimeric anti-human PD-L1 antibody Mab3-14 in Jurkat-Raji reporter cell assay. FIG. 2D shows the enhanced production of IL-2 by exemplary chimeric anti-human PD-L1 antibodies Mab3-16 and Mab3-17 in Jurkat-Raji reporter cell assay. FIG. 2E shows the enhanced production of IL-2 by exemplary chimeric anti-human PD-L1 antibody Mab3-18 in Jurkat-Raji reporter cell assay.

FIG. 3 shows that the enhanced T cell activities represented as IL-2 production level by exemplary chimeric anti-human PD-L1 antibody Mab 3-16 in SEB stimulated PBMCs.

FIG. 4 shows that the IFN-γ secretion promoted by exemplary chimeric anti-human PD-L1 antibody Mab 3-16 in a mixed lymphocyte reaction (MLR).

FIG. 5A-5B show the results of ligand blocking assay of exemplary humanized anti-human PD-L1 antibodies that block PD-L1 binding to PD1 as analyzed by HTRF.

FIG. 6A shows the enhanced production of IL-2 by exemplary humanized anti-human PD-L1 antibody Hum-7 in Jurkat-Raji reporter cell assay. FIG. 6B shows the enhanced production of IL-2 by exemplary humanized anti-human PD-L1 antibodies Hum-5 and Hum-6 in Jurkat-Raji reporter cell assay.

FIG. 7A shows the enhanced T cell activity represented as IL-2 production level by exemplary humanized anti-human PD-L1 antibody Hum-6 in SEB stimulated PBMCs. FIG. 7B shows the enhanced T cell activity represented as IL-2 production level by exemplary humanized anti-human PD-L1 antibodies Hum-5 and Hum-7 in SEB stimulated PBMCs.

FIG. 8 shows the results of Pharmacokinetics (PK) study of exemplary humanized anti-human PD-L1 antibody Hum-6 in rats.

### DETAILED DESCRIPTION OF THE APPLICATION

The present application in one aspect provides an isolated anti-PD-L1 antibody that specifically binds to human and/or cynomolgus monkey PD-L1. By using a combination of hybridoma technology, humanization of antibody and appropriately designed biochemical and biological assays, we have identified highly potent antibody molecules that bind to human and/or cynomolgus monkey PD-L1 and inhibit the action of human and/or cynomolgus monkey PD-L1 to its receptor. The results presented herein indicate that the present application antibodies bind human and/or cynomolgus monkey PD-L1 with high affinity and biological activity.

The anti-PD-L1 antibodies provided by the present application include, for example, full-length anti-PD-L1 antibodies, anti-PD-L1 scFvs, anti-PD-L1 Fc fusion proteins, multi-specific (such as bispecific) anti-PD-L1 antibodies, anti-PD-L1 immunoconjugates, and the like.

In some embodiments, the isolated anti-PD-L1 antibody comprises: a heavy chain variable domain (V_{H}) comprising a heavy chain complementarity determining region (HC-CDR) 1 comprising GFTFGGFG (SEQ ID NO: 1); an HC-CDR2 comprising ITGDSSTI (SEQ ID NO: 6); and an HC-CDR3 comprising VRGPPGTWAY (SEQ ID NO: 11); and a light chain variable domain (V_{L}) comprising a light chain complementarity determining region (LC-CDR) 1 comprising ESVEFYGTTL (SEQ ID NO: 17); an LC-CDR2 comprising GAS (SEQ ID NO: 24); and an LC-CDR3 comprising QQIRKVPWT (SEQ ID NO: 29).

In some embodiments, the isolated anti-PD-L1 antibody comprises: a heavy chain variable domain (V_{H}) comprising a heavy chain complementarity determining region (HC-CDR) 1 comprising GYSFSGYF (SEQ ID NO: 2); an HC-CDR2 comprising INPSNDDT (SEQ ID NO: 7); and an HC-CDR3 comprising ARFGHASWYFDV (SEQ ID NO: 12); and a light chain variable domain (V_{L}) comprising a light chain complementarity determining region (LC-CDR) 1 comprising SSVIY (SEQ ID NO: 18); an LC-CDR2 comprising DTS (SEQ ID NO: 25); and an LC-CDR3 comprising QQWSTYPLT (SEQ ID NO: 30).

In some embodiments, the isolated anti-PD-L1 antibody comprises: a heavy chain variable domain (V_{H}) comprising a heavy chain complementarity determining region (HC-CDR) 1 comprising GYSFSGYF (SEQ ID NO: 2); an HC-CDR2 comprising INPSNDDT (SEQ ID NO: 7); and an HC-CDR3 comprising ARFGHASWYLDV (SEQ ID NO: 13); and a light chain variable domain (V_{L}) comprising a light chain complementarity determining region (LC-CDR) 1 comprising SSIIY (SEQ ID NO: 19); an LC-CDR2 comprising DTS (SEQ ID NO: 25); and an LC-CDR3 comprising QQWSTYPLT (SEQ ID NO: 30).

In some embodiments, the isolated anti-PD-L1 antibody comprises: a heavy chain variable domain (V_{H}) comprising a heavy chain complementarity determining region (HC-CDR) 1 comprising GYTFTDYA (SEQ ID NO: 3); an HC-CDR2 comprising IIPSGDHT (SEQ ID NO: 8); and an HC-CDR3 comprising ARGHGFHLYVMDY (SEQ ID NO: 14); and a light chain variable domain (V_{L}) comprising a light chain complementarity determining region (LC-CDR) 1 comprising QSIADY (SEQ ID NO: 20); an LC-CDR2 comprising YAS (SEQ ID NO: 26); and an LC-CDR3 comprising QNGHSFPPT (SEQ ID NO: 31).

In some embodiments, the isolated anti-PD-L1 antibody comprises: a heavy chain variable domain (V_{H}) comprising a heavy chain complementarity determining region (HC-CDR) 1 comprising GDSITSGY (SEQ ID NO: 4); an HC-CDR2 comprising VSYSGST (SEQ ID NO: 9); and an HC-CDR3 comprising ANYYGDNWPFPLDY (SEQ ID NO: 15); and a light chain variable domain (V_{L}) comprising a light chain complementarity determining region (LC-CDR) 1 comprising QSINGN (SEQ ID NO: 21); an LC-CDR2 comprising YAS (SEQ ID NO: 26); and an LC-CDR3 comprising QNGHGFPYT (SEQ ID NO: 32).

In some embodiments, the isolated anti-PD-L1 antibody comprises: a heavy chain variable domain (V_{H}) comprising a heavy chain complementarity determining region (HC-CDR) 1 comprising GDSITSGY (SEQ ID NO: 4); an HC-CDR2 comprising VSYSGST (SEQ ID NO: 9); and an HC-CDR3 comprising ANYYGDNWPFPLDY (SEQ ID NO: 15); and a light chain variable domain (V_{L}) comprising a light chain complementarity determining region (LC-CDR) 1 comprising KSLLHSNGNTY (SEQ ID NO: 22); an LC-CDR2 comprising RMS (SEQ ID NO: 27); and an LC-CDR3 comprising MQHLEYPFT (SEQ ID NO: 33).

In some embodiments, the isolated anti-PD-L1 antibody comprises: a heavy chain variable domain (V_{H}) comprising a heavy chain complementarity determining region (HC-CDR) 1 comprising GYTFSMYY (SEQ ID NO: 5); an HC-CDR2 comprising INPSNGRT (SEQ ID NO: 10); and an HC-CDR3 comprising TREISYDEFAY (SEQ ID NO: 16); and a light chain variable domain (V_{L}) comprising a light chain complementarity determining region (LC-CDR) 1 comprising QSLVHSNGNTY (SEQ ID NO: 23); an LC-CDR2 comprising RVS (SEQ ID NO: 28); and an LC-CDR3 comprising SQSAHLPYT (SEQ ID NO: 34).

Also provided are nucleic acids encoding the anti-PD-L1 antibodies, compositions comprising the anti-PD-L1 antibodies, and methods of making and using the anti-PD-L1 antibodies.

### Definitions

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more of other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume for cancer). The methods of the application contemplate any one or more of these aspects of treatment.

The term “antibody” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein includes an antibody fragment including, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody, a nanobody, a domain antibody, a bivalent domain antibody, or any other antibody fragments that bind to an antigen but do not comprise a complete antibody structure. An antigen-binding fragment also includes a fusion protein comprising the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody “competes” for binding to a target PD-L1 with a second antibody when the first antibody inhibits target PD-L1 binding of the second antibody by at least about 50% (such as at least about any of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731.

As used herein, the term “specifically binds”, “specifically recognizing”, or “is specific for” refers to measurable and reproducible interactions, such as binding between a target and an antibody that is determinative of the presence of the target in the presence of a heterogeneous population of molecules, including biological molecules. For example, an antibody that specifically recognizes a target (which can be an epitope) is an antibody that binds to this target with greater affinity, avidity, more readily, and/or with greater duration than its binding to other targets. In some embodiments, an antibody that specifically recognizes an antigen reacts with one or more antigenic determinants of the antigen with a binding affinity that is at least about 10 times its binding affinity for other targets.

An “isolated” anti-PD-L1 antibody as used herein refers to an anti-PD-L1 antibody that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “chimeric antibody” refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this application (see U.S. Pat. No. 4,816,567; and Morrison et al., Proc. Natl. Acad. Sci. USA, 81:6851-6855 (1984)).

“Fv” is the minimum antibody fragment which contains a complete antigen-recognition and binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv”, also abbreviated as “sFv” or “scFv”, are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skilled in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of this application is one that binds to an IgG antibody (a 7 receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daeron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” comprises the stretch of residues of C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC₅₀ value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent Kd or IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5-fold to about 100-fold, e.g. from about 25 to about 50-fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-PD-L1 antibody or composition as disclosed herein, is an amount sufficient to carry out a specifically stated purpose. An “effective amount” can be determined empirically and by known methods relating to the stated purpose.

The term “therapeutically effective amount” refers to an amount of an anti-PD-L1 antibody or composition as disclosed herein, effective to “treat” a disease or disorder in an individual. In the case of cancer, the therapeutically effective amount of the anti-PD-L1 antibody or composition as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the anti-PD-L1 antibody or composition as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that extends the survival of a patient. In some embodiments, the therapeutically effective amount is an amount that improves progression free survival of a patient.

As used herein, by “pharmaceutically acceptable” or “pharmacologically compatible” is meant a material that is not biological or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a”, “an”, and “the” include plural referents unless the context clearly dictates otherwise.

### Anti-PD-L1 Antibodies

In one aspect, the present application provides anti-PD-L1 antibodies that specifically bind to human and/or cynomolgus monkey PD-L1. Anti-PD-L1 antibodies include, but are not limited to, humanized antibodies, chimeric antibodies, mouse antibodies, human antibodies, and antibodies comprising the heavy chain and/or light chain CDRs discussed herein. In one aspect, the present application provides isolated antibodies that bind to PD-L1. Contemplated anti-PD-L1 antibodies include, for example, full-length anti-PD-L1 antibodies (e.g., full-length IgG1 or IgG4), anti-PD-L1 scFvs, anti-PD-L1 Fc fusion proteins, multi-specific (such as bispecific) anti-PD-L1 antibodies, anti-PD-L1 immunoconjugates, and the like. In some embodiments, the anti-PD-L1 antibody is a full-length antibody (e.g., full-length IgG1 or IgG4) or antigen-binding fragment thereof, which specifically binds to PD-L1. In some embodiments, the anti-PD-L1 antibody is a Fab, a Fab′, a F(ab)′₂, a Fab′-SH, a single-chain Fv (scFv), an Fv fragment, a dAb, a Fd, a nanobody, a diabody, or a linear antibody. In some embodiments, reference to an antibody that specifically binds to PD-L1 means that the antibody binds to PD-L1 with an affinity that is at least about 10 times (including for example at least about any one of 10, 10², 10³, 10⁴, 10⁵, 10⁶, or 10⁷ times) more tightly than its binding affinity for a non-target. In some embodiments, the non-target is an antigen that is not PD-L1. Binding affinity can be determined by methods known in the art, such as ELISA, fluorescence activated cell sorting (FACS) analysis, or radioimmunoprecipitation assay (RIA). Kd can be determined by methods known in the art, such as surface plasmon resonance (SPR) assay or biolayer interferometry (BLI).

Although anti-PD-L1 antibodies containing human sequences (e.g., human heavy and light chain variable domain sequences comprising human CDR sequences) are extensively discussed herein, non-human anti-PD-L1 antibodies are also contemplated. In some embodiments, non-human anti-PD-L1 antibodies comprise human CDR sequences from an anti-PD-L1 antibody as described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. In some embodiments, a non-human anti-PD-L1 antibody includes an anti-PD-L1 antibody generated by grafting one or more human CDR sequences as described herein onto a non-human framework sequence (e.g., a mouse or chicken framework sequence).

The amino acid sequence of an exemplary extracellular domain (ECD) of human PD-L1 comprises or consists of the amino acid sequence of SEQ ID NO: 59. The amino acid sequence of an exemplary ECD of mouse PD-L1 comprises or consists of the amino acid sequence of SEQ ID NO:60.

In some embodiments, the anti-PD-L1 antibody described herein specifically recognizes an epitope within human PD-L1. In some embodiments, the anti-PD-L1 antibody cross-reacts with PD-L1 from species other than human. In some embodiments, the anti-PD-L1 antibody is completely specific for human PD-L1 and does not exhibit cross-reactivity with PD-L1 from other non-human species.

In some embodiments, the anti-PD-L1 antibody cross-reacts with at least one allelic variant of the PD-L1 protein (or fragments thereof). In some embodiments, the allelic variant has up to about 30 (such as about any of 1, 2, 3, 4, 5, 6, 7, 8, 9, 10, 15, 20, 25, or 30) amino acid substitutions (such as a conservative substitution) when compared to the naturally occurring PD-L1 (or fragments thereof). In some embodiments, the anti-PD-L1 antibody does not cross-react with any allelic variants of the PD-L1 protein (or fragments thereof).

In some embodiments, the anti-PD-L1 antibody cross-reacts with at least one interspecies variant of the PD-L1 protein. In some embodiments, for example, the PD-L1 protein (or fragments thereof) is human PD-L1 and the interspecies variant of the PD-L1 protein (or fragments thereof) is a cynomolgus monkey variant thereof. In some embodiments, the anti-PD-L1 antibody does not cross-react with any interspecies variants of the PD-L1 protein.

In some embodiments, according to any of the anti-PD-L1 antibodies described herein, the anti-PD-L1 antibody comprises an antibody heavy chain constant region and an antibody light chain constant region. In some embodiments, the anti-PD-L1 antibody comprises an IgG1 heavy chain constant region. In some embodiments, the anti-PD-L1 antibody comprises an IgG2 heavy chain constant region. In some embodiments, the anti-PD-L1 antibody comprises an IgG3 heavy chain constant region. In some embodiments, the anti-PD-L1 antibody comprises an IgG4 heavy chain constant region. In some embodiments, the heavy chain constant region comprises (including consisting of or consisting essentially of) the amino acid sequence of SEQ ID NO: 55. In some embodiments, the heavy chain constant region comprises (including consisting of or consisting essentially of) the amino acid sequence of SEQ ID NO: 56. In some embodiments, the anti-PD-L1 antibody comprises a kappa light chain constant region. In some embodiments, the light chain constant region comprises (including consisting of or consisting essentially of) the amino acid sequence of SEQ ID NO: 57. In some embodiments, the anti-PD-L1 antibody comprises a lambda light chain constant region. In some embodiments, the light chain constant region comprises (including consisting of or consisting essentially of) the amino acid sequence of SEQ ID NO: 58. In some embodiments, the anti-PD-L1 antibody comprises an antibody heavy chain variable domain and an antibody light chain variable domain.

In some embodiments, the isolated anti-PD-L1 antibody comprises: a heavy chain variable domain (V_{H}) comprising a heavy chain complementarity determining region (HC-CDR) 1 comprising GFTFGGFG (SEQ ID NO: 1); an HC-CDR2 comprising ITGDSSTI (SEQ ID NO: 6); and an HC-CDR3 comprising VRGPPGTWAY (SEQ ID NO: 11); and a light chain variable domain (V_{L}) comprising a light chain complementarity determining region (LC-CDR) 1 comprising ESVEFYGTTL (SEQ ID NO: 17); an LC-CDR2 comprising GAS (SEQ ID NO: 24); and an LC-CDR3 comprising QQIRKVPWT (SEQ ID NO: 29)

In some embodiments, the anti-PD-L1 antibody comprises a V_{H} comprising: an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of SEQ ID NO: 6, or a variant thereof comprising up to about 3 (such as about any of 1, 2, or 3) amino acid substitutions, and an HC-CDR3 comprising the amino acid sequence of SEQ ID NO: 11, or a variant thereof comprising up to about 3 (such as about any of 1, 2, or 3) amino acid substitutions.

In some embodiments, the anti-PD-L1 antibody comprises a V_{H} comprising: an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11.

In some embodiments, the anti-PD-L1 antibody comprises a V_{L} comprising: an LC-CDR1 comprising the amino acid sequence of SEQ ID NO: 17, or a variant thereof comprising up to about 3 (such as about any of 1, 2, or 3) amino acid substitutions, an LC-CDR2 comprising the amino acid sequence of SEQ ID NO: 24, or a variant thereof comprising up to about 3 (such as about any of 1, 2, or 3) amino acid substitutions, and an LC-CDR3 comprising the amino acid sequence of SEQ ID NO: 29, or a variant thereof comprising up to about 3 (such as about any of 1, 2, or 3) amino acid substitutions.

In some embodiments, the anti-PD-L1 antibody comprises a V_{L} comprising: an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29.

In some embodiments, the anti-PD-L1 antibody comprises a V_{H} comprising: an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of SEQ ID NO: 6, or a variant thereof comprising up to about 3 (such as about any of 1, 2, or 3) amino acid substitutions, and an HC-CDR3 comprising the amino acid sequence of SEQ ID NO: 11, or a variant thereof comprising up to about 3 (such as about any of 1, 2, or 3) amino acid substitutions; and a V_{L} comprising: an LC-CDR1 comprising the amino acid sequence of SEQ ID NO: 17, or a variant thereof comprising up to about 3 (such as about any of 1, 2, or 3) amino acid substitutions, an LC-CDR2 comprising the amino acid sequence of SEQ ID NO: 24, or a variant thereof comprising up to about 3 (such as about any of 1, 2, or 3) amino acid substitutions, and an LC-CDR3 comprising the amino acid sequence of SEQ ID NO: 29, or a variant thereof comprising up to about 3 (such as about any of 1, 2, or 3) amino acid substitutions.

In some embodiments, the anti-PD-L1 antibody comprises a V_{H} comprising: an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11; and a V_{L} comprising: an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29.

In some embodiments, the anti-PD-L1 antibody comprises a V_{H} comprising: an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11, or a variant thereof comprising up to about 5 amino acid substitutions in the HC-CDRs; and a V_{L} comprising an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11; and a V_{L} comprising an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29.

In some embodiments, the anti-PD-L1 antibody comprises a V_{H} comprising an HC-CDR1, an HC-CDR2 and an HC-CDR3 of the V_{H} comprising the amino acid sequence of any one of SEQ ID NOs: 35-39; and a V_{L} comprising an LC-CDR1, an LC-CDR2, and an LC-CDR3 of the V_{L} comprising the amino acid sequence of any one of SEQ ID NOs: 45-48.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 35, and a V_{L} comprising an LC-CDR1, an LC-CDR2 and an LC-CDR3 of the V_{L} comprising the amino acid sequence of SEQ ID NO: 45.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 36, and a V_{L} comprising an LC-CDR1, an LC-CDR2 and an LC-CDR3 of the V_{L} comprising the amino acid sequence of SEQ ID NO: 46.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 37, and a V_{L} comprising an LC-CDR1, an LC-CDR2 and an LC-CDR3 of the V_{L} comprising the amino acid sequence of SEQ ID NO: 47.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 37, and a V_{L} comprising an LC-CDR1, an LC-CDR2 and an LC-CDR3 of the V_{L} comprising the amino acid sequence of SEQ ID NO: 48.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 37, and a V_{L} comprising an LC-CDR1, an LC-CDR2 and an LC-CDR3 of the V_{L} comprising the amino acid sequence of SEQ ID NO: 46.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 38, and a V_{L} comprising an LC-CDR1, an LC-CDR2 and an LC-CDR3 of the V_{L} comprising the amino acid sequence of SEQ ID NO: 47.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 38, and a V_{L} comprising an LC-CDR1, an LC-CDR2 and an LC-CDR3 of the V_{L} comprising the amino acid sequence of SEQ ID NO: 48.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 38, and a V_{L} comprising an LC-CDR1, an LC-CDR2 and an LC-CDR3 of the V_{L} comprising the amino acid sequence of SEQ ID NO: 46.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 39, and a V_{L} comprising an LC-CDR1, an LC-CDR2 and an LC-CDR3 of the V_{L} comprising the amino acid sequence of SEQ ID NO: 47.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 39, and a V_{L} comprising an LC-CDR1, an LC-CDR2 and an LC-CDR3 of the V_{L} comprising the amino acid sequence of SEQ ID NO: 48.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 39, and a V_{L} comprising an LC-CDR1, an LC-CDR2 and an LC-CDR3 of the V_{L} comprising the amino acid sequence of SEQ ID NO: 46.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 36, and a V_{L} comprising an LC-CDR1, an LC-CDR2 and an LC-CDR3 of the V_{L} comprising the amino acid sequence of SEQ ID NO: 47.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 36, and a V_{L} comprising an LC-CDR1, an LC-CDR2 and an LC-CDR3 of the V_{L} comprising the amino acid sequence of SEQ ID NO: 48.

In some embodiments, the anti-PD-L1 antibody comprises a V_{H} comprising the amino acid sequence of any one of SEQ ID NOs: 35-39, or a variant thereof having at least about 80% (such as at least about any of 80%, 85%, 90%, 95%, 96%, 97%, 98%, or 99%) sequence identity, and a V_{L} comprising the amino acid sequence of any one of SEQ ID NOs: 45-48, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of any one of SEQ ID NOs: 35-39, and a V_{L} comprising the amino acid sequence of any one of SEQ ID NOs: 45-48.

In some embodiments, the anti-PD-L1 antibody comprises a V_{H} comprising the amino acid sequence of SEQ ID NO: 35, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 45, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 35 and a V_{L} comprising the amino acid sequence of SEQ ID NO: 45.

In some embodiments, the anti-PD-L1 antibody comprises a V_{H} comprising the amino acid sequence of SEQ ID NO: 36, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 46, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 36 and a V_{L} comprising the amino acid sequence of SEQ ID NO: 46.

In some embodiments, the anti-PD-L1 antibody comprises a V_{H} comprising the amino acid sequence of SEQ ID NO: 37, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 47, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 37 and a V_{L} comprising the amino acid sequence of SEQ ID NO: 47.

In some embodiments, the anti-PD-L1 antibody comprises a V_{H} comprising the amino acid sequence of SEQ ID NO: 37, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 48, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 37 and a V_{L} comprising the amino acid sequence of SEQ ID NO: 48.

In some embodiments, the anti-PD-L1 antibody comprises a V_{H} comprising the amino acid sequence of SEQ ID NO: 37, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 46, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 37 and a V_{L} comprising the amino acid sequence of SEQ ID NO: 46.

In some embodiments, the anti-PD-L1 antibody comprises a V_{H} comprising the amino acid sequence of SEQ ID NO: 38, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 47, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 38 and a V_{L} comprising the amino acid sequence of SEQ ID NO: 47.

In some embodiments, the anti-PD-L1 antibody comprises a V_{H} comprising the amino acid sequence of SEQ ID NO: 38, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 48, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 38 and a V_{L} comprising the amino acid sequence of SEQ ID NO: 48.

In some embodiments, the anti-PD-L1 antibody comprises a V_{H} comprising the amino acid sequence of SEQ ID NO: 38, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 46, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 38 and a V_{L} comprising the amino acid sequence of SEQ ID NO: 46.

In some embodiments, the anti-PD-L1 antibody comprises a V_{H} comprising the amino acid sequence of SEQ ID NO: 39, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 47, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 39 and a V_{L} comprising the amino acid sequence of SEQ ID NO: 47.

In some embodiments, the anti-PD-L1 antibody comprises a V_{H} comprising the amino acid sequence of SEQ ID NO: 39, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 48, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 39 and a V_{L} comprising the amino acid sequence of SEQ ID NO: 48.

In some embodiments, the anti-PD-L1 antibody comprises a V_{H} comprising the amino acid sequence of SEQ ID NO: 39, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 46, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 39 and a V_{L} comprising the amino acid sequence of SEQ ID NO: 46.

In some embodiments, the anti-PD-L1 antibody comprises a V_{H} comprising the amino acid sequence of SEQ ID NO: 36, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 47, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 36 and a V_{L} comprising the amino acid sequence of SEQ ID NO: 47.

In some embodiments, the anti-PD-L1 antibody comprises a V_{H} comprising the amino acid sequence of SEQ ID NO: 36, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 48, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 36 and a V_{L} comprising the amino acid sequence of SEQ ID NO: 48.

In some embodiments, the anti-PD-L1 antibody comprises a V_{H} comprising: an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 12, or a variant thereof comprising up to about 5 amino acid substitutions in the HC-CDRs; and a V_{L} comprising an LC-CDR1 comprising the amino acid sequence of SEQ ID NO: 18, an LC-CDR2 comprising the amino acid sequence of SEQ ID NO: 25, and an LC-CDR3 comprising the amino acid sequence of SEQ ID NO: 30, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 12; and a V_{L} comprising an LC-CDR1 comprising the amino acid sequence of SEQ ID NO: 18, an LC-CDR2 comprising the amino acid sequence of SEQ ID NO: 25, and an LC-CDR3 comprising the amino acid sequence of SEQ ID NO: 30.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 40, and a V_{L} comprising an LC-CDR1, an LC-CDR2 and an LC-CDR3 of the V_{L} comprising the amino acid sequence of SEQ ID NO: 49.

In some embodiments, the anti-PD-L1 antibody comprises a V_{H} comprising the amino acid sequence of SEQ ID NO: 40, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 49, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 40 and a V_{L} comprising the amino acid sequence of SEQ ID NO: 49.

In some embodiments, the anti-PD-L1 antibody comprises a V_{H} comprising: an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 13, or a variant thereof comprising up to about 5 amino acid substitutions in the HC-CDRs; and a V_{L} comprising an LC-CDR1 comprising the amino acid sequence of SEQ ID NO: 19, an LC-CDR2 comprising the amino acid sequence of SEQ ID NO: 25, and an LC-CDR3 comprising the amino acid sequence of SEQ ID NO: 30, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 13; and a V_{L} comprising an LC-CDR1 comprising the amino acid sequence of SEQ ID NO: 19, an LC-CDR2 comprising the amino acid sequence of SEQ ID NO: 25, and an LC-CDR3 comprising the amino acid sequence of SEQ ID NO: 30.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 41, and a V_{L} comprising an LC-CDR1, an LC-CDR2 and an LC-CDR3 of the V_{L} comprising the amino acid sequence of SEQ ID NO: 50.

In some embodiments, the anti-PD-L1 antibody comprises a V_{H} comprising the amino acid sequence of SEQ ID NO: 41, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 50, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 41 and a V_{L} comprising the amino acid sequence of SEQ ID NO: 50.

In some embodiments, the anti-PD-L1 antibody comprises a V_{H} comprising: an HC-CDR1 comprising the amino acid sequence of SEQ ID NO: 3, an HC-CDR2 comprising the amino acid sequence of SEQ ID NO: 8, and an HC-CDR3 comprising the amino acid sequence of SEQ ID NO: 14, or a variant thereof comprising up to about 5 amino acid substitutions in the HC-CDRs; and a V_{L} comprising an LC-CDR1 comprising the amino acid sequence of SEQ ID NO: 20, an LC-CDR2 comprising the amino acid sequence of SEQ ID NO: 26, and an LC-CDR3 comprising the amino acid sequence of SEQ ID NO: 31, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 3, an HC-CDR2 comprising the amino acid sequence of SEQ ID NO: 8, and an HC-CDR3 comprising the amino acid sequence of SEQ ID NO: 14; and a V_{L} comprising an LC-CDR1 comprising the amino acid sequence of SEQ ID NO: 20, an LC-CDR2 comprising the amino acid sequence of SEQ ID NO: 26, and an LC-CDR3 comprising the amino acid sequence of SEQ ID NO: 31.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 42, and a V_{L} comprising an LC-CDR1, an LC-CDR2 and an LC-CDR3 of the V_{L} comprising the amino acid sequence of SEQ ID NO: 51.

In some embodiments, the anti-PD-L1 antibody comprises a V_{H} comprising the amino acid sequence of SEQ ID NO: 42, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 51, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 42 and a V_{L} comprising the amino acid sequence of SEQ ID NO: 51.

In some embodiments, the anti-PD-L1 antibody comprises a V_{H} comprising: an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15, or a variant thereof comprising up to about 5 amino acid substitutions in the HC-CDRs; and a V_{L} comprising an LC-CDR1 comprising the amino acid sequence of SEQ ID NO: 21, an LC-CDR2 comprising the amino acid sequence of SEQ ID NO: 26, and an LC-CDR3 comprising the amino acid sequence of SEQ ID NO: 32, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15; and a V_{L} comprising an LC-CDR1 comprising the amino acid sequence of SEQ ID NO: 21, an LC-CDR2 comprising the amino acid sequence of SEQ ID NO: 26, and an LC-CDR3 comprising the amino acid sequence of SEQ ID NO: 32.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 43, and a V_{L} comprising an LC-CDR1, an LC-CDR2 and an LC-CDR3 of the V_{L} comprising the amino acid sequence of SEQ ID NO: 52.

In some embodiments, the anti-PD-L1 antibody comprises a V_{H} comprising the amino acid sequence of SEQ ID NO: 43, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 52, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 43 and a V_{L} comprising the amino acid sequence of SEQ ID NO: 52.

In some embodiments, the anti-PD-L1 antibody comprises a V_{H} comprising: an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15, or a variant thereof comprising up to about 5 amino acid substitutions in the HC-CDRs; and a V_{L} comprising an LC-CDR1 comprising the amino acid sequence of SEQ ID NO: 22, an LC-CDR2 comprising the amino acid sequence of SEQ ID NO: 27, and an LC-CDR3 comprising the amino acid sequence of SEQ ID NO: 33, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15; and a V_{L} comprising an LC-CDR1 comprising the amino acid sequence of SEQ ID NO: 22, an LC-CDR2 comprising the amino acid sequence of SEQ ID NO: 27, and an LC-CDR3 comprising the amino acid sequence of SEQ ID NO: 33.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 43, and a V_{L} comprising an LC-CDR1, an LC-CDR2 and an LC-CDR3 of the V_{L} comprising the amino acid sequence of SEQ ID NO: 53.

In some embodiments, the anti-PD-L1 antibody comprises a V_{H} comprising the amino acid sequence of SEQ ID NO: 43, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 53, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 43 and a V_{L} comprising the amino acid sequence of SEQ ID NO: 53.

In some embodiments, the anti-PD-L1 antibody comprises a V_{H} comprising: an HC-CDR1 comprising the amino acid sequence of SEQ ID NO: 5, an HC-CDR2 comprising the amino acid sequence of SEQ ID NO: 10, and an HC-CDR3 comprising the amino acid sequence of SEQ ID NO: 16, or a variant thereof comprising up to about 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 28, and an LC-CDR3 comprising the amino acid sequence of SEQ ID NO: 34, or a variant thereof comprising up to about 5 amino acid substitutions in the LC-CDRs.

In some embodiments, the anti-PD-L1 antibody comprises a V_{H} comprising: an HC-CDR1 comprising the amino acid sequence of SEQ ID NO: 5, an HC-CDR2 comprising the amino acid sequence of SEQ ID NO: 10, and an HC-CDR3 comprising the amino acid sequence of SEQ ID NO: 16; and a V_{L} comprising an LC-CDR1 comprising the amino acid sequence of SEQ ID NO: 23, an LC-CDR2 comprising the amino acid sequence of SEQ ID NO: 28, and an LC-CDR3 comprising the amino acid sequence of SEQ ID NO: 34.

In some embodiments, the anti-PD-L1 antibody comprises a V_{H} comprising an HC-CDR1, an HC-CDR2 and an HC-CDR3 of the V_{H} comprising the amino acid sequence of SEQ ID NO: 44, and a V_{L} comprising an LC-CDR1, an LC-CDR2 and an LC-CDR3 of the V_{L} comprising the amino acid sequence of SEQ ID NO: 54.

In some embodiments, the anti-PD-L1 antibody comprises a V_{H} comprising the amino acid sequence of SEQ ID NO: 44, or a variant thereof having at least about 80% (such as at least about any of 80%, 85%, 90%, 95%, 96%, 97%, 98%, or 99%) sequence identity, and a V_{L} comprising the amino acid sequence of SEQ ID NO: 54, or a variant thereof having at least about 80% (such as at least about any of 80%, 85%, 90%, 95%, 96%, 97%, 98%, or 99%) sequence identity. In some embodiments, the anti-PD-L1 antibody comprises a V_{H} comprising the amino acid sequence of SEQ ID NO: 44 and a V_{L} comprising the amino acid sequence of SEQ ID NO: 54.

In some embodiments, the amino acid substitutions described above are limited to “exemplary substitutions” shown in Table 4 of this application. In some embodiments, the amino acid substitutions are limited to “preferred substitutions” shown in Table 4 of this application.

In some embodiments, functional epitopes can be mapped by combinatorial alanine scanning. In this process, a combinatorial alanine-scanning strategy can be used to identify amino acids in the PD-L1 protein that are necessary for interaction with PD-L1 antibodies. In some embodiments, the epitope is conformational and crystal structure of anti-PD-L1 antibodies bound to PD-L1 may be employed to identify the epitopes.

In some embodiments, the present application provides antibodies which compete with any one of the PD-L1 antibodies described herein for binding to PD-L1. In some embodiments, the present application provides antibodies which compete with any one of the anti-PD-L1 antibodies provided herein for binding to an epitope on the PD-L1. In some embodiments, an anti-PD-L1 antibody is provided that binds to the same epitope as an anti-PD-L1 antibody comprising a V_{H} comprising the amino acid sequence of any one of SEQ ID NOs: 35-44, and a V_{L} comprising the amino acid sequence of any one of SEQ ID NOs: 45-54. In some embodiments, an anti-PD-L1 antibody is provided that specifically binds to PD-L1 competitively with an anti-PD-L1 antibody comprising a V_{H} comprising the amino acid sequence of any one of SEQ ID NOs: 35-44 and a V_{L} comprising the amino acid sequence of any one of SEQ ID NOs: 45-54.

In some embodiments, competition assays may be used to identify a monoclonal antibody that competes with an anti-PD-L1 antibody described herein for binding to PD-L1. Competition assays can be used to determine whether two antibodies bind to the same epitope by recognizing identical or sterically overlapping epitopes or one antibody competitively inhibits binding of another antibody to the antigen. In certain embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, N.Y.). Detailed exemplary methods for mapping an epitope to which an antibody binds are provided in Morris (1996) “Epitope Mapping Protocols”, in Methods in Molecular Biology vol. 66 (Humana Press, Totowa, N.J.). In some embodiments, two antibodies are said to bind to the same epitope if each blocks binding of the other by 5000 or more. In some embodiments, the antibody that competes with an anti-PD-L1 antibody described herein is a chimeric, humanized or human antibody.

Exemplary anti-PD-L1 antibody sequences are shown in Tables 2 and 3, wherein the CDR numbering is according to the EU index of IMGT. Those skilled in the art will recognize that many algorithms are known for prediction of CDR positions and for delimitation of antibody heavy chain and light chain variable regions. Anti-PD-L1 antibodies comprising CDRs, V_{H} and/or V_{L} sequences from antibodies described herein, but based on prediction algorithms other than those exemplified in the tables below, are within the scope of this invention.

TABLE 2
Exemplary anti-PD-L1 antibody CDR sequences.

| Antibody | | | |
|---|---|---|---|
| Name | HC-CDR1 | HC-CDR2 | HC-CDR3 |
| Mab 3-16 | GFTFGGFG | ITGDSSTI | VRGPPGTWAY |
| Hum-1 | (SEQ ID NO: 1) | (SEQ ID NO: 6) | (SEQ ID NO: 11) |
| Hum-2 | | | |
| Hum-3 | | | |
| Hum-4 | | | |
| Hum-5 | | | |
| Hum-6 | | | |
| Hum-7 | | | |
| Hum-8 | | | |
| Hum-9 | | | |
| Hum-10 | | | |
| Hum-11 | | | |
| Hum-12 | | | |
| Mab1-01 | GYSFSGYF | INPSNDDT | ARFGHASWYFDV |
| | (SEQ ID NO: 2) | (SEQ ID NO: 7) | (SEQ ID NO: 12) |
| Mab1-02 | GYSFSGYF | INPSNDDT | ARFGHASWYLDV |
| | (SEQ ID NO: 2) | (SEQ ID NO: 7) | (SEQ ID NO: 13) |
| Mab2-04 | GYTFTDYA | IIPSGDHT | ARGHGFHLYVMDY |
| | (SEQ ID NO: 3) | (SEQ ID NO: 8) | (SEQ ID NO: 14) |
| Mab3-17 | GDSITSGY | VSYSGST | ANYYGDNWPFPLDY |
| | (SEQ ID NO: 4) | (SEQ ID NO: 9) | (SEQ ID NO: 15) |
| Mab3-18 | GDSITSGY | VSYSGST | ANYYGDNWPFPLDY |
| | (SEQ ID NO: 4) | (SEQ ID NO: 9) | (SEQ ID NO: 15) |
| Mab3-14 | GYTFSMYY | INPSNGRT | TREISYDEFAY |
| | (SEQ ID NO: 5) | (SEQ ID NO: 10) | (SEQ ID NO: 16) |
| Antibody | | | |
| Name | LC-CDR1 | LC-CDR2 | LC-CDR3 |
| Mab 3-16 | ESVEFYGTTL | GAS | QQIRKVPWT |
| Hum-1 | (SEQ ID NO: 17) | (SEQ ID NO: 24) | (SEQ ID NO: 29) |
| Hum-2 | | | |
| Hum-3 | | | |
| Hum-4 | | | |
| Hum-5 | | | |
| Hum-6 | | | |
| Hum-7 | | | |
| Hum-8 | | | |
| Hum-9 | | | |
| Hum-10 | | | |
| Hum-11 | | | |
| Hum-12 | | | |
| Mab1-01 | SSVIY | DTS | QQWSTYPLT |
| | (SEQ ID NO: 18) | (SEQ ID NO: 25) | (SEQ ID NO: 30) |
| Mab1-02 | SSIIY | DTS | QQWSTYPLT |
| | (SEQ ID NO: 19) | (SEQ ID NO: 25) | (SEQ ID NO: 30) |
| Mab2-04 | QSIADY | YAS | QNGHSFPPT |
| | (SEQ ID NO: 20) | (SEQ ID NO: 26) | (SEQ ID NO: 31) |
| Mab3-17 | QSINGN | YAS | QNGHGFPYT |
| | (SEQ ID NO: 21) | (SEQ ID NO: 26) | (SEQ ID NO: 32) |
| Mab3-18 | KSLLHSNGNTY | RMS | MQHLEYPFT |
| | (SEQ ID NO: 22) | (SEQ ID NO: 27) | (SEQ ID NO: 33) |
| Mab3-14 | QSLVHSNGNTY | RVS | SQSAHLPYT |
| | (SEQ ID NO: 23) | (SEQ ID NO: 28) | (SEQ ID NO: 34) |

TABLE 3
Exemplary sequences.

| SEQ ID | | |
|---|---|---|
| NO | Description | Sequence |
| 35 | Mab 3-16 V_{H} | DVQLVESGGGLVQPGGSRKLSCAASGFTFGGFGMHWVRQAPE |
| | | KGLEWVAYITGDSSTIYYADRVRGRFTISRDNPRNILFLQMTSLG |
| | | SEDTAMYYCVRGPPGTWAYWGQGTLVTVSA |
| 36 | Hum-1 V_{H} | EVQLVESGGGLVQPGGSLRLSCAASGFTFGGFGMNWVRQAPG |
| | Hum-11 V_{H} | KGLEWVSYITGDSSTIYYADRVRGRFTISRDNAKNILYLQMNSL |
| | Hum-12 V_{H} | GAEDTAVYYCVRGPPGTWAYWGQGTLVTVSS |
| 37 | Hum-2 V_{H} | EVQLVESGGGLVQPGGSLRLSCAASGFTFGGFGMNWVRQAPG |
| | Hum-3 V_{H} | KGLEWVSYITGDSSTIYYADRVRGRFTISRDNAKNSLYLQMNSL |
| | Hum-4 V_{H} | RAEDTAVYYCVRGPPGTWAYWGQGTLVTVSS |
| 38 | Hum-5 V_{H} | EVQLVESGGGLVQPGGSLRLSCAASGFTFGGFGMHWVRQAPG |
| | Hum-6 V_{H} | KGLEWVSYITGDSSTIYYADRVRGRFTISRDNAKNSLYLQMNSL |
| | Hum-7 V_{H} | RAEDTAVYYCVRGPPGTWAYWGQGTLVTVSS |
| 39 | Hum-8 V_{H} | EVQLVESGGGLVQPGGSLRLSCAASGFTFGGFGMNWVRQAPG |
| | Hum-9 V_{H} | KGLEWVSYITGDSSTIYYADRVRGRFTISRDNAKNILYLQMNSL |
| | Hum-10 V_{H} | RAEDTAVYYCVRGPPGTWAYWGQGTLVTVSS |
| 40 | Mab1-01 V_{H} | EVQLQQSGPELVKPGASVKMSCKASGYSFSGYFLNWVKQSHG |
| | | KSLEWIGRINPSNDDTVYSQKFKDRATLSLDRSSGTVHMELRSL |
| | | TSEDSAVYYCARFGHASWYFDVWGAGTTVTVSS |
| 41 | Mab1-02 V_{H} | EVQLQQSGPELVKPGASVKMSCKASGYSFSGYFLNWVKQSHG |
| | | KSLEWIGRINPSNDDTVYSQKFKDRATLSLDRSSGTVHMELRSL |
| | | TSEDSAVYYCARFGHASWYLDVWGAGTTVTVSS |
| 42 | Mab2-04 V_{H} | QVQLQQSGAELARPGASVKMSCKASGYTFTDYAMHWIKQRPG |
| | | QGLEWIGFIIPSGDHTDYNQNFKDRATLTADKSSNTAYMQLTSL |
| | | TPDDSAVYFCARGHGFHLYVMDYWGQGTSVTVSS |
| 43 | Mab3-17 V_{H} | EVHLQESGPSLVKPSQTLSLTCSVTGDSITSGYWNWIRQFPGNK |
| | Mab3-18 V_{H} | LEYMGYVSYSGSTYYNPSLKSRISITRDTSKNQYYLQLSSVTTED |
| | | TATYYCANYYGDNWPFPLDYWGQGTSVIVSS |
| 44 | Mab3-14 V_{H} | QVQLQQSGAELVKPGTSVKLSCKASGYTFSMYYMFWVKQRPG |
| | | QGLEWIGQINPSNGRTNFNEKFKSKATLTVDKSSTTAYMQLSSL |
| | | TSEDSAVYYCTREISYDEFAYWGQGTLVTVSA |
| 45 | Mab 3-16 V_{L} | DIVLTQSPASLAVSLGQRATFSCRASESVEFYGTTLVQWYQQRP |
| | | GQPPKLLIYGASNVESGVPARFTGSGSGTDFSLNIHPVEEDDIAM |
| | | YFCQQIRKVPWTFGGGTKLEIR |
| 46 | Hum-1 V_{L} | EIVLTQSPATLSLSPGERATLSCRASESVEFYGTTLVQWYQQKP |
| | Hum-4 V_{L} | GQAPRLLIYGASNVESGIPARFSGSGSGTDFTLTISSLEPEDFAVY |
| | Hum-7 V_{L} | FCQQIRKVPWTFGQGTKVEIK |
| | Hum-10 V_{L} | |
| 47 | Hum-2 V_{L} | EIVLTQSPATLSLSPGERATLSCRASESVEFYGTTLVQWYQQKP |
| | Hum-5 V_{L} | GQAPRLLIYGASNVESGIPARFSGSGSGTDFTLTISSLEPEDFAVY |
| | Hum-8 V_{L} | YCQQIRKVPWTFGQGTKVEIK |
| | Hum-11 V_{L} | |
| 48 | Hum-3 V_{L} | EIVLTQSPATLSLSPGERATLSCRASESVEFYGTTLVQWYQQKP |
| | Hum-6 V_{L} | GQPPRLLIYGASNVESGIPARFSGSGSGTDFTLTISSLEPEDFAVY |
| | Hum-9 V_{L} | YCQQIRKVPWTFGQGTKVEIK |
| | Hum-12 V_{L} | |
| 49 | Mab1-01 V_{L} | QIVLTQSPAIMSASPGEKVTMTCSARSSVIYMSWYQQKPGSSPR |
| | | LLIYDTSHLASGVPVRFSGSGSGTFYSLIISRVEAEDAATYYCQQ |
| | | WSTYPLTFGAGTRLELN |
| 50 | Mab1-02 V_{L} | QIVLTQSPAIMSASPGEKVTMTCSARSSIIYMSWYQQKTGSSPRL |
| | | LIYDTSHLASGVPVRFSGSGSGTFFSLIISRMEAEDAATYYCQQW |
| | | STYPLTFGAGTRLELK |
| 51 | Mab2-04 V_{L} | DIVMTQSPATLSVTPGDGVSLSCRASQSIADYLHWYQQKSHESP |
| | | RLLIKYASQSISGIPSRFSGSGSGSDFTLSISSVEPEDVGVYFCQNG |
| | | HSFPPTFGTGTKLELK |
| 52 | Mab3-17 V_{L} | DIVMTQSPATLSVTPGDRVSLSCRASQSINGNLHWFQQKSHESP |
| | | RLLIKYASQSISGIPSRFSGSGSGSDFTLRINSVEPEDVGVYYCQN |
| | | GHGFPYTFGGGTKLEIK |
| 53 | Mab3-18 V_{L} | DIVMTQAAPSVPVTPGESVSISCRSNKSLLHSNGNTYLYWFLQR |
| | | PGQSPQLLIYRMSNLASGVPDRFSGSGSGTAFTLTISRVEAEDVG |
| | | VYYCMQHLEYPFTFGGGTKLEIE |
| 54 | Mab3-14 V_{L} | DVVMTQTPLSLPVSLGDQASISCRSSQSLVHSNGNTYLHWYLQ |
| | | KPGQSPKLLIYRVSNRFSGVPDRFSGSGSGTDFTLKISRVEAEDL |
| | | GIYFCSQSAHLPYTFGGGTKLEIK |
| 55 | IgG1 heavy | ASTKGPSVFPLAPSSKSTSGGTAALGCLVKDYFPEPVTVSWNSG |
| | chain constant | ALTSGVHTFPAVLQSSGLYSLSSVVTVPSSSLGTQTYICNVNHKP |
| | region | SNTKVDKKVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPGK |
| 56 | IgG4 heavy | ASTKGPSVFPLAPCSRSTSESTAALGCLVKDYFPEPVTVSWNSG |
| | chain constant | ALTSGVHTFPAVLQSSGLYSLSSVVTVPSSSLGTKTYTCNVDHK |
| | region | PSNTKVDKRVESKYGPPCPSCPAPEFLGGPSVFLFPPKPKDTLMI |
| | | SRTPEVTCVVVDVSQEDPEVQFNWYVDGVEVHNAKTKPREEQ |
| | | FNSTYRVVSVLTVLHQDWLNGKEYKCKVSNKGLPSSIEKTISKA |
| | | KGQPREPQVYTLPPSQEEMTKNQVSLTCLVKGFYPSDIAVEWES |
| | | NGQPENNYKTTPPVLDSDGSFFLYSRLTVDKSRWQEGNVFSCS |
| | | VMHEALHNHYTQKSLSLSLGK |
| 57 | Light chain | RTVAAPSVFIFPPSDEQLKSGTASVVCLLNNFYPREAKVQWKVD |
| | constant | NALQSGNSQESVTEQDSKDSTYSLSSTLTLSKADYEKHKVYACE |
| | region(kappa) | VTHQGLSSPVTKSFNRGEC |
| 58 | Light chain | GQPKAAPSVTLFPPSSEELQANKATLVCLISDFYPGAVTVAWKA |
| | constant | DSSPVKAGVETTTPSKQSNNKYAASSYLSLTPEQWKSHRSYSCQ |
| | region(lambda) | VTHEGSTVEKTVAPTECS |

### PD-1 Receptor

Programmed cell death protein 1 (PD-1, also referred to as CD279) is a 288-amino acid protein type I transmembrane protein that belongs to the immunoglobulin (Ig) superfamily. PD-1 consists of an Ig-V like extracellular domain, a short amino acid stalk, a transmembrane domain, and an intracellular domain including an immunoreceptor tyrosine-based inhibitory motif (ITIM) (Zhang et al. 2004; Keir et al. 2008). It was identified as a member of the Ig superfamily, which is involved in programed cell death (Ishida et al. 1992) and was eventually recognized as a member of the CD28-B7 family.

PD-1 is induced by T-cell receptor (TCR) activation and cytokine signaling via interleukin receptor sharing the common gamma chain, such as IL-2, IL-7, IL-15 and IL-21 (Okazaki et al. 2013) (Kinter et al. 2008). PD-1 expression can be induced by stimulation of various immune cells, including CD4+ and CD8+ T cells; NK, NKT, and B cells; macrophages; and several subsets of dendritic cells (DCs) (Agata et al. 1996; Yamazaki et al. 2002). Despite such broad expression, the functional contribution of PD-1-mediated regulation is mostly dominant in CD8+ T-cell responses and the effector phase of T-cell responses in peripheral tissues (Dong et al. 2004; Goldberg et al. 2007; Iwai et al. 2003; Keir et al. 2006). This is because PD-1-mediated inhibition is often overcome by high IL-2 and CD28 co-stimulation in the presence of APCs (Carter et al. 2002; Kuipers et al. 2006). PD-L1 expression on parenchymal tissue cells, rather than on hematopoietic immune cells, is heavily involved in the PD-1-mediated peripheral T-cell tolerance (Dong et al. 2004; Keir et al. 2006; Ritprajak et al. 2010).

PD-1 signaling is a key pathway involved in regulating the threshold of T-cell activation and affecting effector T-cell responses, differentiation of T cells, T-cell tolerance, T-cell exhaustion, and inflammation in the periphery. PD-1 deficiency demonstrated the role of PD-1 in autoimmunity by resulting in moderate splenomegaly and hyper B-cell activation stimulated with IgM (Nishimura et al. 1998). It also resulted in the development of a variety of strain- and organ-specific autoimmune diseases, such as lupus-like arthritis and glomerulonephritis, in the C57BL/6 background, as well as in dilated cardiomyopathy in BALB/c mice (Nishimura et al. 1999; Nishimura et al. 2001).

### PD-L1

Programed cell death 1 ligand 1 (PD-L1, also referred to as B7-H1, CD274) (Dong et al. 1999; Freeman et al. 2000) is a 290-amino acid protein type I transmembrane protein that belongs to the immunoglobulin (Ig) superfamily. PD-L1 contains an Ig-V and Ig-C-like extracellular domain, a transmembrane domain, and a short cytoplasmic tail domain that does not contain canonical signaling motifs (Dong et al. 1999; Keir et al. 2008; Lin et al. 2008).

PD-L1 is constitutively expressed on immune cells, such as T cells, B cells, macrophages, and dendritic cells (DCs), and is upregulated by any inflammatory stimulation (Yamazaki et al. 2002). In addition, PD-L1 is also expressed on a variety of nonhematopoietic cells including vascular endothelium, pancreatic islets, and placental syncytiotrophoblasts (Keir et al. 2008). Particularly, PD-L1 is physiologically expressed on the dorsal surface of the tongue, gingiva, and hard palate but not on other squamous epithelia in the masticatory mucosa in the oral cavity (Kang et al. 2017). Expression of the PD-L1 gene has been shown to be controlled by inflammatory signaling, consistent with the physiological role of the PD-1-PD-L1 axis in suppressing T cell activation. A number of soluble factors produced by immune cells have over the past years been described as inducers of PD-L1. In one of the first reports indicating that PD-L1 could be employed by tumor cells as a defense mechanism against T cell attack (Dong et al., 2002), regulation of PD-L1 by interferon-γ (IFN-γ) was described for various tumor types, healthy tissues, and immune cells, and this has been extended in further studies (Brown et al., 2003; de Kleijn et al., 2013; Kryczek et al., 2008; Mazanet and Hughes, 2002; Nakazawa et al., 2004; Schoop et al., 2004; Wintterle et al., 2003). IFN-γ is a proinflammatory cytokine that is abundantly produced by T cells upon activation and that is also produced by NK cells. PD-L1 expression is also induced by interleukin (IL-17) (Zhao et al., 2011), tumor necrosis factor alpha (TNFα) (Mazanet and Hughes, 2002), and vascular endothelial growth factor (VEGF). In addition, PD-L1 expression has been found in several murine and human cancers, including human lung, ovarian and colon carcinoma and various myelomas (Iwai et al. (2002) PNAS 99:12293-7; Ohigashi et al. (2005) Clin Cancer Res 11:2947-53).

Although regulatory roles of PD-1 in T- and B-cell-mediated immune responses have been reported, the addition of PD-1 to the CD28-B7 family did not occur until the identification of the PD-1 ligands, PD-L1 (B7-H1, CD274) and PD-L2 (B7-DC, CD273) (Latchman et al. 2001; Tseng et al. 2001). PD-L1 binding to PD-1 recruits the phosphatase SHP-2, which induces the dephosphorylation of the proximal TCR signaling, thereby resulting in the PD-1-mediated immune regulation (Chemnitz et al. 2004; Sheppard et al. 2004; Yokosuka et al. 2012). Interactions between PD-1 and PD-L1 promote T-cell tolerance by blocking the TCR-induced stop signal required for close T and APC interaction (Fife et al. 2009). PD-L1 has been suggested to play a role in tumor immunity by increasing apoptosis of antigen-specific T cell clones (Dong et al. (2002) Nat Med 8:793-800). It has also been suggested that PD-L1 might be involved in intestinal mucosal inflammation and inhibition of PD-L1 suppresses wasting disease associated with colitis (Kanai et al. (2003) J Immunol 171:4156-63). Studies using neutralizing antibodies against PD-1, PD-L1, and/or PD-L2 demonstrate the involvement of PD-1-PD-L1-mediated regulation in murine models of chronic diseases and peripheral tolerance (Ansari et al. 2003; Fife et al. 2006; Habicht et al. 2007; Salama et al. 2003; Tanaka et al. 2007).

### Full-Length Anti-PD-L1 Antibody

The anti-PD-L1 antibody in some embodiments is a full-length anti-PD-L1 antibody. In some embodiments, the full-length anti-PD-L1 antibody is an IgA, IgD, IgE, IgG, or IgM. In some embodiments, the full-length anti-PD-L1 antibody comprises IgG constant domains, such as constant domains of any one of IgG1, IgG2, IgG3, and IgG4 including variants thereof. In some embodiments, the full-length anti-PD-L1 antibody comprises a lambda light chain constant region. In some embodiments, the full-length anti-PD-L1 antibody comprises a kappa light chain constant region. In some embodiments, the full-length anti-PD-L1 antibody is a full-length human anti-PD-L1 antibody. In some embodiments, the full-length anti-PD-L1 antibody comprises an Fc sequence of a mouse immunoglobulin. In some embodiments, the full-length anti-PD-L1 antibody comprises an Fc sequence that has been altered or otherwise changed so that it has enhanced antibody dependent cellular cytotoxicity (ADCC) or complement dependent cytotoxicity (CDC) effector function.

Thus, for example, in some embodiments, there is provided a full-length anti-PD-L1 antibody comprising IgG1 constant domains, wherein the anti-PD-L1 antibody specifically binds to PD-L1. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG2 constant domains, wherein the anti-PD-L1 antibody specifically binds to PD-L1. In some embodiments, the IgG2 is human IgG2. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, there is provided a full-length anti-PD-L1 antibody comprising IgG3 constant domains, wherein the anti-PD-L1 antibody specifically binds to PD-L1. In some embodiments, the IgG3 is human IgG3. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody specifically binds to PD-L1. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of any one of SEQ ID NOs: 1-5, or a variant thereof comprising up to about 3 (such as about any of 1, 2, or 3) amino acid substitutions, an HC-CDR2 comprising the amino acid sequence of any one of SEQ ID NOs: 6-10, or a variant thereof comprising up to about 3 (such as about any of 1, 2, or 3) amino acid substitutions, and an HC-CDR3 comprising the amino acid sequence of any one of SEQ ID NOs: 11-16, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 17-23, or a variant thereof comprising up to about 3 (such as about any of 1, 2, or 3) amino acid substitutions, an LC-CDR2 comprising the amino acid sequence of any one of SEQ ID NOs: 24-28, or a variant thereof comprising up to about 3 (such as about any of 1, 2, or 3) amino acid substitutions, and an LC-CDR3 comprising the amino acid sequence of any one of SEQ ID NOs: 29-34, or a variant thereof comprising up to about 3 (such as about any of 1, 2, or 3) amino acid substitutions. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG2 constant domains, wherein the anti-PD-L1 antibody comprises a) a heavy chain variable domain comprising an HC-CDR1 comprising the amino acid sequence of any one of SEQ ID NOs: 1-5, or a variant thereof comprising up to about 3 (such as about any of 1, 2, or 3) amino acid substitutions, an HC-CDR2 comprising the amino acid sequence of any one of SEQ ID NOs: 6-10, or a variant thereof comprising up to about 3 (such as about any of 1, 2, or 3) amino acid substitutions, and an HC-CDR3 comprising the amino acid sequence of any one of SEQ ID NOs: 11-16, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 17-23, or a variant thereof comprising up to about 3 (such as about any of 1, 2, or 3) amino acid substitutions, an LC-CDR2 comprising the amino acid sequence of any one of SEQ ID NOs: 24-28, or a variant thereof comprising up to about 3 (such as about any of 1, 2, or 3) amino acid substitutions, and an LC-CDR3 comprising the amino acid sequence of any one of SEQ ID NOs: 29-34, or a variant thereof comprising up to about 3 (such as about any of 1, 2, or 3) amino acid substitutions. In some embodiments, the IgG2 is human IgG2. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, there is provided a full-length anti-PD-L1 antibody comprising IgG3 constant domains, wherein the anti-PD-L1 antibody comprises a) a heavy chain variable domain comprising an HC-CDR1 comprising the amino acid sequence of any one of SEQ ID NOs: 1-5, or a variant thereof comprising up to about 3 (such as about any of 1, 2, or 3) amino acid substitutions, an HC-CDR2 comprising the amino acid sequence of any one of SEQ ID NOs: 6-10, or a variant thereof comprising up to about 3 (such as about any of 1, 2, or 3) amino acid substitutions, and an HC-CDR3 comprising the amino acid sequence of any one of SEQ ID NOs: 11-16, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 17-23, or a variant thereof comprising up to about 3 (such as about any of 1, 2, or 3) amino acid substitutions, an LC-CDR2 comprising the amino acid sequence of any one of SEQ ID NOs: 24-28, or a variant thereof comprising up to about 3 (such as about any of 1, 2, or 3) amino acid substitutions, and an LC-CDR3 comprising the amino acid sequence of any one of SEQ ID NOs: 29-34, or a variant thereof comprising up to about 3 (such as about any of 1, 2, or 3) amino acid substitutions. In some embodiments, the IgG3 is human IgG3. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of any one of SEQ ID NOs: 1-5, or a variant thereof comprising up to about 3 (such as about any of 1, 2, or 3) amino acid substitutions, an HC-CDR2 comprising the amino acid sequence of any one of SEQ ID NOs: 6-10, or a variant thereof comprising up to about 3 (such as about any of 1, 2, or 3) amino acid substitutions, and an HC-CDR3 comprising the amino acid sequence of any one of SEQ ID NOs: 11-16, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 17-23, or a variant thereof comprising up to about 3 (such as about any of 1, 2, or 3) amino acid substitutions, an LC-CDR2 comprising the amino acid sequence of any one of SEQ ID NOs: 24-28, or a variant thereof comprising up to about 3 (such as about any of 1, 2, or 3) amino acid substitutions, and an LC-CDR3 comprising the amino acid sequence of any one of SEQ ID NOs: 29-34, or a variant thereof comprising up to about 3 (such as about any of 1, 2, or 3) amino acid substitutions. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of any one of SEQ ID NOs: 1-5, an HC-CDR2 comprising the amino acid sequence of any one of SEQ ID NOs: 6-10, and an HC-CDR3 comprising the amino acid sequence of any one of SEQ ID NOs: 11-16, or a variant thereof comprising up to about 5 (such as about any of 1, 2, 3, 4, or 5) amino acid substitutions in the HC-CDR sequences; and b) a light chain variable domain comprising an LC-CDR1 comprising the amino acid sequence of any one of SEQ ID NOs: 17-23, an LC-CDR2 comprising the amino acid sequence of any one of SEQ ID NOs: 24-28, and an LC-CDR3 comprising the amino acid sequence of any one of SEQ ID NOs: 29-34, or a variant thereof comprising up to about 5 (such as about any of 1, 2, 3, 4, or 5) amino acid substitutions in the LC-CDR sequences. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of any one of SEQ ID NOs: 1-5, an HC-CDR2 comprising the amino acid sequence of any one of SEQ ID NOs: 6-10, and an HC-CDR3 comprising the amino acid sequence of any one of SEQ ID NOs: 11-16, or a variant thereof comprising up to about 5 (such as about any of 1, 2, 3, 4, or 5) amino acid substitutions in the HC-CDR sequences; and b) a light chain variable domain comprising an LC-CDR1 comprising the amino acid sequence of any one of SEQ ID NOs: 17-23, an LC-CDR2 comprising the amino acid sequence of any one of SEQ ID NOs: 24-28, and an LC-CDR3 comprising the amino acid sequence of any one of SEQ ID NOs: 29-34, or a variant thereof comprising up to about 5 (such as about any of 1, 2, 3, 4, or 5) amino acid substitutions in the LC-CDR sequences. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of any one of SEQ ID NOs: 1-5, an HC-CDR2 comprising the amino acid sequence of any one of SEQ ID NOs: 6-10, and an HC-CDR3 comprising the amino acid sequence of any one of SEQ ID NOs: 11-16; and b) a light chain variable domain comprising an LC-CDR1 comprising the amino acid sequence of any one of SEQ ID NOs: 17-23, an LC-CDR2 comprising the amino acid sequence of any one of SEQ ID NOs: 24-28, and an LC-CDR3 comprising the amino acid sequence of any one of SEQ ID NOs: 29-34. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of any one of SEQ ID NOs: 1-5, an HC-CDR2 comprising the amino acid sequence of any one of SEQ ID NOs: 6-10, and an HC-CDR3 comprising the amino acid sequence of any one of SEQ ID NOs: 11-16; and b) a light chain variable domain comprising an LC-CDR1 comprising the amino acid sequence of any one of SEQ ID NOs: 17-23, an LC-CDR2 comprising the amino acid sequence of any one of SEQ ID NOs: 24-28, and an LC-CDR3 comprising the amino acid sequence of any one of SEQ ID NOs: 29-34. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11; and b) a light chain variable domain comprising an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 12; and b) a light chain variable domain comprising an LC-CDR1 comprising the amino acid sequence of SEQ ID NO: 18, an LC-CDR2 comprising the amino acid sequence of SEQ ID NO: 25, and an LC-CDR3 comprising the amino acid sequence of SEQ ID NO: 30. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 13; and b) a light chain variable domain comprising an LC-CDR1 comprising the amino acid sequence of SEQ ID NO: 19, an LC-CDR2 comprising the amino acid sequence of SEQ ID NO: 25, and an LC-CDR3 comprising the amino acid sequence of SEQ ID NO: 30. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 3, an HC-CDR2 comprising the amino acid sequence of SEQ ID NO: 8, and an HC-CDR3 comprising the amino acid sequence of SEQ ID NO: 14; and b) a light chain variable domain comprising an LC-CDR1 comprising the amino acid sequence of SEQ ID NO: 20, an LC-CDR2 comprising the amino acid sequence of SEQ ID NO: 26, and an LC-CDR3 comprising the amino acid sequence of SEQ ID NO: 31. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15; and b) a light chain variable domain comprising an LC-CDR1 comprising the amino acid sequence of SEQ ID NO: 21, an LC-CDR2 comprising the amino acid sequence of SEQ ID NO: 26, and an LC-CDR3 comprising the amino acid sequence of SEQ ID NO: 32. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15; and b) a light chain variable domain comprising an LC-CDR1 comprising the amino acid sequence of SEQ ID NO: 22, an LC-CDR2 comprising the amino acid sequence of SEQ ID NO: 27, and an LC-CDR3 comprising the amino acid sequence of SEQ ID NO: 33. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a) a heavy chain variable domain comprising an HC-CDR1 comprising the amino acid sequence of SEQ ID NO: 5, an HC-CDR2 comprising the amino acid sequence of SEQ ID NO: 10, and an HC-CDR3 comprising the amino acid sequence of SEQ ID NO: 16; and b) a light chain variable domain comprising an LC-CDR1 comprising the amino acid sequence of SEQ ID NO: 23, an LC-CDR2 comprising the amino acid sequence of SEQ ID NO: 28, and an LC-CDR3 comprising the amino acid sequence of SEQ ID NO: 34. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11; and b) a light chain variable domain comprising an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 12; and b) a light chain variable domain comprising an LC-CDR1 comprising the amino acid sequence of SEQ ID NO: 18, an LC-CDR2 comprising the amino acid sequence of SEQ ID NO: 25, and an LC-CDR3 comprising the amino acid sequence of SEQ ID NO: 30. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 13; and b) a light chain variable domain comprising an LC-CDR1 comprising the amino acid sequence of SEQ ID NO: 19, an LC-CDR2 comprising the amino acid sequence of SEQ ID NO: 25, and an LC-CDR3 comprising the amino acid sequence of SEQ ID NO: 30. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 3, an HC-CDR2 comprising the amino acid sequence of SEQ ID NO: 8, and an HC-CDR3 comprising the amino acid sequence of SEQ ID NO: 14; and b) a light chain variable domain comprising an LC-CDR1 comprising the amino acid sequence of SEQ ID NO: 20, an LC-CDR2 comprising the amino acid sequence of SEQ ID NO: 26, and an LC-CDR3 comprising the amino acid sequence of SEQ ID NO: 31. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15; and b) a light chain variable domain comprising an LC-CDR1 comprising the amino acid sequence of SEQ ID NO: 21, an LC-CDR2 comprising the amino acid sequence of SEQ ID NO: 26, and an LC-CDR3 comprising the amino acid sequence of SEQ ID NO: 32. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15; and b) a light chain variable domain comprising an LC-CDR1 comprising the amino acid sequence of SEQ ID NO: 22, an LC-CDR2 comprising the amino acid sequence of SEQ ID NO: 27, and an LC-CDR3 comprising the amino acid sequence of SEQ ID NO: 33. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a) a heavy chain variable domain comprising an HC-CDR1 comprising the amino acid sequence of SEQ ID NO: 5, an HC-CDR2 comprising the amino acid sequence of SEQ ID NO: 10, and an HC-CDR3 comprising the amino acid sequence of SEQ ID NO: 16; and b) a light chain variable domain comprising an LC-CDR1 comprising the amino acid sequence of SEQ ID NO: 23, an LC-CDR2 comprising the amino acid sequence of SEQ ID NO: 28, and an LC-CDR3 comprising the amino acid sequence of SEQ ID NO: 34. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of any one of SEQ ID NOs: 35-44, or a variant thereof having at least about 80% (such as at least about any of 80%, 85%, 90%, 95%, 96%, 97%, 98%, or 99%) sequence identity, and a light chain variable domain comprising the amino acid sequence of any one of SEQ ID NOs: 45-54, or a variant thereof having at least about 80% (such as at least about any of 80%, 85%, 90%, 95%, 96%, 97%, 98%, or 99%) sequence identity. In some embodiments, the IgG1 is human IgG1. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG2 constant domains, wherein the anti-PD-L1 antibody comprises a heavy chain variable domain comprising the amino acid sequence of any one of SEQ ID NOs: 35-44, or a variant thereof having at least about 80% (such as at least about any of 80%, 85%, 90%, 95%, 96%, 97%, 98%, or 99%) sequence identity, and a light chain variable domain comprising the amino acid sequence of any one of SEQ ID NOs: 45-54, or a variant thereof having at least about 80% (such as at least about any of 80%, 85%, 90%, 95%, 96%, 97%, 98%, or 99%) sequence identity. In some embodiments, the IgG2 is human IgG2. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, there is provided a full-length anti-PD-L1 antibody comprising IgG3 constant domains, wherein the anti-PD-L1 antibody comprises a heavy chain variable domain comprising the amino acid sequence of any one of SEQ ID NOs: 35-44, or a variant thereof having at least about 80% (such as at least about any of 80%, 85%, 90%, 95%, 96%, 97%, 98%, or 99%) sequence identity, and a light chain variable domain comprising the amino acid sequence of any one of SEQ ID NOs: 45-54, or a variant thereof having at least about 80% (such as at least about any of 80%, 85%, 90%, 95%, 96%, 97%, 98%, or 99%) sequence identity. In some embodiments, the IgG3 is human IgG3. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of any one of SEQ ID NOs: 35-44, or a variant thereof having at least about 80% (such as at least about any of 80%, 85%, 90%, 95%, 96%, 97%, 98%, or 99%) sequence identity, and a light chain variable domain comprising the amino acid sequence of any one of SEQ ID NOs: 45-54, or a variant thereof having at least about 80% (such as at least about any of 80%, 85%, 90%, 95%, 96%, 97%, 98%, or 99%) sequence identity. In some embodiments, the IgG4 is human IgG4. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of any one of SEQ ID NOs: 35-44, and a light chain variable domain comprising the amino acid sequence of any one of SEQ ID NOs: 45-54. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of any one of SEQ ID NOs: 35-44, and a light chain variable domain comprising the amino acid sequence of any one of SEQ ID NOs: 45-54. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 35 and a light chain variable domain comprising the amino acid sequence of SEQ ID NO: 45. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 36 and a light chain variable domain comprising the amino acid sequence of SEQ ID NO: 46. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 37 and a light chain variable domain comprising the amino acid sequence of SEQ ID NO: 47. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 37 and a light chain variable domain comprising the amino acid sequence of SEQ ID NO: 48. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 37 and a light chain variable domain comprising the amino acid sequence of SEQ ID NO: 46. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 38 and a light chain variable domain comprising the amino acid sequence of SEQ ID NO: 47. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 38 and a light chain variable domain comprising the amino acid sequence of SEQ ID NO: 48. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 38 and a light chain variable domain comprising the amino acid sequence of SEQ ID NO: 46. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 39 and a light chain variable domain comprising the amino acid sequence of SEQ ID NO: 47. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 39 and a light chain variable domain comprising the amino acid sequence of SEQ ID NO: 48. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 39 and a light chain variable domain comprising the amino acid sequence of SEQ ID NO: 46. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 36 and a light chain variable domain comprising the amino acid sequence of SEQ ID NO: 47. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 36 and a light chain variable domain comprising the amino acid sequence of SEQ ID NO: 48. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 40 and a light chain variable domain comprising the amino acid sequence of SEQ ID NO: 49. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 41 and a light chain variable domain comprising the amino acid sequence of SEQ ID NO: 50. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 42 and a light chain variable domain comprising the amino acid sequence of SEQ ID NO: 51. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 43 and a light chain variable domain comprising the amino acid sequence of SEQ ID NO: 52. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 43 and a light chain variable domain comprising the amino acid sequence of SEQ ID NO: 53. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG1 constant domains, wherein the anti-PD-L1 antibody comprises a heavy chain variable domain comprising the amino acid sequence of SEQ ID NO: 44 and a light chain variable domain comprising the amino acid sequence of SEQ ID NO: 54. In some embodiments, the heavy chain constant region comprises or consists of the amino acid sequence of SEQ ID NO: 55. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 55 and the light chain constant region comprises or consists of the amino acid sequence of SEQ ID NO: 57. In some embodiments, the heavy chain constant region comprises or consists of the amino acid sequence of SEQ ID NO: 55 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 35 and a light chain variable domain comprising the amino acid sequence of SEQ ID NO: 45. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 36 and a light chain variable domain comprising the amino acid sequence of SEQ ID NO: 46. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 37 and a light chain variable domain comprising the amino acid sequence of SEQ ID NO: 47. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 37 and a light chain variable domain comprising the amino acid sequence of SEQ ID NO: 48. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 37 and a light chain variable domain comprising the amino acid sequence of SEQ ID NO: 46. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 38 and a light chain variable domain comprising the amino acid sequence of SEQ ID NO: 47. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 38 and a light chain variable domain comprising the amino acid sequence of SEQ ID NO: 48. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 38 and a light chain variable domain comprising the amino acid sequence of SEQ ID NO: 46. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 39 and a light chain variable domain comprising the amino acid sequence of SEQ ID NO: 47. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 39 and a light chain variable domain comprising the amino acid sequence of SEQ ID NO: 48. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 39 and a light chain variable domain comprising the amino acid sequence of SEQ ID NO: 46. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 36 and a light chain variable domain comprising the amino acid sequence of SEQ ID NO: 47. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 36 and a light chain variable domain comprising the amino acid sequence of SEQ ID NO: 48. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 40 and a light chain variable domain comprising the amino acid sequence of SEQ ID NO: 49. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 41 and a light chain variable domain comprising the amino acid sequence of SEQ ID NO: 50. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 42 and a light chain variable domain comprising the amino acid sequence of SEQ ID NO: 51. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 43 and a light chain variable domain comprising the amino acid sequence of SEQ ID NO: 52. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 43 and a light chain variable domain comprising the amino acid sequence of SEQ ID NO: 53. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

In some embodiments, there is provided a full-length anti-PD-L1 antibody comprising IgG4 constant domains, wherein the anti-PD-L1 antibody comprises a heavy chain variable domain comprising the amino acid sequence of SEQ ID NO: 44 and a light chain variable domain comprising the amino acid sequence of SEQ ID NO: 54. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58. In some embodiments, the heavy chain constant region comprises or consists of the amino acid sequence of SEQ ID NO: 56 and the light chain constant region comprises or consists of the amino acid sequence of SEQ ID NO: 57. In some embodiments, the heavy chain constant region comprises or consists of the amino acid sequence of SEQ ID NO: 56 and the light chain constant region comprises or consists of the amino acid sequence of SEQ ID NO: 58.

### Binding Affinity

Binding affinity can be indicated by Kd, Koff, Kon, or Ka. The term “Koff”, as used herein, is intended to refer to the off-rate constant for dissociation of an antibody from the antibody/antigen complex, as determined from a kinetic selection set up. The term “Kon”, as used herein, is intended to refer to the on-rate constant for association of an antibody to the antigen to form the antibody/antigen complex. The term dissociation constant “Kd”, as used herein, refers to the dissociation constant of a particular antibody-antigen interaction, and describes the concentration of antigen required to occupy one half of all of the antibody-binding domains present in a solution of antibody molecules at equilibrium, and is equal to Koff/Kon. The measurement of Kd presupposes that all binding agents are in solution. In the case where the antibody is tethered to a cell wall, e.g., in a yeast expression system, the corresponding equilibrium rate constant is expressed as EC50, which gives a good approximation of Kd. The affinity constant, Ka, is the inverse of the dissociation constant, Kd.

The dissociation constant (Kd) is used as an indicator showing affinity of antibody moieties to antigens. For example, easy analysis is possible by the Scatchard method using antibodies marked with a variety of marker agents, as well as by using Biacore (made by Amersham Biosciences), analysis of biomolecular interactions by surface plasmon resonance, according to the user's manual and attached kit. The Kd value that can be derived using these methods is expressed in units of M. An antibody that specifically binds to a target may have a Kd of, for example, ≤10⁻⁷M, ≤10⁻⁸M, ≤10⁻⁹M≤10⁻¹⁰M, ≤10⁻¹M, ≤10⁻¹²M, or ≤10⁻¹³M.

Binding specificity of the antibody can be determined experimentally by methods known in the art. Such methods comprise, but are not limited to, Western blots, ELISA-, RIA-, ECL-, IRMA-, EIA-, BIAcore-tests and peptide scans.

In some embodiments, the anti-PD-L1 antibody specifically binds to a target PD-L1 with a Kd of about 10⁻⁷M to about 10⁻¹³M (such as about 10⁻⁷M to about 10⁻¹³M, about 10⁻⁸M to about 10⁻¹³M, about 10⁻⁹M to about 10⁻¹³M, or about 10⁻¹⁰M to about 10⁻¹²M). Thus in some embodiments, the Kd of the binding between the anti-PD-L1 antibody and PD-L1, is about 10⁻⁷M to about 10⁻¹³M, about 1×10⁻⁷M to about 5×10⁻¹³M, about 10⁻⁷M to about 10⁻¹²M, about 10⁻⁷M to about 10⁻¹M, about 10⁻⁷M to about 10⁻¹⁰M, about 10⁻⁷M to about 10⁻⁹M, about 10⁻⁸ M to about 10⁻¹³M, about 1×10⁻⁸M to about 5×10⁻¹³ M, about 10⁻⁸M to about 10⁻¹²M, about 10⁻⁸M to about 10⁻¹M, about 10⁻⁸ M to about 10⁻¹⁰ M, about 10⁻⁸M to about 10⁻⁹M, about 5×10⁻⁹M to about 1×10⁻¹³M, about 5×10⁻⁹M to about 1×10⁻¹²M, about 5×10⁻⁹M to about 1×10⁻¹M, about 5×10⁻⁹M to about 1×10⁻¹⁰M, about 10⁻⁹M to about 10⁻¹³M, about 10⁻⁹M to about 10⁻¹²M, about 10⁻⁹ M to about 10⁻¹M, about 10⁻⁹M to about 10⁻¹⁰M, about 5×10⁻¹⁰M to about 1×10⁻¹³M, about 5×10⁻¹⁰ M to about 1×10⁻¹²M, about 5×10⁻¹⁰M to about 1×10⁻¹M, about 10⁻¹⁰M to about 10⁻¹³ M, about 1×10⁻¹⁰ M to about 5×10⁻¹³M, about 1×10⁻¹⁰M to about 1×10⁻¹² M, about 1×10⁻¹⁰ M to about 5×10⁻¹² M, about 1×10⁻¹⁰ M to about 1×10⁻¹ M, about 10⁻¹¹ M to about 10⁻¹³M, about 1×10⁻¹ M to about 5×10⁻¹³ M, about 10⁻¹M to about 10⁻¹² M, or about 10⁻¹²M to about 10⁻¹³M. In some embodiments, the Kd of the binding between the anti-PD-L1 antibody and a PD-L1 is about 10⁻⁷M to about 10⁻¹³M.

In some embodiments, the Kd of the binding between the anti-PD-L1 antibody and a non-target is more than the Kd of the binding between the anti-PD-L1 antibody and the target, and is herein referred to in some embodiments as the binding affinity, of the anti-PD-L1 antibody to the target (e.g., PD-L1) is higher than that to a non-target. In some embodiments, the non-target is an antigen that is not PD-L1. In some embodiments, the Kd of the binding between the anti-PD-L1 antibody (against PD-L1) and a non-PD-L1 target can be at least about 10 times, such as about 10-100 times, about 100-1000 times, about 10³-10⁴ times, about 10⁴-10⁵ times, about 10⁵-10⁶ times, about 10⁶-10⁷ times, about 10⁷-10⁸ times, about 10⁸-10⁹ times, about 10⁹-10¹⁰ times, about 10¹⁰-10¹¹ times, or about 10¹¹-10¹² times of the Kd of the binding between the anti-PD-L1 antibody and a target PD-L1.

In some embodiments, the anti-PD-L1 antibody binds to a non-target with a Kd of about 10⁻¹M to about 10⁻⁶M (such as about 10⁻¹M to about 10⁻⁶M, about 10⁻¹M to about 10⁻⁵M, or about 10⁻²M to about 10⁻⁴M). In some embodiments, the non-target is an antigen that is not PD-L1. Thus in some embodiments, the Kd of the binding between the anti-PD-L1 antibody and a non-PD-L1 target is about 10⁻¹M to about 10⁻⁶M, about 1×10⁻¹M to about 5×10⁻⁶M, about 10⁻¹M to about 10⁻⁵M, about 1×10⁻¹M to about 5×10⁻⁵M, about 10⁻¹M to about 10⁻⁴M, about 1×10⁻¹M to about 5×10⁻⁴M, about 10⁻¹M to about 10⁻³M, about 1×10⁻¹M to about 5×10⁻³ M, about 10⁻¹M to about 10⁻²M, about 10⁻²M to about 10⁻⁶M, about 1×10⁻²M to about 5×10⁻⁶M, about 10⁻²M to about 10⁻⁵M, about 1×10⁻² M to about 5×10⁻⁵ M, about 10⁻²M to about 10⁻⁴M, about 1×10⁻²M to about 5×10⁻⁴ M, about 10⁻²M to about 10⁻³M, about 10⁻³M to about 10⁻⁶M, about 1×10⁻³ M to about 5×10⁻⁶M, about 10⁻³M to about 10⁻⁵M, about 1×10⁻³M to about 5×10⁻⁵M, about 10⁻³M to about 10⁻⁴M, about 10⁻⁴M to about 10⁻⁶M, about 1×10⁻⁴M to about 5×10⁻⁶M, about 10⁻⁴M to about 10⁻⁵M, or about 10⁻⁵M to about 10⁻⁶M.

In some embodiments, when referring to that the anti-PD-L1 antibody specifically recognizes a target PD-L1 at a high binding affinity, and binds to a non-target at a low binding affinity, the anti-PD-L1 antibody will bind to the target PD-L1 with a Kd of about 10⁻⁷M to about 10⁻¹³M (such as about 10⁻⁷M to about 10⁻¹³M, about 10⁻⁸M to about 10⁻¹³M, about 10⁻⁹M to about 10⁻¹³M, or about 10⁻¹⁰M to about 10⁻¹²M), and will bind to the non-target with a Kd of about 10⁻¹M to about 10⁻⁶M (such as about 10⁻¹M to about 10⁻⁶M, about 10⁻¹M to about 10⁻⁵M, or about 10⁻³M to about 10⁻⁴M).

In some embodiments, when referring to that the anti-PD-L1 antibody specifically recognizes PD-L1, the binding affinity of the anti-PD-L1 antibody is compared to that of a control anti-PD-L1 antibody (such as Ref). In some embodiments, the Kd of the binding between the control anti-PD-L1 antibody and PD-L1 can be at least about 2 times, such as about 2 times, about 3 times, about 4 times, about 5 times, about 6 times, about 7 times, about 8 times, about 9 times, about 10 times, about 10-100 times, about 100-1000 times, about 10³-10⁴ times of the Kd of the binding between the anti-PD-L1 antibody described herein and PD-L1.

### Nucleic Acids

Nucleic acid molecules encoding the anti-PD-L1 antibodies are also contemplated. In some embodiments, there is provided a nucleic acid (or a set of nucleic acids) encoding a full-length anti-PD-L1 antibody, including any of the full-length anti-PD-L1 antibodies described herein. In some embodiments, the nucleic acid (or a set of nucleic acids) encoding the anti-PD-L1 antibody described herein may further comprises a nucleic acid sequence encoding a peptide tag (such as protein purification tag, e.g., His-tag, HA tag).

Also contemplated here are isolated host cells comprising an anti-PD-L1 antibody, an isolated nucleic acid encoding the polypeptide components of the anti-PD-L1 antibody, or a vector comprising a nucleic acid encoding the polypeptide components of the anti-PD-L1 antibody described herein.

The present application also includes variants to these nucleic acid sequences. For example, the variants include nucleotide sequences that hybridize to the nucleic acid sequences encoding the anti-PD-L1 antibodies of the present application under at least moderately stringent hybridization conditions.

The present application also provides vectors in which a nucleic acid of the present application is inserted.

In brief summary, the expression of an anti-PD-L1 antibody (e.g., full-length anti-PD-L1 antibody) by a natural or synthetic nucleic acid encoding the anti-PD-L1 antibody can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequences.

The nucleic acids of the present application may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the application provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the application should not be limited to the use of constitutive promoters. Inducible promoters are also contemplated as part of the application. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-PD-L1 antibody is inducible. In some embodiments, a nucleic acid sequence encoding the anti-PD-L1 antibody is operably linked to an inducible promoter, including any inducible promoter described herein.

### Inducible Promoters

The use of an inducible promoter provides a molecular switch capable of turning on expression of the polynucleotide sequence to which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al. (1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405. In some embodiments, the inducible promoter system for use to express the anti-PD-L1 antibody is the Tet system. In some embodiments, the inducible promoter system for use to express the anti-PD-L1 antibody is the lac repressor system from E. coli.

An exemplary inducible promoter system for use in the present application is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a full-length anti-PD-L1 antibody according to any of the full-length anti-PD-L1 antibodies described herein. In some embodiments, the nucleic acid comprises one or more nucleic acid sequences encoding the heavy and light chains of the full-length anti-PD-L1 antibody. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present application, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the application.

### Preparation of Anti-PD-L1 Antibodies

In some embodiments, the anti-PD-L1 antibody is a monoclonal antibody or derived from a monoclonal antibody. In some embodiments, the anti-PD-L1 antibody comprises V_{H} and V_{L} domains, or variants thereof, from the monoclonal antibody. In some embodiments, the anti-PD-L1 antibody further comprises C_{H}1 and C_{L} domains, or variants thereof, from the monoclonal antibody. Monoclonal antibodies can be prepared, e.g., using known methods in the art, including hybridoma methods, phage display methods, or using recombinant DNA methods. Additionally, exemplary phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-PD-L1 antibodies described herein, the anti-PD-L1 antibody comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-PD-L1 antibodies can be prepared using phage display to screen libraries for anti-PD-L1 antibody moieties specific to the target PD-L1. The library can be a human scFv phage display library having a diversity of at least 1×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target PD-L1 with high affinity can be selected by iterative binding of phage to the target PD-L1, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL1-Blue, for expression and purification. The panning can be performed for multiple (such as about any of 2, 3, 4, 5, 6 or more) rounds with solution panning, cell panning, or a combination of both, to enrich for phage clones binding specifically to the target PD-L1. Enriched phage clones can be tested for specific binding to the target PD-L1 by any methods known in the art, including for example ELISA and FACS.

An alternative method for screening antibody libraries is to display the protein on the surface of yeast cells. Wittrup et al. (U.S. Pat. Nos. 6,699,658 and 6,696,251) have developed a method for a yeast cell display library. In this yeast display system, a component involves the yeast agglutinin protein (Aga1), which is anchored to the yeast cell wall. Another component involves a second subunit of the agglutinin protein Aga2, which can display on the surface yeast cells through disulfide bonds to Aga1 protein. The protein Aga1 is expressed from a yeast chromosome after the Aga1 gene integration. A library of single chain variable fragments (scFv) is fused genetically to Aga2 sequence in the yeast display plasmid, which, after transformation, is maintained in yeast episomally with a nutritional marker. Both of the Aga1 and Aga2 proteins were expressed under the control of the galactose-inducible promoter.

Human antibody V gene repertoire (V_{H} and V_{K} fragments) are obtained by PCR method using a pool of degenerate primers (Sblattero, D. & Bradbury, A. Immunotechnology 3, 271-278 1998). The PCR templates are from the commercially available RNAs or cDNAs, including PBMC, spleen, lymph nodes, bone marrow and tonsils. Separate V_{H} and V_{K} PCR libraries were combined, then assembled together in the scFv format by overlap extension PCR (Sheets, M. D. et al., Proc. Natl. Acad. Sci. USA 95, 6157-6162 1998). To construct the yeast scFv display library, the resultant scFv PCR products are cloned into the yeast display plasmid in the yeasts by homologous recombination. (Chao, G, et al., Nat Protoc. 2006; 1(2):755-68. Miller K D, et al., Current Protocols in Cytometry 4.7.1-4.7.30, 2008).

The anti-PD-L1 antibodies can be discovered using mammalian cell display systems in which antibody moieties are displayed on the cell surface and those specific to the target PD-L1 are isolated by the antigen-guided screening method, as described in U.S. Pat. No. 7,732,195B2. A Chinese hamster ovary (CHO) cell library representing a large set of human IgG antibody genes can be established and used to discover the clones expressing high-affinity antibody genes. Another display system has been developed to enable simultaneous high-level cell surface display and secretion of the same protein through alternate splicing, where the displayed protein phenotype remains linked to genotype, allowing soluble secreted antibody to be simultaneously characterized in biophysical and cell-based functional assays. This approach overcomes many limitations of previous mammalian cell display, enabling direct selection and maturation of antibodies in the form of full-length, glycosylated IgGs (Peter M. Bowers, et al., Methods 2014, 65:44-56). Transient expression systems are suitable for a single round of antigen selection before recovery of the antibody genes and therefore most useful for the selection of antibodies from smaller libraries. Stable episomal vectors offer an attractive alternative. Episomal vectors can be transfected at high efficiency and stably maintained at low copy number, permitting multiple rounds of panning and the resolution of more complex antibody libraries.

The IgG library is based on germline sequence V-gene segments joined to rearranged (D)J regions isolated from a panel of human donors. RNA collected from 2000 human blood samples was reverse-transcribed into cDNA, and the V_{H} and V_{K} fragments were amplified using V_{H}- and V_{K}-specific primers and purified by gel extraction. IgG libraries were generated by sub-cloning the V_{H} and V_{K} fragments into the display vectors containing IgG1 or K constant regions respectively and then electroporating into or transducing 293T cells. To generate the scFv antibody display library, scFvs were generated by linking V_{H} and V_{K}, and then sub-cloned into the display vector, which were then electroporated into or transduce 293T cells. As we known, the IgG library is based on germline sequence V-gene segments joined to rearranged (D)J regions isolated from a panel of donors, the donor can be a mouse, rat, rabbit, or monkey.

Monoclonal antibodies can also be made by recombinant DNA methods, such as those described in U.S. Pat. No. 4,816,567. DNA encoding the monoclonal antibodies of the application can be readily isolated and sequenced using conventional procedures (e.g., by using oligonucleotide probes that are capable of binding specifically to genes encoding the heavy and light chains of murine antibodies). Hybridoma cells as described above or PD-L1-specific phage clones of the application can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the application, or can be substituted for the variable domains of one antigen-combining site of an antibody of the application to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

### Human and Humanized Antibodies

The anti-PD-L1 antibodies (e.g., full-length anti-PD-L1 antibodies) can be humanized antibodies or human antibodies. Humanized forms of non-human (e.g., murine) antibody moieties are chimeric immunoglobulins, immunoglobulin chains, or fragments thereof (such as Fv, Fab, Fab′, F(ab′)2, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Anti-PD-L1 Antibody Variants

In some embodiments, amino acid sequences of the anti-PD-L1 antibody variants (e.g., full-length anti-PD-L1 antibody) provided herein are contemplated. For example, it may be desirable to improve the binding affinity and/or other biological properties of the antibody. Amino acid sequences of an antibody variant may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, anti-PD-L1 antibody variants having one or more amino acid substitutions are provided. Sites of interest for substitutional mutagenesis include the HVRs and FRs. Amino acid substitutions may be introduced into an antibody of interest and the products screened for a desired activity, e.g., improved bioactivity, retained/improved antigen binding, decreased immunogenicity, or improved ADCC or CDC.

Conservative substitutions are shown in Table 4 below.

TABLE 4
CONSERVATIVE SUBSTITUTIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody, which may be conveniently generated, e.g., using phage display-based affinity maturation techniques. Briefly, one or more CDR residues are mutated and the variant antibody moieties displayed on phage and screened for a particular biological activity (e.g., bioactivity based on mixed lymphocyte reaction (MLR) assay or binding affinity). Alterations (e.g., substitutions) may be made in HVRs, e.g., to improve bioactivity based on mixed lymphocyte reaction (MLR) assay or binding affinity. Such alterations may be made in HVR “hotspots”, e.g., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} and V_{L} being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001)).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., Ala or Glu) to determine whether the interaction of the antibody with antigen is affected. Further substitutions may be introduced at the amino acid locations to demonstrate functional sensitivity to the initial substitutions. Alternatively, or additionally, a crystal structure of an antigen-antibody complex can be determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Fc Region Variants

In some embodiments, one or more amino acid modifications may be introduced into the Fc region of an antibody (e.g., a full-length anti-PD-L1 antibody or anti-PD-L1 Fc fusion protein) provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al., J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-PD-L1 antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the application contemplates an anti-PD-L1 antibody variant (such as a full-length anti-PD-L1 antibody variant) comprising an Fc region that possesses some but not all effector functions, which makes it a desirable candidate for applications in which the half-life of the anti-PD-L1 antibody in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al., Proc. Nat'l Acad. Sci. USA 83:7059-7063 (1986)) and Hellstrom, I. et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, there is provided an anti-PD-L1 antibody (such as a full-length anti-PD-L1 antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-PD-L1 antibody (e.g., full-length anti-PD-L1 antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-PD-L1 antibody (such as a full-length anti-PD-L1 antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-PD-L1 antibodies (such as full-length anti-PD-L1 antibodies) comprising any of the Fc variants described herein, or combinations thereof, are contemplated.

### Glycosylation Variants

In some embodiments, an anti-PD-L1 antibody (such as a full-length anti-PD-L1 antibody) provided herein is altered to increase or decrease the extent to which the anti-PD-L1 antibody is glycosylated. Addition or deletion of glycosylation sites to an anti-PD-L1 antibody may be conveniently accomplished by altering the amino acid sequence of the anti-PD-L1 antibody or polypeptide portion thereof such that one or more glycosylation sites are created or removed.

Wherein the anti-PD-L1 antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-PD-L1 antibody of the application may be made in order to create anti-PD-L1 antibody variants with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-PD-L1 antibody (such as a full-length anti-PD-L1 antibody) variants are provided comprising an Fc region wherein a carbohydrate structure attached to the Fc region has reduced fucose or lacks fucose, which may improve ADCC function. Specifically, anti-PD-L1 antibodies are contemplated herein that have reduced fucose relative to the amount of fucose on the same anti-PD-L1 antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-PD-L1 antibody is one wherein less than about 50%, 40%, 30%, 20%, 10%, or 5% of the N-linked glycans thereon comprise fucose. For example, the amount of fucose in such an anti-PD-L1 antibody may be from 1% to 80%, from 1% to 65%, from 5% to 65% or from 20% to 40%. In some embodiments, the anti-PD-L1 antibody is one wherein none of the N-linked glycans thereon comprise fucose, i.e., wherein the anti-PD-L1 antibody is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng. 94(4):680-688 (2006); and WO2003/085107).

Anti-PD-L1 antibody (such as a full-length anti-PD-L1 antibody) variants are further provided with bisected oligosaccharides, e.g., in which a biantennary oligosaccharide attached to the Fc region of the anti-PD-L1 antibody is bisected by GlcNAc. Such anti-PD-L1 antibody (such as a full-length anti-PD-L1 antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-PD-L1 antibody (such as full-length anti-PD-L1 antibody) variants with at least one galactose residue in the oligosaccharide attached to the Fc region are also provided. Such anti-PD-L1 antibody variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-PD-L1 antibody (such as a full-length anti-PD-L1 antibody) variants comprising an Fc region are capable of binding to an FcγRIII. In some embodiments, the anti-PD-L1 antibody (such as a full-length anti-PD-L1 antibody) variants comprising an Fc region have ADCC activity in the presence of human effector cells (e.g., T cell) or have increased ADCC activity in the presence of human effector cells compared to the otherwise same anti-PD-L1 antibody (such as a full-length anti-PD-L1 antibody) comprising a human wild-type IgG1Fc region.

### Cysteine Engineered Variants

In some embodiments, it may be desirable to create cysteine engineered anti-PD-L1 antibodies (such as a full-length anti-PD-L1 antibody) in which one or more amino acid residues are substituted with cysteine residues. In some embodiments, the substituted residues occur at accessible sites of the anti-PD-L1 antibody. By substituting those residues with cysteine, reactive thiol groups are thereby positioned at accessible sites of the anti-PD-L1 antibody and may be used to conjugate the anti-PD-L1 antibody to other moieties, such as drug moieties or linker-drug moieties, to create an anti-PD-L1 immunoconjugate, as described further herein. Cysteine engineered anti-PD-L1 antibodies (e.g., full-length anti-PD-L1 antibodies) may be generated as described, e.g., in U.S. Pat. No. 7,521,541.

### Derivatives

In some embodiments, an anti-PD-L1 antibody (such as a full-length anti-PD-L1 antibody) provided herein may be further modified to contain additional non-proteinaceous moieties that are known in the art and readily available. The moieties suitable for derivatization of the anti-PD-L1 antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-PD-L1 antibody may vary, and if more than one polymer are attached, they can be the same or different molecules. In general, the number and/or type of polymers used for derivatization can be determined based on considerations including, but not limited to, the particular properties or functions of anti-PD-L1 antibody to be improved, whether the anti-PD-L1 antibody derivative will be used in a therapy under defined conditions, etc.

### Pharmaceutical Compositions

Also provided herein are compositions (such as pharmaceutical compositions, also referred to herein as formulations) comprising any of the anti-PD-L1 antibodies (such as a full-length anti-PD-L1 antibody), nucleic acids encoding the antibodies, vectors comprising the nucleic acids encoding the antibodies, or host cells comprising the nucleic acids or vectors described herein. In some embodiments, there is provided a pharmaceutical composition comprising any one of the anti-PD-L1 antibodies described herein and a pharmaceutically acceptable carrier.

Suitable formulations of the anti-PD-L1 antibodies are obtained by mixing an anti-PD-L1 antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-PD-L1 antibodies of this application into cells.

The formulation herein may also contain one or more active compounds in addition to the anti-PD-L1 antibody (such as a full-length anti-PD-L1 antibody) as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-PD-L1 antibody. Such molecules are suitably present in combination in amounts that are effective for the purpose intended. The effective amount of such other agents depends on the amount of anti-PD-L1 antibody present in the formulation, the type of disease or disorder or treatment, and other factors discussed above. These are generally used in the same dosages and with administration routes as described herein or about from 1 to 99% of the heretofore employed dosages.

The anti-PD-L1 antibodies (e.g., full-length anti-PD-L1 antibodies) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-PD-L1 antibodies (e.g., full-length anti-PD-L1 antibodies) can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D (−)-3-hydroxybutyric acid. While polymers such as ethylene-vinyl acetate and lactic acid-glycolic acid enable release of molecules for over 100 days, certain hydro gels release proteins for shorter time periods. When encapsulated antibody remain in the body for a long time, they can denature or aggregate as a result of exposure to moisture at 37° C., resulting in a loss of biological activity and possible changes in immunogenicity. Rational strategies can be devised for stabilization of anti-PD-L1 antibodies depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-PD-L1 antibody (such as a full-length anti-PD-L1 antibody) is formulated in a buffer comprising a citrate, NaCl, acetate, succinate, glycine, polysorbate 80 (Tween 80), or any combination of the foregoing.

The formulations to be used for in vivo administration must be sterile. This is readily accomplished by, e.g., filtration through sterile filtration membranes.

### Methods of Treatment Using Anti-PD-L1 Antibodies

The anti-PD-L1 antibodies (e.g., full-length anti-PD-L1 antibodies) and/or compositions of the application can be administered to individuals (e.g., mammals such as humans) to treat a disease and/or disorder associated with the activity and/or expression of PD-L1 or a T-cell dysfunction disorder (e.g., cancer or infectious disease). These diseases include, but are not limited to, melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. The present application thus in some embodiments provides a method of treating a disease or condition associated with the activity and/or expression of PD-L1 or a T-cell dysfunction disorder (e.g., cancer or infectious disease) in an individual comprising administering to the individual an effective amount of a composition (such as a pharmaceutical composition) comprising an anti-PD-L1 antibody (e.g., a full-length anti-PD-L1 antibody), such as any one of the anti-PD-L1 antibodies (e.g., full-length anti-PD-L1 antibodies) described herein. In some embodiments, the individual is human.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) specifically binding to an epitope on human PD-L1, wherein the epitope comprises amino acid residues of human PD-L1. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a heavy chain variable domain (V_{H}) comprising a heavy chain complementarity determining region (HC-CDR) 1 comprising GFTFGGFG (SEQ ID NO: 1); an HC-CDR2 comprising ITGDSSTI (SEQ ID NO: 6); and an HC-CDR3 comprising VRGPPGTWAY (SEQ ID NO: 11); and a light chain variable domain (V_{L}) comprising a light chain complementarity determining region (LC-CDR) 1 comprising ESVEFYGTTL (SEQ ID NO: 17); an LC-CDR2 comprising GAS (SEQ ID NO: 24); and an LC-CDR3 comprising QQIRKVPWT (SEQ ID NO: 29). In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising: a V_{H} comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 11, or a variant thereof comprising up to 5 amino acid substitutions in the HC-CDRs; and a V_{L} comprising an LC-CDR1 comprising the amino acid sequence of SEQ ID NO: 17, an LC-CDR2 comprising the amino acid sequence of SEQ ID NO: 24, and an LC-CDR3 comprising the amino acid sequence of SEQ ID NO: 29, or a variant thereof comprising up to 5 amino acid substitutions in the LC-CDRs.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of any one of SEQ ID NOs: 35-39 or a variant thereof having at least about 80% sequence identity to the amino acid sequence of any one of SEQ ID NOs: 35-39, and a V_{L} comprising the amino acid sequence of any one of SEQ ID NOs: 45-48, or a variant thereof having at least about 80% sequence identity to the amino acid sequence of any one of SEQ ID NOs: 45-48.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 35 and a V_{L} comprising the amino acid sequence of SEQ ID NO: 45. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 36 and a V_{L} comprising the amino acid sequence of SEQ ID NO: 46. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 37 and a V_{L} comprising the amino acid sequence of SEQ ID NO: 47. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 37 and a V_{L} comprising the amino acid sequence of SEQ ID NO: 48. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 37 and a V_{L} comprising the amino acid sequence of SEQ ID NO: 46. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 38 and a V_{L} comprising the amino acid sequence of SEQ ID NO: 47. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 38 and a V_{L} comprising the amino acid sequence of SEQ ID NO: 48. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 38 and a V_{L} comprising the amino acid sequence of SEQ ID NO: 46. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 39 and a V_{L} comprising the amino acid sequence of SEQ ID NO: 47. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 39 and a V_{L} comprising the amino acid sequence of SEQ ID NO: 48. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 39 and a V_{L} comprising the amino acid sequence of SEQ ID NO: 46. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 36 and a V_{L} comprising the amino acid sequence of SEQ ID NO: 47. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the anti-PD-L1 antibody provided herein comprises a V_{H} comprising the amino acid sequence of SEQ ID NO: 36 and a V_{L} comprising the amino acid sequence of SEQ ID NO: 48. In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 12, or a variant thereof comprising up to 5 amino acid substitutions in the HC-CDRs; and a V_{L} comprising an LC-CDR1 comprising the amino acid sequence of SEQ ID NO: 18, an LC-CDR2 comprising the amino acid sequence of SEQ ID NO: 25, and an LC-CDR3 comprising the amino acid sequence of SEQ ID NO: 30, or a variant thereof comprising up to 5 amino acid substitutions in the LC-CDRs. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of SEQ ID NO: 40 or a variant thereof having at least about 80% sequence identity to the amino acid sequence of SEQ ID NO: 40, and a V_{L} comprising the amino acid sequence of SEQ ID NO: 49, or a variant thereof having at least about 80% sequence identity to the amino acid sequence of SEQ ID NO: 49.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising an HC-CDR1 comprising the amino acid sequence of SEQ ID NO: 2, an HC-CDR2 comprising the amino acid sequence of SEQ ID NO: 7, and an HC-CDR3 comprising the amino acid sequence of SEQ ID NO: 13, or a variant thereof comprising up to 5 amino acid substitutions in the HC-CDRs; and a V_{L} comprising an LC-CDR1 comprising the amino acid sequence of SEQ ID NO: 19, an LC-CDR2 comprising the amino acid sequence of SEQ ID NO: 25, and an LC-CDR3 comprising the amino acid sequence of SEQ ID NO: 30, or a variant thereof comprising up to 5 amino acid substitutions in the LC-CDRs. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of SEQ ID NO: 41 or a variant thereof having at least about 80% sequence identity to the amino acid sequence of SEQ ID NO: 41, and a V_{L} comprising the amino acid sequence of SEQ ID NO: 50, or a variant thereof having at least about 80% sequence identity to the amino acid sequence of SEQ ID NO: 50.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising an HC-CDR1 comprising the amino acid sequence of SEQ ID NO: 3, an HC-CDR2 comprising the amino acid sequence of SEQ ID NO: 8, and an HC-CDR3 comprising the amino acid sequence of SEQ ID NO: 14, or a variant thereof comprising up to 5 amino acid substitutions in the HC-CDRs; and a V_{L} comprising an LC-CDR1 comprising the amino acid sequence of SEQ ID NO: 20, an LC-CDR2 comprising the amino acid sequence of SEQ ID NO: 26, and an LC-CDR3 comprising the amino acid sequence of SEQ ID NO: 31, or a variant thereof comprising up to 5 amino acid substitutions in the LC-CDRs. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of SEQ ID NO: 42 or a variant thereof having at least about 80% sequence identity to the amino acid sequence of SEQ ID NO: 42, and a V_{L} comprising the amino acid sequence of SEQ ID NO: 51, or a variant thereof having at least about 80% sequence identity to the amino acid sequence of SEQ ID NO: 51.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15, or a variant thereof comprising up to 5 amino acid substitutions in the HC-CDRs; and a V_{L} comprising an LC-CDR1 comprising the amino acid sequence of SEQ ID NO: 21, an LC-CDR2 comprising the amino acid sequence of SEQ ID NO: 26, and an LC-CDR3 comprising the amino acid sequence of SEQ ID NO: 32, or a variant thereof comprising up to 5 amino acid substitutions in the LC-CDRs. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of SEQ ID NO: 43 or a variant thereof having at least about 80% sequence identity to the amino acid sequence of SEQ ID NO: 43, and a V_{L} comprising the amino acid sequence of SEQ ID NO: 52, or a variant thereof having at least about 80% sequence identity to the amino acid sequence of SEQ ID NO: 52.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 15, or a variant thereof comprising up to 5 amino acid substitutions in the HC-CDRs; and a V_{L} comprising an LC-CDR1 comprising the amino acid sequence of SEQ ID NO: 22, an LC-CDR2 comprising the amino acid sequence of SEQ ID NO: 27, and an LC-CDR3 comprising the amino acid sequence of SEQ ID NO: 33, or a variant thereof comprising up to 5 amino acid substitutions in the LC-CDRs. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of SEQ ID NO: 43 or a variant thereof having at least about 80% sequence identity to the amino acid sequence of SEQ ID NO: 43, and a V_{L} comprising the amino acid sequence of SEQ ID NO: 53, or a variant thereof having at least about 80% sequence identity to the amino acid sequence of SEQ ID NO: 53.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

For example, in some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising an HC-CDR1 comprising the amino acid sequence of SEQ ID NO: 5, an HC-CDR2 comprising the amino acid sequence of SEQ ID NO: 10, and an HC-CDR3 comprising the amino acid sequence of SEQ ID NO: 16, or a variant thereof comprising up to 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 28, and an LC-CDR3 comprising the amino acid sequence of SEQ ID NO: 34, or a variant thereof comprising up to 5 amino acid substitutions in the LC-CDRs. In some embodiments, the anti-PD-L1 antibody is a full-length antibody. In some embodiments, the full-length anti-PD-L1 antibody is an IgG1 or IgG4 antibody. In some embodiments, the disease or condition is selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus. In some embodiments, the individual is human.

In some embodiments, there is provided a method of treating an individual having a disease or condition associated with the activity and/or expression of PD-L1 or a T-cell dysfunction disorder (e.g., cancer or infectious disease) comprising administering to the individual an effective amount of a pharmaceutical composition comprising an anti-PD-L1 antibody (e.g., full-length anti-PD-L1 antibody) comprising a V_{H} comprising the amino acid sequence of SEQ ID NO: 44 or a variant thereof having at least about 80% sequence identity to the amino acid sequence of SEQ ID NO: 44, and a V_{L} comprising the amino acid sequence of SEQ ID NO: 54, or a variant thereof having at least about 80% sequence identity to the amino acid sequence of SEQ ID NO: 54.

In some embodiments, the anti-PD-L1 antibody provided herein is a full-length anti-PD-L1 antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 55. In some embodiments, the heavy chain constant region comprises or consists of the amino acid sequence of SEQ ID NO: 56. In some embodiments, the light chain constant region comprises or consists of the amino acid sequence of SEQ ID NO: 57. In some embodiments, the light chain constant region comprises or consists of the amino acid sequence of SEQ ID NO: 58.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as cancer or infectious disease). In some embodiments, the individual has one or more risk factors associated with one or more diseases or disorders described herein.

The present application in some embodiments provides a method of delivering an anti-PD-L1 antibody (such as any one of the anti-PD-L1 antibodies described herein, e.g., an isolated anti-PD-L1 antibody) to a cell producing PD-L1 in an individual, the method comprising administering to the individual a composition comprising the anti-PD-L1 antibody.

Many diagnostic methods for cancer or infectious disease or any other disease associated with the activity and/or expression of PD-L1 or a T-cell dysfunction disorder and the clinical delineation of those diseases are known in the art. Such methods include, but are not limited to, e.g., immunohistochemistry, PCR, and fluorescent in situ hybridization (FISH).

In some embodiments, the anti-PD-L1 antibodies (e.g., full-length anti-PD-L1 antibodies) and/or compositions of the application are administered in combination with a second, third, or fourth agent (including, e.g., radiation therapy, chemotherapy, targeted therapy, immunotherapy, hormonal therapy, angiogenesis inhibition and palliative care) to treat diseases or disorders associated with the activity and/or expression of PD-L1 or a T-cell dysfunction disorder.

In some embodiments, cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the efficiency of treatment is measured as the percentage tumor growth inhibition (% TGI), calculated using the equation 100−(T/C×100), where T is the mean relative tumor volume of the treated tumor, and C is the mean relative tumor volume of a non-treated tumor. In some embodiments, the % TGI is about 10%, about 20%, about 30%, about 40%, about 50%, about 60%, about 70%, about 80%, about 90%, about 91%, about 92%, about 93%, about 94%, about 95%, or more than 95%. In some embodiments, the efficacy of treatment is measured using shape change of granulocytes and/or increase in the survival of granulocytes. In some embodiments, the efficacy of treatment is measured by the increase of cytokine secretion by monocytes.

### Dosing and Method of Administering the Anti-PD-L1 Antibodies

The dose of the anti-PD-L1 antibody (such as isolated anti-PD-L1 antibody) compositions administered to an individual (such as a human) may vary with the particular composition, the mode of administration, and the type of disease being treated. In some embodiments, the amount of the composition (such as composition comprising isolated anti-PD-L1 antibody) is effective to result in an objective response (such as a partial response or a complete response) in the treatment of cancer or infectious disease. In some embodiments, the amount of the anti-PD-L1 antibody composition is sufficient to result in a complete response in the individual. In some embodiments, the amount of the anti-PD-L1 antibody composition is sufficient to result in a partial response in the individual. In some embodiments, the amount of the anti-PD-L1 antibody composition administered (for example when administered alone) is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-PD-L1 antibody composition. Responses of an individual to the treatment of the methods described herein can be determined, for example, based on tumor size, tumor burden, progression free survival (PFS), overall survival (OS).

In some embodiments, the amount of the composition (such as composition comprising isolated anti-PD-L1 antibody) is sufficient to control symptoms and reduce the risk of exacerbations of the individual. In some embodiments, the amount of the composition is sufficient to control symptoms and reduce the risk of exacerbations of the individual. In some embodiments, the amount of the composition (for example when administered along) is sufficient to produce clinical benefit of more than about any of 50%, 60%, 70%, or 77% among a population of individuals treated with the anti-PD-L1 antibody composition.

In some embodiments, the amount of the composition (such as composition comprising isolated anti-PD-L1 antibody), alone or in combination with a second, third, and/or fourth agent (such as radiation therapy, chemotherapy, targeted therapy, immunotherapy, hormonal therapy, angiogenesis inhibition and palliative care), is an amount sufficient to control symptoms and reduce the risk of exacerbations in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-PD-L1 antibody (such as a full-length anti-PD-L1 antibody) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regimen. In some embodiments, the amount of the composition is more than about any of 80%, 90%, 95%, or 98% of the MTD.

In some embodiments, the amount of an anti-PD-L1 antibody (such as a full-length anti-PD-L1 antibody) in the composition is included in a range of about 0.001 μg to about 1000 μg.

In some embodiments of any of the above aspects, the effective amount of anti-PD-L1 antibody (such as a full-length anti-PD-L1 antibody) in the composition is in the range of about 0.1 μg/kg to about 100 mg/kg of total body weight.

The anti-PD-L1 antibody compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal or transdermal. In some embodiments, sustained continuous release formulation of the composition may be used. In some embodiments, the composition is administered inhaled. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically. In some embodiments, the composition is administered by hepatic arterial infusion. In some embodiments, the administration is to an injection site distal to a first disease site.

### Articles of Manufacture and Kits

In some embodiments of the application, there is provided an article of manufacture containing materials useful for the treatment of disease or condition associated with the activity and/or expression of PD-L1 or a T-cell dysfunction disorder, (e.g., cancer or infectious disease) or for delivering an anti-PD-L1 antibody (such as a full-length anti-PD-L1 antibody) to a cell producing PD-L1 of the individual. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-PD-L1 antibody of the application. The label or package insert indicates that the composition is used for treating the particular condition. The label or package insert will further comprise instructions for administering the anti-PD-L1 antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating disease or condition associated with the activity and/or expression of PD-L1 or a T-cell dysfunction disorder (such as cancer or infectious disease). In some embodiments, the package insert indicates that the composition is used for treating disease or condition selected from the group consisting of melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus.

Additionally, the article of manufacture may further comprise a second container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

Kits are also provided that are useful for various purposes, e.g., for treatment of disease or condition associated with the activity and/or expression of PD-L1 or a T-cell dysfunction disorder (e.g., cancer or infectious disease), or for delivering an anti-PD-L1 antibody (such as a full-length anti-PD-L1 antibody) to a cell producing PD-L1 of the individual, optionally in combination with the articles of manufacture. Kits of the application include one or more containers comprising anti-PD-L1 antibody composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the application are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-PD-L1 antibody (such as a full-length anti-PD-L1 antibody). In some embodiments, the kit comprises a) a composition comprising any one of the anti-PD-L1 antibodies described herein, and b) an effective amount of at least one other agent, wherein the other agent enhances the effects (e.g., treatment effect, detecting effect) of the anti-PD-L1 antibody. In some embodiments, the kit comprises a) a composition comprising any one of the anti-PD-L1 antibodies described herein, and b) instructions for administering the anti-PD-L1 antibody composition to an individual for treatment of a disease or condition associated with the activity and/or expression of PD-L1 or a T-cell dysfunction disorder (e.g., cancer or infectious disease). In some embodiments, the kit comprises a) a composition comprising any one of the anti-PD-L1 antibodies described herein, b) an effective amount of at least one other agent, wherein the other agent enhances the effect (e.g., treatment effect, detecting effect) of the anti-PD-L1 antibody, and c) instructions for administering the anti-PD-L1 antibody composition and the other agent(s) to an individual for treatment of a disease or condition associated with the activity and/or expression of PD-L1 or a T-cell dysfunction disorder (e.g., cancer or infectious disease. The anti-PD-L1 antibody and the other agent(s) can be present in separate containers or in a single container. For example, the kit may comprise one distinct composition or two or more compositions wherein one composition comprises an anti-PD-L1 antibody and another composition comprises another agent.

In some embodiments, the kit comprises a nucleic acid (or a set of nucleic acids) encoding an anti-PD-L1 antibody (such as a full-length anti-PD-L1 antibody). In some embodiments, the kit comprises a) a nucleic acid (or a set of nucleic acids) encoding an anti-PD-L1 antibody, and b) a host cell for expressing the nucleic acid (or a set of nucleic acids). In some embodiments, the kit comprises a) a nucleic acid (or a set of nucleic acids) encoding an anti-PD-L1 antibody, and b) instructions for i) expressing the anti-PD-L1 antibody in a host cell, ii) preparing a composition comprising the anti-PD-L1 antibody, and iii) administering the composition comprising the anti-PD-L1 antibody to an individual for the treatment of a disease or condition associated with the activity and/or expression of PD-L1 or a T-cell dysfunction disorder (e.g., cancer or infectious disease. In some embodiments, the kit comprises a) a nucleic acid (or a set of nucleic acids) encoding an anti-PD-L1 antibody, b) a host cell for expressing the nucleic acid (or a set of nucleic acids), and c) instructions for i) expressing the anti-PD-L1 antibody in the host cell, ii) preparing a composition comprising the anti-PD-L1 antibody, and iii) administering the composition comprising the anti-PD-L1 antibody to an individual for the treatment of a disease or condition associated with the activity and/or expression of PD-L1 or a T-cell dysfunction disorder (e.g., cancer or infectious disease).

The kits of the application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-PD-L1 antibody compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-PD-L1 antibody (such as a full-length anti-PD-L1 antibody) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-PD-L1 antibody and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this application. The application will now be described in greater detail by reference to the following non-limiting examples. The following examples further illustrate the application but, of course, should not be construed as in any way limiting its scope.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Generation of PD-L1 Polypeptides

This example illustrates the preparation of the various PD-L1 polypeptide constructs used as antigens in eliciting and screening the anti-PD-L1 antibodies of the present disclosure.

Generation of recombinant PD-L1-Fc fusion proteins: The coding sequence of the extracellular domain (ECD) of human PD-L1 gene, the ECD of mouse PD-L1 gene, or a chimeric of human PD-L1 V domain and mouse PD-L1 C domain genes were synthesized (IDT DNA company) and sub-cloned into the expression vectors pTTa1 using restriction enzyme with recognition sites. The amino acid sequences were provided in Table 5. All constructs had the following C-terminal mouse IgG2a Fc (mIgG2aFc) for purification and detection purposes.

TABLE 5
PD-L1 polypeptide sequence

| SEQ ID | | |
|---|---|---|
| NO | Protein | sequence |
| 59 | human PD-L1 ECD | FTVTVPKDLYVVEYGSNMTIECKFPVEKQLDLAALIVYWEM |
| | | EDKNIIQFVHGEEDLKVQHSSYRQRARLLKDQLSLGNAALQI |
| | | TDVKLQDAGVYRCMISYGGADYKRITVKVNAPYNKINQRIL |
| | | VVDPVTSEHELTCQAEGYPKAEVIWTSSDHQVLSGKTTTTNS |
| | | KREEKLFNVTSTLRINTTTNEIFYCTFRRLDPEENHTAELVIPE |
| | | LPLAHPPNER |
| 60 | Mouse PD-L1 ECD | FTITAPKDLYVVEYGSNVTMECRFPVERELDLLALVVYWEKE |
| | | DEQVIQFVAGEEDLKPQHSNFRGRASLPKDQLLKGNAALQIT |
| | | DVKLQDAGVYCCIISYGGADYKRITLKVNAPYRKINQRISVDP |
| | | ATSEHELICQAEGYPEAEVIWTNSDHQPVSGKRSVTTSRTEG |
| | | MLLNVTSSLRVNATANDVFYCTFWRSQPGQNHTAELIIPELP |
| | | ATHPPQNRTH |
| 61 | human PD-L1 ECD | FTVTVPKDLYVVEYGSNMTIECKFPVEKQLDLAALIVYWEM |
| | mIgG2aFc | EDKNIIQFVHGEEDLKVQHSSYRQRARLLKDQLSLGNAALQI |
| | | TDVKLQDAGVYRCMISYGGADYKRITVKVNAPYNKINQRIL |
| | | VVDPVTSEHELTCQAEGYPKAEVIWTSSDHQVLSGKTTTTNS |
| | | KREEKLFNVTSTLRINTTTNEIFYCTFRRLDPEENHTAELVIPE |
| | | LPLAHPPNERTEPRGPTIKPCPPCKCPAPNLLGGPSVFIFPPKIK |
| | | DVLMISLSPIVTCVVVDVSEDDPDVQISWFVNNVEVHTAQTQ |
| | | THREDYNSTLRVVSALPIQHQDWMSGKEFKCKVNNKDLPAPI |
| | | ERTISKPKGSVRAPQVYVLPPPEEEMTKKQVTLTCMVTDFMP |
| | | EDIYVEWTNNGKTELNYKNTEPVLDSDGSYFMYSKLRVEKK |
| | | NWVERNSYSCSVVHEGLHNHHTTKSFSRTPGK |
| 62 | mouse PD-L1 ECD | FTITAPKDLYVVEYGSNVTMECRFPVERELDLLALVVYWEKE |
| | mIgG2aFc | DEQVIQFVAGEEDLKPQHSNFRGRASLPKDQLLKGNAALQIT |
| | | DVKLQDAGVYCCIISYGGADYKRITLKVNAPYRKINQRISVDP |
| | | ATSEHELICQAEGYPEAEVIWTNSDHQPVSGKRSVTTSRTEG |
| | | MLLNVTSSLRVNATANDVFYCTFWRSQPGQNHTAELIIPELP |
| | | ATHPPQNRTHGSEPRGPTIKPCPPCKCPAPNLLGGPSVFIFPPKI |
| | | KDVLMISLSPIVTCVVVDVSEDDPDVQISWFVNNVEVHTAQT |
| | | QTHREDYNSTLRVVSALPIQHQDWMSGKEFKCKVNNKDLPA |
| | | PIERTISKPKGSVRAPQVYVLPPPEEEMTKKQVTLTCMVTDF |
| | | MPEDIYVEWTNNGKTELNYKNTEPVLDSDGSYFMYSKLR |
| | | VEKKNWVERNSYSCSVVHEGLHNHHTTKSFSRTPGK |
| 63 | human and | FTVTVPKDLYVVEYGSNMTIECKFPVEKQLDLAALIVYWEM |
| | mouse chimeric | EDKNIIQFVHGEEDLKVQHSSYRQRARLLKDQLSLGNAALQI |
| | ECD PD- | TDVKLQDAGVYRCMISYGGADYKRITVKVNAPYRKINQRISV |
| | L1 mIgG2a Fc | DPATSEHELICQAEGYPEAEVIWTNSDHQPVSGKRSVTTSRTE |
| | | GMLLNVTSSLRVNATANDVFYCTFWRSQPGQNHTAELIIPEL |
| | | PATHPPQNRTHEPRGPTIKPCPPCKCPAPNLLGGPSVFIFPPKIK |
| | | DVLMISLSPIVTCVVVDVSEDDPDVQISWFVNNVEVHTAQTQ |
| | | THREDYNSTLRVVSALPIQHQDWMSGKEFKCKVNNKDLPAPI |
| | | ERTISKPKGSVRAPQVYVLPPPEEEMTKKQVTLTCMVTDFMP |
| | | EDIYVEWTNNGKTELNYKNTEPVLDSDGSYFMYSKLRVEKK |
| | | NWVERNSYSCSVVHEGLHNHHTTKSFSRTPGK |

Expression of these PD-L1 ECD mIgG2aFc fusion proteins were carried out according to manufacturer's protocol. Briefly, Expi293 cells were transfected with the expression vectors, and the cells were cultured at 37° C., under 800 CO₂ and 120 rpm for 5 days.

For purification of Fc-fusion proteins, the culture media were harvested and the PD-L11 mIgG2aFc fusion proteins were purified using a protein A column (5 mL Hitrap, Cytiva) using the protocol provided by the manufacturer's guideline. Briefly, a Protein A column was first equilibrated with 1×PBS buffer (pH7.4, Gibco). The supernatant of the culture media was filtered and passed through the column. The loaded column was then washed using 1×PBS buffer. The protein was eluted using 50 mM sodium citrate (pH 3.0) and the elution containing PD-L1 ECD mIgG2aFc was collected.

### Example 2: Generation of Anti-Human PD-L1 Antibodies Using Hybridoma Methods, Screening and Characterization

This example illustrates the methods of using mouse hybridoma technology to generate anti-human PD-L1 antibodies, and methods to screen and select antibodies for further characterization.

Immunizations and fusions: Balb/c, C57BL/6 and NZB mice were immunized with recombinant extracellular domains of human PD-L1 fused with mouse IgG2a Fc, or the V-domain of human PD-L1 fused with the C-domain of mouse PD-L1 and mouse IgG2a Fc, adjuvanted with RIBI (Sigma Aldrich—St Louis, MO, USA; cat #56322-1VL), Titermax (Sigma Aldrich—St Louis, MO, USA; cat #T2684-1ML), Freund's (Freund's adjuvant, incomplete (Sigma Aldrich—St Louis, MO, USA; cat #F5506-10x-10 ml) or in alternative order. Endpoint titers were determined by ELISA as described below. Three days after the last immunization, spleens and lymph nodes were harvested, and processed according to standard protocols. Mouse B cells were isolated by EasySep Mouse B cell isolation Kit (StemCell, Cambridge, MA, USA; cat #19854A) and fused with myeloma cells SP2/0-Ag14 cells (American Type Culture Collection CRL 1581) using PEG. Following standard protocols, the fused cells were plated into six-well plates in semi-solid ClonalCell-HY Cloning-Medium D (StemCell, Cambridge, MA, USA; cat #03804). Monoclonal Hybridoma clones were picked into 96 well/plate using Clone Pix 2 Machine (Molecular Devices, San Jose, CA, USA) and cultured in low-Ig HT medium.

ELISA screening assay: After 10-14 days of culture, supernatants were collected and subjected to primary screening by ELISA with 96 well ELISA plates coated with human PD-L1 extracellular domain proteins with human IgG Fc tag (Sinobiological, Cat: 10084-H02H). 96-well round bottom ELISA plates (corning 3366, VWR, Visalia, CA, USA; cat #25381-051) were coated overnight at 4° C. with 50 μL/well of the PD-L1-human Fc protein at a concentration of 1 μg/mL in coating buffer (1× phosphate buffered saline, PBS). After washing five times with 300 μL of PBS, 0.05% TWEEN®-20 (wash buffer), the plates were blocked by addition of 250 μL/well of assay/blocking solution containing 1% bovine serum albumin (BSA) in phosphate buffered saline (PBS) (pH 7.4), and incubated at room temperature for 1 hour. Plates were then washed 5 times with wash buffer. 50 μL of culture supernatant of individual hybridoma clones was added to individual wells followed by incubation at 37° C. for 1 hour. Plates were washed, and 50 μL/well of goat anti-mouse antibody-conjugated with AP (Southern Biotech, Birmingham, AL, USA; cat #1030-04) was added at 1:2000 dilution in ELISA diluent. The plates were incubated at room temperature for 1 hour, washed 5 times with wash buffer and developed for 30 minutes by addition of 50 μL/well of Sigma Fast p-Nitrophenyl phosphate tablet (Sigma Aldrich—St Louis, MO, USA; cat #N2770-50SET). Plates were analyzed with Synergy HT (Bio-TEK, Vermont, USA) at 405 nm.

The parental hybridoma hits identified from the primary screen were expanded to 48 or 24-well plates and a confirmatory ELISA was performed following the primary screen protocol, to further confirm and screen for anti-human PD-L1 binders.

Inhibition of PD-1 and PD-L1 binding assay of hybridoma hits: The supernatants of the hybridoma hits identified in the primary screen were further tested for their ability to inhibit the interaction between PD-L1 and PD1 proteins. The HTRF (Homogeneous Time-resolved Fluorescence) technology was chosen to enable high-throughput characterization of antibody blockers by using the protocol modified from Cisbio company (www.cisbio.com). Briefly, in 384 well white-plate (Corning 4512), the Europium-conjugated human PD-L1 was pre-mixed with Alxa-647-conjugated human PD-1 with the ratio of 1:10 in molar concentrations. The supernatants of the hybridoma to be screened were then added into plate in each well. When the donor and acceptor dyes are brought into close proximity due to PD-L1 and PD1 binding, excitation of the donor protein (Europium-human PD-L1) triggers fluorescence resonance energy transfer (FRET) towards the acceptor protein (human PD-1-Alexa-647), which in turn emits specifically at 665 nm. The specific signal is directly proportional to the extent of PD1/PD-L1 interaction. Thus, antibody blocking PD1/PD-L1 interaction will cause a reduction in HTRF signal which can be read out in Biotek Synergy Neo2 reader. For each individual well the ratio between the acceptor and donor emission signals is calculated as below: Ratio=(Signal 665 nm/Signal 620 nm)×10⁴.

For the hybridoma screening, 10 μL of the supernatants from hybridoma cell culture were added into the 384 well white-plate in duplicate wells containing 10 μL of the pre-mixed PD-1/PD-L1 proteins (in-house made and conjugated). After 2 hours in room temperature, the plates were read for the signals in Biotek Synergy Neo2 reader. By comparing with the reference antibody Atezolizumab (anti-PD-L1 antibody, Roche; referred to as “Ref” in the following), the hybridoma clones from which the antibodies showed more than 40% of inhibition of the PD-1/PD-L1 interaction were chosen for immunoglobulin gene sequencing and antibody expression in Expi-293 cells.

### Sequencing and Amplification of Hybridoma Antibody Clones

RNA Extraction: Monoclonal anti-human PD-L1 hybridoma hits were grown to a density of 1-3×10⁵ in standard hybridoma medium (DMEM/F12, 10% FBS, 1% Glutamax, 1% pen/strep) for 7-10 days in a T75 flask with >80% cell viability. 1-3 million cells from cultures were pelleted in a 15 mL falcon tube after centrifugation at 300 g for 5 min. Pelleted cells were washed by 5 mL ice cold PBS. PBS was removed and cells were resuspended in 600 μL Buffer RLT Plus (Qiagen, cat #74134). Total RNA was isolated from the lysate following the manufacturing protocol (Qiagen, cat #74134).

PCR amplification to generate cDNA. The synthesis of cDNA utilizes specific reverse PCR primers in conjunction with switch oligos for heavy and kappa chains. To generate cDNA, one microgram of RNA was used as a template followed by reverse transcription using SMART Scribe Reverse Transcriptase kit from Clontech (TAKARA, cat #639537). Additionally, the reagents include 10 μM primers (Integrated DNA technologies), 10 mM deoxy nucleotide triphosphate mix (New England Biolab, cat #N0447S), H₂O, and an 80 U/μL RNAse inhibitor (Invitrogen, cat #10000840). The constant region-specific reverse primers were used in conjunction with universal forward primer in 5′-RACE PCR reactions. PCR products were gel purified and cloned into TOPO TA vectors (ThermoFisher, cat #451641) which were then transformed into competent cells (ThermoFisher, cat #451641). After transformation and blue/white screening, white colonies were picked and grew overnight in LB broth media containing carbenicillin. Miniprep purified plasmids were sequenced using M13 forward and T7-forward primers.

### Example 3: In Vitro Assays of Anti-Human PD-L1 Chimeric Antibodies

This example illustrates cell-based assays used to characterize the functional activity of the anti-human PD-L1 chimeric antibodies.

### Generation of Recombinant IgG1 Versions of Anti-Human PD-L1 Antibodies

The recombinant chimeric anti-human PD-L1 antibody constructs with heavy and light chain variable domain of mouse anti-human PD-L1 antibodies and human constant regions were made by using methods well known in the field. Briefly, the variable regions of heavy and light chain (V_{H} and V_{L}) sequences of antibody candidates were PCR amplified using the template plasmids cloned from the hybridomas. The V_{H} and V_{L} domains were then subcloned into the expression vectors pTTa1 containing constant regions of human IgG1 heavy chain and Kappa genes using restriction enzyme with recognition sites. The exemplary human heavy chain constant region and light chain constant region were shown in Table 3. The expression of recombinant anti-human PD-L1 chimeric antibodies was performed using Expi293 expression system in accordance with the instruction provided. The plasmids for the heavy chain and the light chain were co-transfected into the cells at the ratio of 1:1, and the transfected cells were cultured at 37° C., under 8% CO₂ and 120 rpm for 5 days before harvest.

Recombinant IgG molecules were purified as the following protocols. The culture media were collected and the chimeric anti-PD-L1 antibodies were purified using a Protein A column (5 mL Hitrap, Cytiva) using the protocol provided by manufacturer's guidelines. Briefly, a Protein A column was first equilibrated with 1×PBS buffer (pH7.4, Gibco). The supernatant of the culture media was filtered and passed through the column. The loaded column was then washed with 1×PBS buffer. The anti-PD-L1 antibody was eluted using 50 mM sodium citrate (pH 3.0) and the elution containing the chimeric antibody was collected. The variable domain sequences of the chimeric anti-human PD-L1 antibody candidates are summarized in Table 2 and 3 and provided in the attached Sequence Listing.

### Inhibition of PD-1 and PD-L1 Binding Assay

Inhibition of PD-1 and PD-L1 binding assay was performed on the purified recombinant chimeric antibodies. Briefly, the recombinant antibodies were diluted serially at varying concentrations and the inhibition activities on PD-1 and PD-L1 interaction were detected by the HTRF methods described above in Example 2.

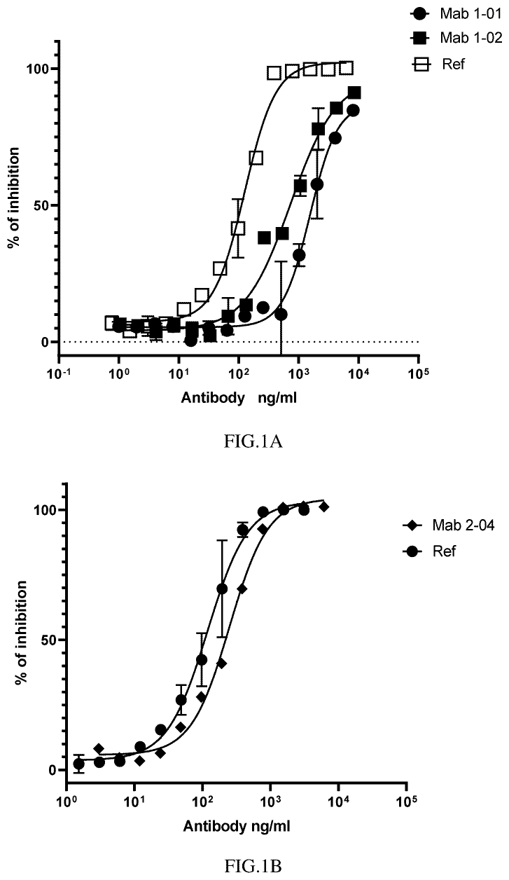

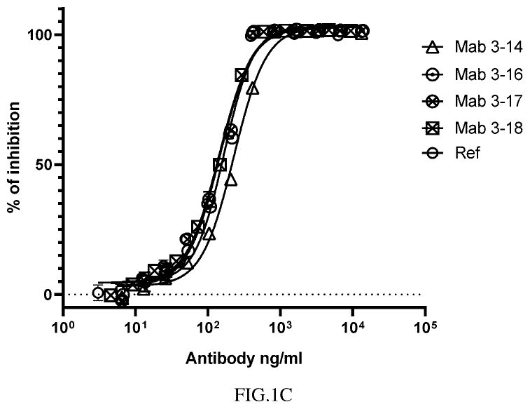

As depicted in FIGS. 1A to 1C, the chimeric anti-human PD-L1 antibody Mab 1-01, Mab 1-02, Mab 2-04, Mab 3-14, Mab 3-16, Mab 3-17 and Mab 3-18 show the dose-dependent inhibition of the PD-1 and PD-L1 interaction.

### Reporter Cell Assay

The PD-1/PD-L1 signaling pathway inhibits TCR/CD28 co-stimulatory signals, which can be read out as reduced cytokine production, such as IL-2 and IFN-γ. Accordingly, inhibition of the PD-1 pathway via blocking PD-1 interaction with PD-L1 by the antibody to PD-L1 can enhance T cell activation. In reporter cell assay, Jurkat 6E-1 cell line (ATCC) is derived from human acute T lymphocyte leukemia. This cell is characterized by production of large amount of IL-2 after stimulation, such as with phorbol esters. Jurkat 6E-1 cell is engineered with human PD-1 gene. Raji cell line, a human B lymphocyte-derived cell, expressing human PD-L1 (www.Invivogen.com) was used as antigen presenting cells. The ability of anti PD-L1 antibodies to enhance IL-2 production can be observed after presenting T cells with super antigen such as Staphylococcal enterotoxin E (SEE) by antigen presenting cells (APCs).

The human PD-1 gene engineered Jurkat cell line was made in-house by using lentivirus mediated gene integration method. Briefly, the human PD-1 gene (NP_005009.2) was synthesized (IDT DNA company) and subcloned into the lentivirus vector. The infectious virus particles were packaged by using lentivirus packaging system (Clontech, Lenti-X™ Packaging Single Shots, VSV-G). After infection, the Jurkat cell was subjected to selection by the antibiotic Blastcidin (3 μg/ml) for 3-4 weeks until the Jurkat cells were established to be almost 100% positive to PD-1 by using FACS analysis with anti-human PD-1 antibody (APC-anti-human PD-1, Biolegend). The human PD-L1-expressing Raji Cell line was purchased from www.Invivogen.com.

The Jurkat and Raji cells were maintained in RPMI 1640 containing 10% FBS, 1% L Glutamine and 0.1% Pen/Strep in 5% CO₂ at 37° C. separately. In the reporter cell assay, the Raji B cells in 1×10⁶/ml were pre-incubated with 2-3 ng/ml SEE superantigen (Staphylococcal enterotoxin E, Toxin Technology #ET404) and then mixed with equal volume of the Jurkat cells in 2×10⁶/ml. 100 μl of the Jurkat and Raji cell mixtures and 100 μl of the antibodies with 1:3 serially diluted concentration were transferred into a 96-well U-bottom plate in triplicate wells containing RPMI 1640, 10% FBS, 1% L Glutamine and 0.1% Pen/Strep in 5% CO₂ at 37° C. The culture supernatants were collected after 48 hours. Human IL-2 was quantified by ELISA using IL-2 ELISA kit (Biolegend MAX Human IL-2 ELISA kit) according to its protocol.

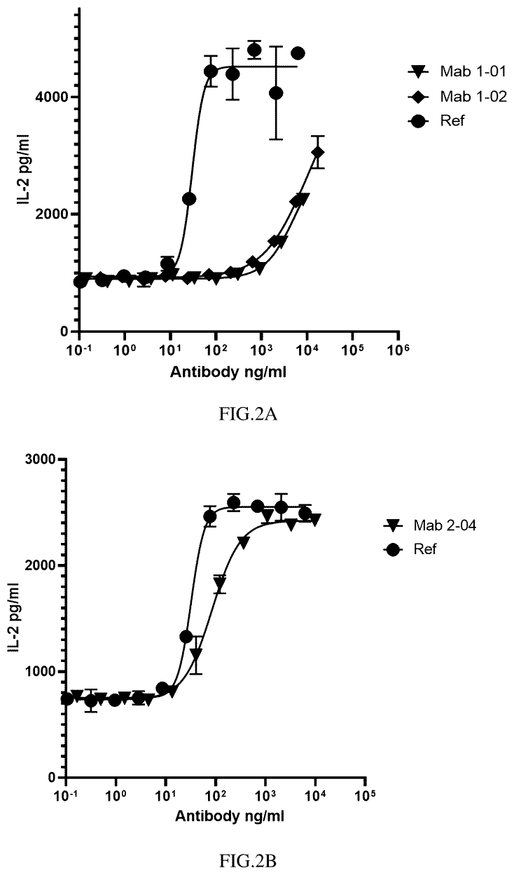

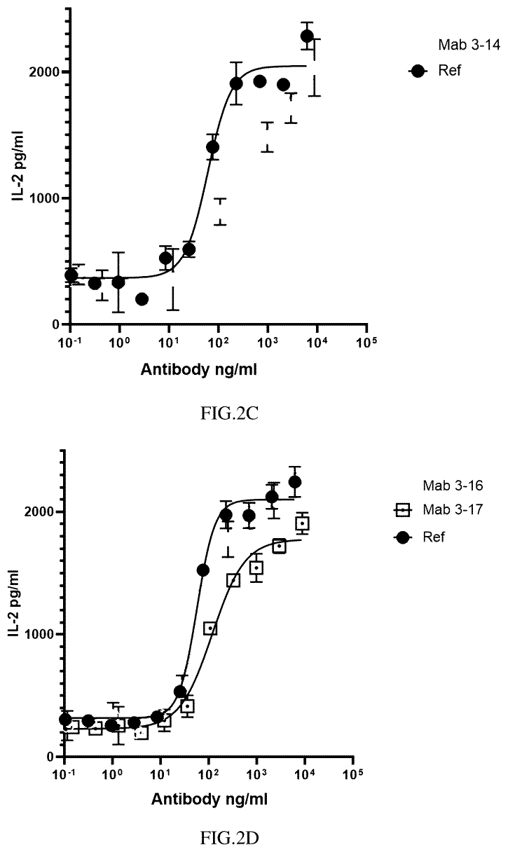

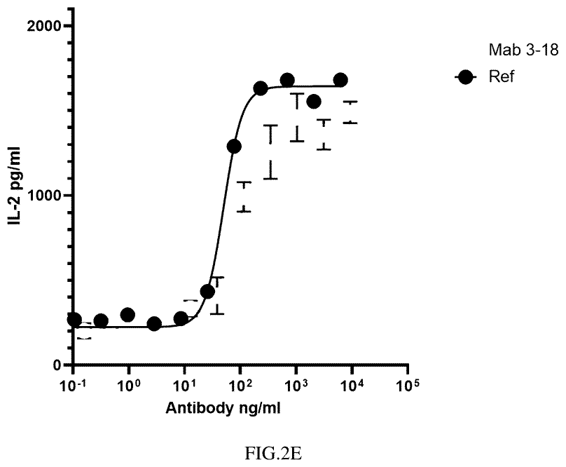

As shown in FIGS. 2A to 2E, the anti-human PD-L1 chimeric antibodies Mab 1-01, Mab 1-02, Mab 2-04, Mab 3-14, Mab 3-16, Mab 3-17 and Mab 3-18 exhibited a dose-dependent response in the level of IL-2 secretion after co-stimulation. The Mab 3-16 had shown the best efficacy in this functionality.

### Characterization of Binding Affinity and Dissociation Constant (Kd) of the Chimeric Anti-Human PD-L1 Antibodies

Binding affinities of anti-human PD-L1 antibodies were determined using ForteBio BioLayer Interferometry (BLI) technology with the Octet Red system. In the affinity assay, the chimeric anti-human PD-L1 antibody with human IgG1 isotype was captured by the anti-human Fc sensors (AHC, Fortebio) at the loading concentration of 2 μg/mL in kinetic buffer for 300 seconds. For each assay, an array of human PD-L1 (Sino biological, 10084-H08H) with the concentrations from about 50 nM to less than 1 nM in the kinetic buffer were tested by the antibody-captured sensors with the association time of 300 seconds and dissociation time of 1200 seconds. The kinetic data were analysis using program Data Analysis HT 10.0 according to the manual. All the data were repeated in at least two independent assays.

Table 6 shows the affinity parameter of the anti-human PD-L1 antibodies and the reference antibody.

TABLE 6

| Antibody | Kd(M) | Kon (1/Ms) | Koff(1/s) |
|---|---|---|---|
| Mab 1-01 | 1.30E−08 | n.d. | n.d. |
| Mab 1-02 | 1.60E−08 | n.d. | n.d. |
| Mab 2-04 | 2.79E−10 | 8.64E+05 | 2.41E−04 |
| Mab 3-14 | 1.24E−10 | 3.27E+05 | 4.05E−05 |
| Mab 3-16 | 1.46E−10 | 4.23E+05 | 6.17E−05 |
| Mab 3-17 | 1.50E−09 | 4.17E+05 | 6.26E−04 |
| Mab 3-18 | 1.16E−09 | 3.51E+05 | 4.08E−04 |
| Ref | 5.65E−10 | 2.20E+05 | 1.25E−04 |

### Effect of Anti-Human PD-L1 Antibodies on IL-2 Secretion by Staphylococcal Enterotoxin B Stimulated PBMCs.

The PD-1/PD-L1 signaling pathway inhibits TCR/CD28 co-stimulatory signals, which can be read out as reduced cytokine production, such as IL-2 and IFN-γ. Accordingly, inhibition of the PD-1 pathway via blocking PD-1 interaction with PD-L1 by the antibody to PD-L1 can enhance T cell activation. In human PBMC assay, the ability of anti-human PD-L1 antibody to enhance IL-2 production can be observed after presenting T cells with super antigen such as Staphylococcal enterotoxin B (SEB) by antigen presenting cells (APCs). The mechanism is referred to crosslinking the T cell receptor (TCR) and MHC class II to activate CD4+ T cells via TCR/CD28 pathway.

PBMCs from healthy human donors were added into 96-well U bottom plates with 1×10⁵/well. The Mab 3-16, reference antibody or IgG1 control (Biolegend, catalog #403501) at serially diluted concentration were then added into the plate in triplicate wells with the staphylococcal enterotoxin B (SEB; Toxin Technology) at the concentration of 100 ng/ml in final. The total volume per well is 200 μl and the cells were cultured for 3 days in RPMI 1640 containing 10% FBS, 1% L Glutamine and 0.1% Pen/Strep in 5% CO₂ at 37° C. IL-2 levels in culture supernatants were measured by ELISA analysis (Biolegend MAX human IL-2 ELISA).

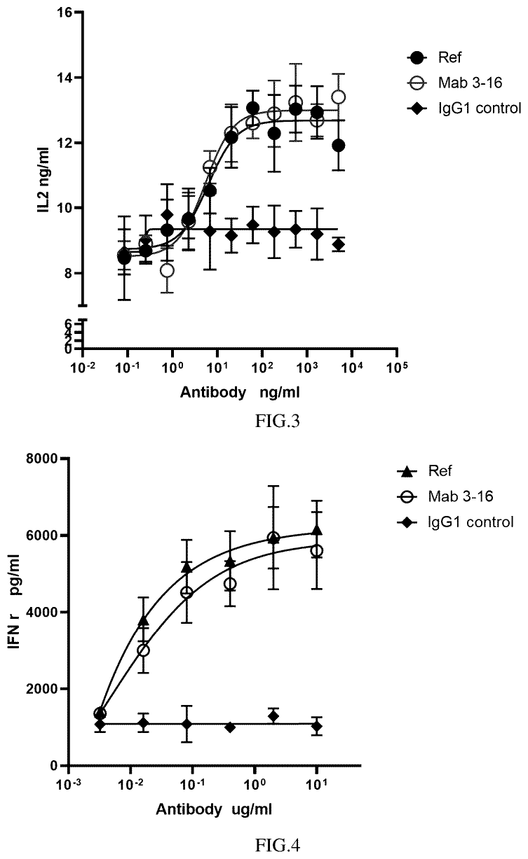

As shown in Table 7 and FIG. 3, Mab 3-16 efficiently and dose-dependently enhanced T cell activities represented as IL-2 production level. The EC₅₀ value of Mab 3-16 is comparable with the reference antibody.

TABLE 7

| Antibody | Mab 3-16 | Ref |
|---|---|---|
| EC₅₀(nM) | 5.413 | 6.251 |

### Effect of Anti-Human PD-L1 Antibodies on IFN-γ Production in a Mixed Lymphocyte Reaction (MLR)

The mixed lymphocyte reaction (MLR) is a classic culture method used for studying the allogeneic immune response in vitro, stemming from T cell alloreactivity. Depending on the host, approximately 1-10% of one's T cells recognize and respond to nonself antigen-MHC complexes; thus, for this assay there is no need for priming. Although it is unclear how a T cell receptor (TCR) recognize a peptide antigen/allo-MHC complex with high specificity, this assay creates a method to evaluate the effect of immune checkpoint inhibitors for further enhancement of the MLR response.

In MLR assay, human CD14+ monocytes were isolated from the PBMC of a donor using human monocyte negative isolation kit (Miltenyi biotec). The dendritic cells were derived from the purified CD14+ monocytes by using the dendritic cell generation media (PromoCell, C28050) according to the provided protocol. The allogeneic CD4+ T cells were prepared from the PBMC of a different donor using human CD4+ negative isolation kit (Miltenyi biotec). The dendritic cells and allogeneic CD4+ T cells were then mixed into 96 well U-bottom plate with 5×10⁵/ml of CD4+ T cell and 5×10⁴/ml of the dendritic cells in 100p complete AIM-V medium per well. 100p of the antibody Mab 3-16, reference antibody or IgG1 control (Biolegend, catalog #403501) at serially diluted concentrations were then transferred to the triplicate wells of the plate. After incubation for 72 hrs at 37° C. in 5% CO₂, the supernatants were harvested for the IFN-γ measurement by using the ELISA kit (Biolegend MAX human IFN-γ ELISA kit).

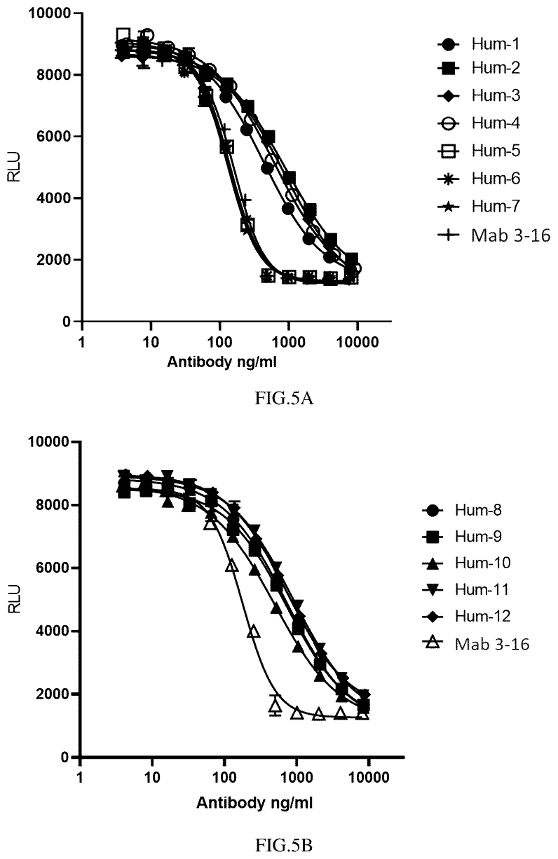

As shown in FIG. 4, Mab 3-16 promotes the IFN-γ secretion in dose-dependent manner, with the efficacy comparable to the reference antibody.

### Example 4: Preparation of Humanized Versions of Anti-Human PD-L1 Antibodies

This example illustrates the preparation of humanized versions of the murine anti-human PD-L1 antibody derived from the hybridoma clone Mab 3-16.

### Humanization of Murine Anti-Human PD-L1 Antibody

The anti-PD-L1 antibody Mab 3-16 from hybridoma was chosen for humanization based on its outstanding functional characteristics. The human heavy chain and light chain frameworks were obtained through blasting our in-house proprietary antibody humanization program. According to the best searching results, the human germline sequence of IGHV3-48*01 and the human germline of IGKV3-11*01 were chosen as recipients for the heavy chain and light chain of the antibody Mab 3-16 respectively.

Three CDR loops from the light and heavy chain of the antibody Mab 3-16 were grafted into the homologous human scaffold according to Kabat numbering method (Kabat et al., 1987). The amino acid residues which potentially affect the structure in the veneer zones were predicted by the program above. As the result, there are several back-mutations in the antibody light chain and in the heavy chain. These key residues from the prediction were back-mutated to corresponding positions individually or in combination, which, by pairing, give rise to 12 humanized antibodies, named as Hum-1 to Hum-12. These sequences were synthesized (IDT DNA company) and sub-cloned into antibody expression vectors of heavy and light chain separately for the recombinant antibody production.

The variable domain sequences of the humanized antibodies were summarized in Table 2 and 3.

### Example 5: In Vitro Assays of Humanized Anti-Human PD-L1 Antibodies Generation of Recombinant IgG Versions of Humanized Anti-Human PD-L1 Antibodies

The full-length IgG versions of humanized PD-L1 antibodies (reformatted as IgG1) were generated as described above in Example 3.

### PD-L1 Competitive Binding Assay

PD-L1 competitive binding assay is designed to rank the antibody affinity of humanization by their competitive capability to PD-L1 binding with their parent counterpart Mab 3-16. The HTRF (Homogeneous Time-resolved Fluorescence) technology was chosen to develop the assay by using the modified protocol described above. In this assay, the parent antibody Mab 3-16 was conjugated with the fluorescent dye of Alexa-647. Briefly in 384 well white-plate (Corning 4512), the Europium-conjugated human PD-L1 was pre-mixed with Alxa-647-conjugated antibody Mab 3-16 with the ratio of 1:10 in molar concentrations. The humanized antibodies were then added into the plate at serially diluted concentrations in duplicate wells. HTRF signal was then read out after 2 hours in room temperature in Biotek Synergy Neo2 reader.

As shown in FIGS. 5A and 5B, the Hum-5, Hum-6 and Hum-7 exhibited similar affinity when comparing to their parent antibody Mab 3-16.

### Characterization of Binding Affinity and Dissociation Constant (Kd) of the Humanized Antibodies

The binding affinity of anti-human PD-L1 antibodies were determined using ForteBio BioLayer Interferometry (BLI) as described above in Example 3. The affinities of the Hum-5, Hum-6 and Hum-7 were determined against both human PD-L1 and cynomolgus PD-L1 (Sino biological, 90251-C08H) proteins.

As shown in Table 8, the humanized antibody Hum-6 and Hum-7 share the almost identical affinity to their parent one (Mab 3-16). In addition, the affinities of the antibodies to cynomolgus PD-L1 and the human PD-L1 are very comparable.

TABLE 8

| | Human PD-L1 | | | Cynomolgus PD-L1 | | |
|---|---|---|---|---|---|---|
| Antibody | Kd(M) | Kon (1/Ms) | Koff (1/s) | Kd (M) | Kon (1/Ms) | Koff (1/s) |
| Hum-5 | 1.26E−10 | 3.10E+05 | 3.92E−05 | 9.15E−10 | 9.45E+04 | 8.65E−05 |
| Hum-6 | <1.0E−12 | 2.92E+05 | <1.0E−07 | 2.08E−12 | 6.39E+04 | 1.33E−07 |
| Hum-7 | <1.0E−12 | 3.02E+05 | 1.28E−07 | 1.10E−12 | 7.31E+04 | <1.0E−07 |
| Mab3-16 | <1.0E−12 | 2.86E+05 | 1.19E−07 | 8.63E−10 | 9.67E+04 | 8.34E−05 |

### Reporter Cell Assay

The antibody Hum-5, Hum-6 and Hum-7 were subjected to SEE reporter assay using the method described above in Example 3 to evaluate their functionality in triggering the IL-2 secretion of the Jurkat T cell line.

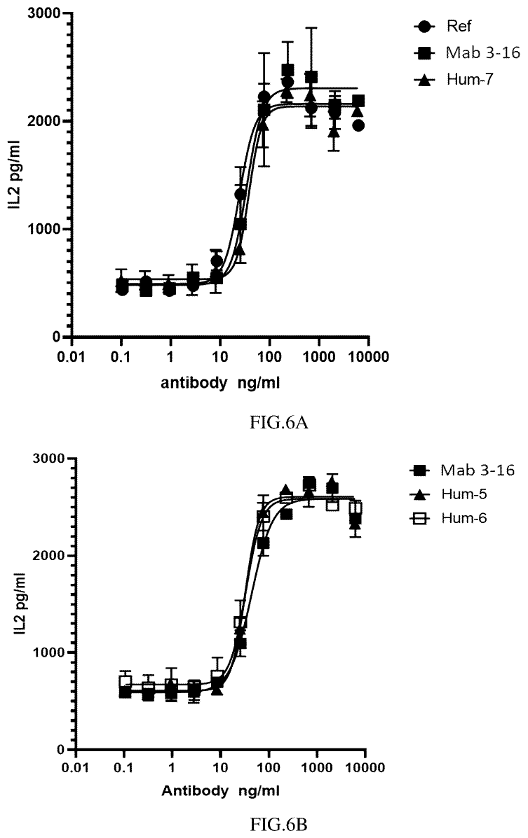

As shown in FIGS. 6A and 6B, the humanized antibodies Hum5, Hum6 and Hum7 retain their full activity in this assay when comparing with the reference antibody and parent antibody Mab 3-16 in the meaning of EC₅₀ value.

### Effect of Anti-PD-L1 Antibodies on IL-2 Secretion by Staphylococcal Enterotoxin B Stimulated PBMCs

The functionality of the humanized antibodies Hum-5, Hum-6 and Hum-7 were further evaluated by using primary cells in the assay of SEB stimulated human PBMC described as above in Example 3.

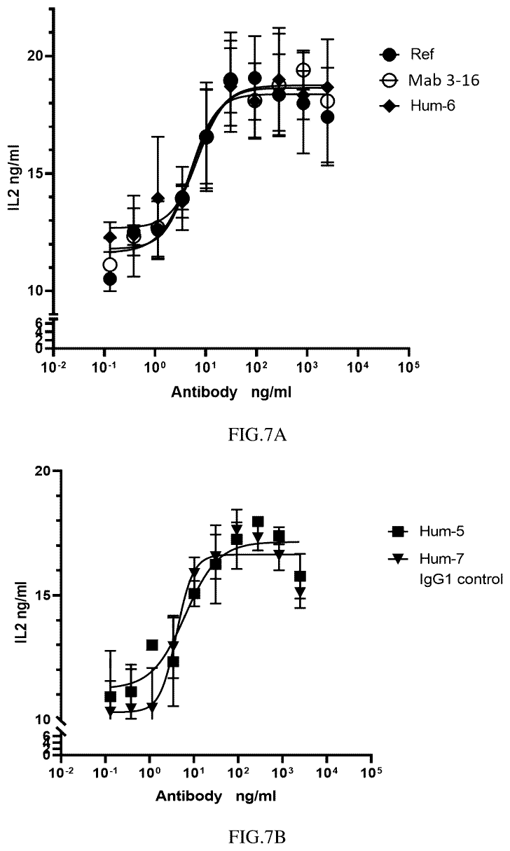

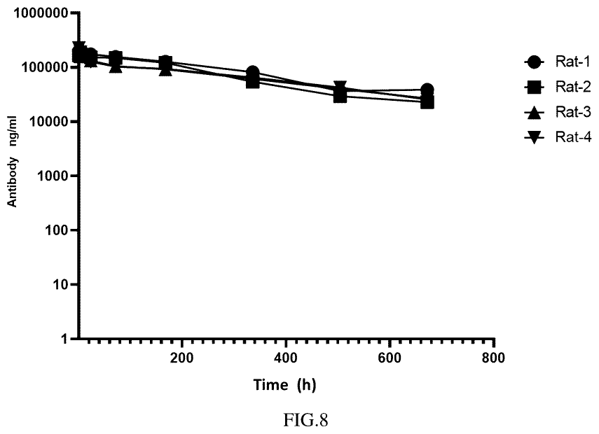

As shown in FIGS. 7A and 7B, there are no any loss of the activity of the humanized antibody Hum-5, Hum-6 and Hum-7 in promoting the IL-2 secretion of T cells in PBMC when compared with their parent antibody Mab 3-16.

### Example 6: Pharmacokinetics (PK) Study of Anti-Human PD-L1 Antibodies

PK study in rat: In a single-dose preclinical PK study, four rats were received i.v. administration of Hum-6 antibody at 10 mg/kg dosing. No obvious body weight loss or unexpected deaths were observed with the four rats during the treatment. ELISA method was chosen to determine the serum concentration of the drug by titration of the human IgG1-Fc level in the rat serums. The serum drug concentration levels prior to administration were below the lower limit of quantitation (LLOQ) of the ELISA in all individuals.

The concentration-time data of the Hum-6 antibody is shown in FIG. 8. The drug serum PK parameters are shown in Table 12.

TABLE 12

| | | T½ | AUC(0-t) | AUC(0-∞) | Cmax | Tmax | Cl | Vd |
|---|---|---|---|---|---|---|---|---|
| | k | (h) | (h*μg/ml) | (h*μg/ml) | (μg/ml) | (h) | (ml/h/kg) | (ml/kg) |
| Rat 1 | 0.0035 | 198.04 | 59776.61 | 70885.38 | 198.81 | 1 | 0.14 | 40.31 |
| Rat 2 | 0.005 | 138.63 | 50227.16 | 54790.23 | 170.96 | 8 | 0.18 | 36.50 |
| Rat 3 | 0.0045 | 154.03 | 46041.62 | 51999.95 | 216.79 | 1 | 0.19 | 42.74 |
| Rat 4 | 0.0045 | 154.03 | 46765.18 | 52467.12 | 227.94 | 1 | 0.19 | 42.35 |
| mean | 0.004375 | 161.18 | 50702.64 | 57535.67 | 203.63 | | 0.18 | 40.47 |

1. An isolated anti-PD-L1 antibody, wherein the anti-PD-L1 antibody comprises a heavy chain variable domain (V_{H}) comprising:
a heavy chain complementarity determining region (HC-CDR) 1 comprising GFTFGGFG (SEQ ID NO: 1),
an HC-CDR2 comprising ITGDSSTI (SEQ ID NO: 6), and
an HC-CDR3 comprising VRGPPGTWAY (SEQ ID NO: 11);
and a light chain variable domain (V_{L}) comprising:
a light chain complementarity determining region (LC-CDR) 1 comprising ESVEFYGTTL (SEQ TD NO: 17),
an LC-CDR2 comprising GAS (SEQ ID NO: 24), and
an LC-CDR3 comprising QQIRKVPWT (SEQ ID NO: 29).

2. The isolated anti-PD-L1 antibody of claim 1, comprising:
(i) a V_{H} comprising the amino acid sequence of SEQ ID NO: 35, or a variant thereof having at least about 80% sequence identity to the amino acid sequence of SEQ ID NO: 35; and a V_{L} comprising the amino acid sequence of SEQ ID NO: 45, or a variant thereof having at least about 80% sequence identity to the amino acid sequence of SEQ ID NO: 45;
(ii) a V_{H} comprising the amino acid sequence of SEQ ID NO: 38, or a variant thereof having at least about 80% sequence identity to the amino acid sequence of SEQ ID NO: 38; and a V_{L} comprising the amino acid sequence of SEQ ID NO: 47, or a variant thereof having at least about 80% sequence identity to the amino acid sequence of SEQ ID NO: 47;
(iii) a V_{H} comprising the amino acid sequence of SEQ ID NO: 38, or a variant thereof having at least about 80% sequence identity to the amino acid sequence of SEQ ID NO: 38; and a V_{L} comprising the amino acid sequence of SEQ ID NO: 48, or a variant thereof having at least about 80% sequence identity to the amino acid sequence of SEQ ID NO: 48; or
(iv) a V_{H} comprising the amino acid sequence of SEQ ID NO: 38, or a variant thereof having at least about 80% sequence identity to the amino acid sequence of SEQ ID NO: 38; and a V_{L} comprising the amino acid sequence of SEQ ID NO: 46, or a variant thereof having at least about 80% sequence identity to the amino acid sequence of SEQ ID NO: 46.

3-4. (canceled)

5. The isolated anti-PD-L1 antibody of claim 1, wherein the anti-PD-L1 antibody binds to the PD-L1 with a Kd from about 0.1 pM to about 1 nM.

6. The isolated anti-PD-L1 antibody according to claim 1, wherein the anti-PD-L1 antibody comprises an Fc fragment.

7. The isolated anti-PD-L1 antibody of claim 6, wherein the anti-PD-L1 antibody is a full-length IgG antibody.

8. The isolated anti-PD-L1 antibody of claim 7, wherein the anti-PD-L1 antibody is a full-length IgG1, IgG2, IgG3 or IgG4 antibody.

9. The isolated anti-PD-L1 antibody of claim 1, wherein the anti-PD-L1 antibody is chimeric, human, or humanized.

10. The isolated anti-PD-L1 antibody according to claim 1, wherein the anti-PD-L1 antibody is an antigen binding fragment selected from the group consisting of an Fab, an Fab′, an F(ab)′2, an Fab′-SH, a single-chain Fv (scFv), an Fv fragment, and a diabody.

11. An isolated nucleic acid molecule that encodes the isolated anti-PD-L1 antibody according to claim 1.

12. A vector comprising the nucleic acid molecule of claim 11.

13. An isolated host cell comprising the vector of claim 12.

14. A method of producing an isolated anti-PD-L1 antibody, comprising:
a) culturing the host cell of claim 13 under conditions effective to express the anti-PD-L1 antibody; and
b) obtaining the expressed anti-PD-L1 antibody from the host cell.

15. A pharmaceutical composition comprising the anti-PD-L1 antibody according to claim 1 and a pharmaceutically acceptable carrier.

16. A method of treating a disease or condition in an individual in need thereof, comprising administering to the individual an effective amount of the anti-PD-L1 antibody according to claim 1.

17. The method of claim 16, wherein the disease or condition is a cancer or infectious disease, optionally the disease or condition is associated with the activity and/or expression of PD-L1 or a T-cell dysfunction.

18. The method of claim 17, wherein the disease or condition is selected from melanoma, non-small cell lung cancer, hepatocellular carcinoma, gastric cancer, bladder cancer, lung cancer, renal cell carcinoma, cervical cancer, colon cancer, colorectal cancer, head and neck cancer, breast cancer, oesophageal cancer, bone cancer, prostate cancer, basal cell carcinoma, biliary tract cancer, brain and CNS cancer, choriocarcinoma, connective tissue cancer, cancer of the digestive system, endometrial cancer, eye cancer, intra-epithelial cancer, kidney cancer, larynx cancer, leukaemia, liver cancer, lung cancer, lymphoma, myeloma, neuroblastoma, oral cavity cancer, ovarian cancer, rhabdomyosarcoma, sarcoma, skin cancer, testicular cancer, thyroid cancer, uterine cancer, urinary tract cancer, and pancreatic cancer; or infectious diseases including, but not limited to Human Immunodeficiency Virus (HIV), hepatitis (A, B, or C), herpes virus (e.g. VZV, HSV-1, HAV-6, HSV-II, and CMV, Epstein Barr virus), adenovirus, influenza virus, flaviviruses, echovirus.


SEQUENCE LISTING (63 sequences)

SEQ ID NO: 1 - protein, 8 aa, synthetic construct
GFTFGGFG

SEQ ID NO: 2 - protein, 8 aa, synthetic construct
GYSFSGYF

SEQ ID NO: 3 - protein, 8 aa, synthetic construct
GYTFTDYA

SEQ ID NO: 4 - protein, 8 aa, synthetic construct
GDSITSGY

SEQ ID NO: 5 - protein, 8 aa, synthetic construct
GYTFSMYY

SEQ ID NO: 6 - protein, 8 aa, synthetic construct
ITGDSSTI

SEQ ID NO: 7 - protein, 8 aa, synthetic construct
INPSNDDT

SEQ ID NO: 8 - protein, 8 aa, synthetic construct
IIPSGDHT

SEQ ID NO: 9 - protein, 7 aa, synthetic construct
VSYSGST

SEQ ID NO: 10 - protein, 8 aa, synthetic construct
INPSNGRT

SEQ ID NO: 11 - protein, 10 aa, synthetic construct
VRGPPGTWAY

SEQ ID NO: 12 - protein, 12 aa, synthetic construct
ARFGHASWYFDV

SEQ ID NO: 13 - protein, 12 aa, synthetic construct
ARFGHASWYLDV

SEQ ID NO: 14 - protein, 13 aa, synthetic construct
ARGHGFHLYVMDY

SEQ ID NO: 15 - protein, 14 aa, synthetic construct
ANYYGDNWPFPLDY

SEQ ID NO: 16 - protein, 11 aa, synthetic construct
TREISYDEFAY

SEQ ID NO: 17 - protein, 10 aa, synthetic construct
ESVEFYGTTL

SEQ ID NO: 18 - protein, 5 aa, synthetic construct
SSVIY

SEQ ID NO: 19 - protein, 5 aa, synthetic construct
SSIIY

SEQ ID NO: 20 - protein, 6 aa, synthetic construct
QSIADY

SEQ ID NO: 21 - protein, 6 aa, synthetic construct
QSINGN

SEQ ID NO: 22 - protein, 11 aa, synthetic construct
KSLLHSNGNTY

SEQ ID NO: 23 - protein, 11 aa, synthetic construct
QSLVHSNGNTY

SEQ ID NO: 24-28 - skipped (5 entries)

SEQ ID NO: 29 - protein, 9 aa, synthetic construct
QQIRKVPWT

SEQ ID NO: 30 - protein, 9 aa, synthetic construct
QQWSTYPLT

SEQ ID NO: 31 - protein, 9 aa, synthetic construct
QNGHSFPPT

SEQ ID NO: 32 - protein, 9 aa, synthetic construct
QNGHGFPYT

SEQ ID NO: 33 - protein, 9 aa, synthetic construct
MQHLEYPFT

SEQ ID NO: 34 - protein, 9 aa, synthetic construct
SQSAHLPYT

SEQ ID NO: 35 - protein, 117 aa, synthetic construct
DVQLVESGGGLVQPGGSRKLSCAASGFTFGGFGMHWVRQAPEKGLEWVAYITGDSSTIYYADRVRGRFTISRDNPRNILFLQMTSLGSEDTAMYYCVRGPPGTWAYWGQGTLVTVSA

SEQ ID NO: 36 - protein, 117 aa, synthetic construct
EVQLVESGGGLVQPGGSLRLSCAASGFTFGGFGMNWVRQAPGKGLEWVSYITGDSSTIYYADRVRGRFTISRDNAKNILYLQMNSLGAEDTAVYYCVRGPPGTWAYWGQGTLVTVSS

SEQ ID NO: 37 - protein, 117 aa, synthetic construct
EVQLVESGGGLVQPGGSLRLSCAASGFTFGGFGMNWVRQAPGKGLEWVSYITGDSSTIYYADRVRGRFTISRDNAKNSLYLQMNSLRAEDTAVYYCVRGPPGTWAYWGQGTLVTVSS

SEQ ID NO: 38 - protein, 117 aa, synthetic construct
EVQLVESGGGLVQPGGSLRLSCAASGFTFGGFGMHWVRQAPGKGLEWVSYITGDSSTIYYADRVRGRFTISRDNAKNSLYLQMNSLRAEDTAVYYCVRGPPGTWAYWGQGTLVTVSS

SEQ ID NO: 39 - protein, 117 aa, synthetic construct
EVQLVESGGGLVQPGGSLRLSCAASGFTFGGFGMNWVRQAPGKGLEWVSYITGDSSTIYYADRVRGRFTISRDNAKNILYLQMNSLRAEDTAVYYCVRGPPGTWAYWGQGTLVTVSS

SEQ ID NO: 40 - protein, 119 aa, synthetic construct
EVQLQQSGPELVKPGASVKMSCKASGYSFSGYFLNWVKQSHGKSLEWIGRINPSNDDTVYSQKFKDRATLSLDRSSGTVHMELRSLTSEDSAVYYCARFGHASWYFDVWGAGTTVTVSS

SEQ ID NO: 41 - protein, 119 aa, synthetic construct
EVQLQQSGPELVKPGASVKMSCKASGYSFSGYFLNWVKQSHGKSLEWIGRINPSNDDTVYSQKFKDRATLSLDRSSGTVHMELRSLTSEDSAVYYCARFGHASWYLDVWGAGTTVTVSS

SEQ ID NO: 42 - protein, 120 aa, synthetic construct
QVQLQQSGAELARPGASVKMSCKASGYTFTDYAMHWIKQRPGQGLEWIGFIIPSGDHTDYNQNFKDRATLTADKSSNTAYMQLTSLTPDDSAVYFCARGHGFHLYVMDYWGQGTSVTVSS

SEQ ID NO: 43 - protein, 120 aa, synthetic construct
EVHLQESGPSLVKPSQTLSLTCSVTGDSITSGYWNWIRQFPGNKLEYMGYVSYSGSTYYNPSLKSRISITRDTSKNQYYLQLSSVTTEDTATYYCANYYGDNWPFPLDYWGQGTSVIVSS

SEQ ID NO: 44 - protein, 118 aa, synthetic construct
QVQLQQSGAELVKPGTSVKLSCKASGYTFSMYYMFWVKQRPGQGLEWIGQINPSNGRTNFNEKFKSKATLTVDKSSTTAYMQLSSLTSEDSAVYYCTREISYDEFAYWGQGTLVTVSA

SEQ ID NO: 45 - protein, 111 aa, synthetic construct
DIVLTQSPASLAVSLGQRATFSCRASESVEFYGTTLVQWYQQRPGQPPKLLIYGASNVESGVPARFTGSGSGTDFSLNIHPVEEDDIAMYFCQQIRKVPWTFGGGTKLEIR

SEQ ID NO: 46 - protein, 111 aa, synthetic construct
EIVLTQSPATLSLSPGERATLSCRASESVEFYGTTLVQWYQQKPGQAPRLLIYGASNVESGIPARFSGSGSGTDFTLTISSLEPEDFAVYFCQQIRKVPWTFGQGTKVEIK

SEQ ID NO: 47 - protein, 111 aa, synthetic construct
EIVLTQSPATLSLSPGERATLSCRASESVEFYGTTLVQWYQQKPGQAPRLLIYGASNVESGIPARFSGSGSGTDFTLTISSLEPEDFAVYYCQQIRKVPWTFGQGTKVEIK

SEQ ID NO: 48 - protein, 111 aa, synthetic construct
EIVLTQSPATLSLSPGERATLSCRASESVEFYGTTLVQWYQQKPGQPPRLLIYGASNVESGIPARFSGSGSGTDFTLTISSLEPEDFAVYYCQQIRKVPWTFGQGTKVEIK

SEQ ID NO: 49 - protein, 106 aa, synthetic construct
QIVLTQSPAIMSASPGEKVTMTCSARSSVIYMSWYQQKPGSSPRLLIYDTSHLASGVPVRFSGSGSGTFYSLIISRVEAEDAATYYCQQWSTYPLTFGAGTRLELN

SEQ ID NO: 50 - protein, 106 aa, synthetic construct
QIVLTQSPAIMSASPGEKVTMTCSARSSIIYMSWYQQKTGSSPRLLIYDTSHLASGVPVRFSGSGSGTFFSLIISRMEAEDAATYYCQQWSTYPLTFGAGTRLELK

SEQ ID NO: 51 - protein, 107 aa, synthetic construct
DIVMTQSPATLSVTPGDGVSLSCRASQSIADYLHWYQQKSHESPRLLIKYASQSISGIPSRFSGSGSGSDFTLSISSVEPEDVGVYFCQNGHSFPPTFGTGTKLELK

SEQ ID NO: 52 - protein, 107 aa, synthetic construct
DIVMTQSPATLSVTPGDRVSLSCRASQSINGNLHWFQQKSHESPRLLIKYASQSISGIPSRFSGSGSGSDFTLRINSVEPEDVGVYYCQNGHGFPYTFGGGTKLEIK

SEQ ID NO: 53 - protein, 112 aa, synthetic construct
DIVMTQAAPSVPVTPGESVSISCRSNKSLLHSNGNTYLYWFLQRPGQSPQLLIYRMSNLASGVPDRFSGSGSGTAFTLTISRVEAEDVGVYYCMQHLEYPFTFGGGTKLEIE

SEQ ID NO: 54 - protein, 112 aa, synthetic construct
DVVMTQTPLSLPVSLGDQASISCRSSQSLVHSNGNTYLHWYLQKPGQSPKLLIYRVSNRFSGVPDRFSGSGSGTDFTLKISRVEAEDLGIYFCSQSAHLPYTFGGGTKLEIK

SEQ ID NO: 55 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 107 aa, synthetic construct [sequence omitted]

SEQ ID NO: 58 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 220 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 60 - protein, 221 aa, synthetic construct
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTH

SEQ ID NO: 61 - protein, 454 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 62 - protein, 456 aa, synthetic construct
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 63 - protein, 454 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK